(19) United States
(12) Patent Application Publication
(10) Pub. No.: US 2025/0280834 A1
(43) Pub. Date: Sep. 11, 2025

(54) CONTROL OF INSECT PESTS

(71) Applicants: THE UNIVERSITY OF QUEENSLAND, St. Lucia (AU); HORTICULTURE INNOVATION AUSTRALIA LIMITED, North Sydney, New South Wales (AU); COTTON RESEARCH AND DEVELOPMENT CORPORATION, Narrabri, New South Wales (AU)

(72) Inventors: Neena MITTER, Pullenvale, Queensland (AU); Ritesh Gyanchandaji JAIN, Bald Hills, Queensland (AU); Stephen John FLETCHER, Taringa, Queensland (AU); Aris PANAYI, Laverton North, Victoria (AU); Kieran MURPHY, Laverton North, Victoria (AU)

(21) Appl. No.: 18/692,317

(22) PCT Filed: Sep. 15, 2022
(86) PCT No.: PCT/AU2022/051113

§ 371 Date: Mar. 14, 2024

(30) Foreign Application Priority Data
Sep. 15, 2021 (AU) 2021902978

(51) Int. Cl. A01N 63/60; A01P 7/04

(52) U.S. Cl. CPC A01N 63/60; A01P 7/04

(57) ABSTRACT

The present disclosure provides methods and compositions to control pests that cause damage to crop plants, for example, to control whitefly. The methods and compositions disclosed employ one or more silencing elements that decrease the level of one or more target sequences in the pest.

The text file named “208130PCT Sequence Listing_v230”, created on Nov. 15, 2024, and sized 1,727,032 bytes, which contains sequence ID listings, is herein expressly incorporated by reference.

### TECHNICAL FIELD

The disclosure relates generally to the control of pests that cause damage to crop plants, and more particularly to the control of whitefly using interfering RNA molecules.

### BACKGROUND OF INVENTION

Insect pests are major constraints to agricultural productivity, and their impacts have been reported to reduce crop yields by 10-16% globally. Several pest species are responsible for altering the natural ecosystem functioning and disrupting biodiversity. Genetically modified (GM) crops such as corn, cotton and soybean that express Bacillus thuringiensis (Bt) have been shown to be effective for controlling herbivorous pests. However, Bt technology is ineffective against sap-sucking insect pests belonging to the order Hemiptera. This group of insects comprises whiteflies, aphids, psyllids, plant bugs and leafhoppers.

Whiteflies are highly invasive and polyphagous insect pests, causing substantial economic losses to more than 600 plant species. They have gained the status of key agricultural pest due to direct damage by sap feeding, competency in virus transmission, and honeydew excretion, which promotes sooty mould on leaves and fruits and thereby reducing market value of produce. Whiteflies can transmit over 200 plant viruses and with Bemisia tabaci B and Q biotypes being the prominent agricultural pests of vegetables, fibres, and ornamental crops. The economic damage caused B. tabaci throughout the world is more than $US300 million per year.

Insecticides are the preferred way of controlling B. tabaci populations in agricultural systems. However, insecticide use has led to resistance in B. tabaci. Currently, B. tabaci has exhibited resistance to more than 60 active ingredients including neonicotinoid, organophosphate and pyrethroid insecticides.

As such, effective control measures against B. tabaci are required.

### SUMMARY OF INVENTION

Methods and compositions are provided which employ one or more silencing elements that, when ingested by or contacted with an insect plant pest, such as Bemisia tabaci; B. argentifolii Bellows & Perring; Dialeurodes citri Ashmead Trialeurodes abutiloneus, or T. vaporariorum Westwood plant pest, can decrease gene expression of one or more target sequences (e.g., mRNAs) by, for example, inhibiting translation or directly causing degradation of the target sequence(s).

In some embodiments, the methods and compositions of the invention reduce the level of one or more target sequences in the pest.

In certain embodiments, the decrease in the level of the one or more target sequences controls one or more of the pests, and thereby the methods and compositions are capable of limiting damage to a plant.

Described herein are various target polynucleotide sequences, or variants, isoforms, or fragments thereof, or complements thereof. The target sequences may be, for example, involved in growth, development, fecundity, metabolism, feeding behaviour, fitness, or insecticide tolerance of the insect plant pest. Also provided are silencing elements, which when ingested by or contacted with the pest, decrease the level of one or more of the target polynucleotide sequences.

The present inventors have surprising found that silencing specific whitefly genes results in significant whitefly mortality and that silencing elements the target RNA transcripts of these genes can be used to control whitefly. Significantly, not all genes targeted resulted in whitefly mortality.

Accordingly, the present disclosure provides a silencing element comprising at least one double-stranded RNA region, at least one strand of which comprises a polynucleotide that is complementary to a RNA transcript of:
(a) a nucleotide sequence comprising the sequence of any one of SEQ ID NOS: 1-35; or variants and fragments thereof, and complements thereof;
(b) a nucleotide sequence comprising at least 70% sequence identity to the sequence of any one of SEQ ID NOS: 1-35; or variants and fragments thereof, and complements thereof; or
(c) a nucleotide sequence comprising at least 19 consecutive nucleotides of the sequence of any one of SEQ ID NOS: 1-35; or variants and fragments thereof, and complements thereof;
   wherein the silencing element has insecticidal activity against an insect plant pest.

The present inventors have surprising found that targeting a nucleotide sequence comprising a nucleotide sequence shown in any one of SEQ ID NOS: 36-104 results in greater than 25% whitefly mortality (of adults, nymphs and/or eggs).

Accordingly, the present disclosure provides a silencing element comprising at least one double-stranded RNA region, at least one strand of which comprises a polynucleotide that is complementary to:
(a) a nucleotide sequence comprising the sequence of any one of SEQ ID NOS: 36-104; or variants, isoforms and fragments thereof, and complements thereof;
(b) a nucleotide sequence comprising at least 70% sequence identity to the sequence of any one of SEQ ID NOS: 36-104; or variants, isoforms and fragments thereof, and complements thereof; or
(c) a nucleotide sequence comprising at least 19 consecutive nucleotides of the sequence of any one of SEQ ID NOS: 36-104; or variants, isoforms and fragments thereof, and complements thereof;
   wherein the silencing element has insecticidal activity against an insect plant pest.

The present inventors have surprising found that targeting a nucleotide sequence comprising a nucleotide sequence shown in any one of SEQ ID NOS: 70-89, 91-95, or 100 results in greater than 50% whitefly mortality.

Accordingly, the present disclosure provides a silencing element comprising at least one double-stranded RNA region, at least one strand of which comprises a polynucleotide that is complementary to:
(a) a nucleotide sequence comprising the sequence of any one of SEQ ID NOS: 70-89, 91-95, 100; or variants, isoforms and fragments thereof, and complements thereof;
(b) a nucleotide sequence comprising at least 70% sequence identity to the sequence of any one of SEQ ID NOS: 70-89, 91-95, 100; or variants, isoforms and fragments thereof, and complements thereof; or
(c) a nucleotide sequence comprising at least 19 consecutive nucleotides of the sequence of any one of SEQ ID NOS: 70-89, 91-95, 100; or variants, isoforms and fragments thereof, and complements thereof;
   wherein the silencing element has insecticidal activity against an insect plant pest.

In some embodiments the silencing element comprises at least one double-stranded RNA region, at least one strand of which comprises a polynucleotide that is complementary to:
(a) a nucleotide sequence comprising the sequence any one of SEQ ID NOS: 70, 72, 74-89, 95, 100; or variants, isoforms, and fragments thereof, and complements thereof;
(b) a nucleotide sequence comprising at least 70% sequence identity to the sequence of any one of SEQ ID NOS: 70, 72, 74-89, 95, 100; or variants, isoforms and fragments thereof, and complements thereof; or
(c) a nucleotide sequence comprising at least 19 consecutive nucleotides of the sequence of any one of SEQ ID NOS: 70, 72, 74-89, 95, 100; or variants, isoforms and fragments thereof, and complements thereof.

In other embodiments, the silencing element comprises at least one double-stranded RNA region, at least one strand of which comprises a polynucleotide that is complementary to:
(a) a nucleotide sequence comprising the sequence any one of SEQ ID NOS: 70-89, 92; or variants, isoforms, and fragments thereof, and complements thereof;
(b) a nucleotide sequence comprising at least 70% sequence identity to the sequence of any one of SEQ ID NOS: 70-89, 92; or variants, isoforms and fragments thereof, and complements thereof; or
(c) a nucleotide sequence comprising at least 19 consecutive nucleotides of the sequence of any one of SEQ ID NOS: 70-89, 92; or variants, isoforms and fragments thereof, and complements thereof.

In other or further embodiments, the silencing element comprises at least one double-stranded RNA region, at least one strand of which comprises a polynucleotide that is complementary to:
(a) a nucleotide sequence comprising the sequence any one of SEQ ID NOS: 91, 93-95, 100; or variants, isoforms, and fragments thereof, and complements thereof;
(b) a nucleotide sequence comprising at least 70% sequence identity to the sequence of any one of SEQ ID NOS: 91, 93-95, 100; or variants, isoforms and fragments thereof, and complements thereof; or
(c) a nucleotide sequence comprising at least 19 consecutive nucleotides of the sequence of any one of SEQ ID NOS: 91, 93-95, 100; or variants, isoforms and fragments thereof, and complements thereof.

In other embodiments, the silencing element comprises at least one double-stranded RNA region, at least one strand of which comprises a polynucleotide that is complementary to:
(a) a nucleotide sequence comprising the sequence any one of SEQ ID NOS: 70, 72, 74, 75, 77-89; or variants, isoforms and fragments thereof, and complements thereof;
(b) a nucleotide sequence comprising at least 70% sequence identity to the sequence of any one of SEQ ID NOS: 70, 72, 74, 75, 77-89; or variants, isoforms and fragments thereof, and complements thereof; or
(c) a nucleotide sequence comprising at least 19 consecutive nucleotides of the sequence of any one of SEQ ID NOS: 70, 72, 74, 75, 77-89; or variants, isoforms and fragments thereof, and complements thereof.

In other or further embodiments, the silencing element comprises at least one double-stranded RNA region, at least one strand of which comprises a polynucleotide that is complementary to:
(a) a nucleotide sequence comprising the sequence SEQ ID NO: 95 or 100; or variants, isoforms and fragments thereof, and complements thereof;
(b) a nucleotide sequence comprising at least 70% sequence identity to the sequence of SEQ ID NOS: 95 or 100; or variants, isoforms and fragments thereof, and complements thereof; or
(c) a nucleotide sequence comprising at least 19 consecutive nucleotides of the sequence of SEQ ID NO: 95 or 100; or variants, isoforms and fragments thereof, and complements thereof.

The present inventors have surprising found that targeting a nucleotide sequence comprising a nucleotide sequence shown in any one of SEQ ID NOS: 77, 80, 82-86, 88, 91-95, or 100 results in greater than 80% whitefly mortality.

Accordingly, the present disclosure also provides a silencing element comprising at least one double-stranded RNA region, at least one strand of which comprises a polynucleotide that is complementary to:
(a) a nucleotide sequence comprising the sequence any one of SEQ ID NOS: 77, 80, 82-86, 88, 91-95, or 100; or variants, isoforms and fragments thereof, and complements thereof;
(b) a nucleotide sequence comprising at least 70% sequence identity to the sequence of any one of SEQ ID NOS: 77, 80, 82-86, 88, 91-95, or 100; or variants, isoforms and fragments thereof, and complements thereof; or
(c) a nucleotide sequence comprising at least 19 consecutive nucleotides of the sequence of any one of SEQ ID NOS: 77, 80, 82-86, 88, 91-95, or 100; or variants, isoforms and fragments thereof, and complements thereof.

In some embodiments the silencing element comprises at least one double-stranded RNA region, at least one strand of which comprises a polynucleotide that is complementary to:
(a) a nucleotide sequence comprising the sequence any one of SEQ ID NOS: 77, 80, 82-86, 88, 95, or 100; or variants, isoforms and fragments thereof, and complements thereof;

(b) a nucleotide sequence comprising at least 70% sequence identity to the sequence of any one of SEQ ID NOS: 77, 80, 82-86, 88, 95, or 100; or variants, isoforms and fragments thereof, and complements thereof; or

(c) a nucleotide sequence comprising at least 19 consecutive nucleotides of the sequence of any one of SEQ ID NOS: 77, 80, 82-86, 88, 95, or 100; or variants, isoforms and fragments thereof, and complements thereof.

In other embodiments, the silencing element comprises at least one double-stranded RNA region, at least one strand of which comprises a polynucleotide that is complementary to:
(a) a nucleotide sequence comprising the sequence any one of SEQ ID NOS: 77, 80, 82-86, 88, 92; or variants, isoforms and fragments thereof, and complements thereof;
(b) a nucleotide sequence comprising at least 70% sequence identity to the sequence of any one of SEQ ID NOS: 77, 80, 82-86, 88, 92; or variants, isoforms and fragments thereof, and complements thereof; or
(c) a nucleotide sequence comprising at least 19 consecutive nucleotides of the sequence of any one of SEQ ID NOS: 77, 80, 82-86, 88, 92; or variants, isoforms and fragments thereof, and complements thereof.

In other or further embodiments, the silencing element comprises at least one double-stranded RNA region, at least one strand of which comprises a polynucleotide that is complementary to:
(a) a nucleotide sequence comprising the sequence any one of SEQ ID NOS: 91, 93-95, 100; or variants, isoforms and fragments thereof, and complements thereof;

(b) a nucleotide sequence comprising at least 70% sequence identity to the sequence of any one of SEQ ID NOS: 91, 93-95, 100; or variants, isoforms and fragments thereof, and complements thereof; or

(c) a nucleotide sequence comprising at least 19 consecutive nucleotides of the sequence of any one of SEQ ID NOS: 91, 93-95, 100; or variants, isoforms and fragments thereof, and complements thereof.

In other embodiments, the silencing element comprises at least one double-stranded RNA region, at least one strand of which comprises a polynucleotide that is complementary to:
(a) a nucleotide sequence comprising the sequence any one of SEQ ID NOS: 77, 80, 82-86, 88; or variants, isoforms and fragments thereof, and complements thereof;
(b) a nucleotide sequence comprising at least 70% sequence identity to the sequence of any one of SEQ ID NOS: 77, 80, 82-86, 88; or variants, isoforms and fragments thereof, and complements thereof; or
(c) a nucleotide sequence comprising at least 19 consecutive nucleotides of the sequence of any one of SEQ ID NOS: 77, 80, 82-86, 88; or variants, isoforms and fragments thereof, and complements thereof.

In other or further embodiments, the silencing element comprises at least one double-stranded RNA region, at least one strand of which comprises a polynucleotide that is complementary to:
(a) a nucleotide sequence comprising the sequence of SEQ ID NO: 95 or 100; or variants, isoforms and fragments thereof, and complements thereof;
(b) a nucleotide sequence comprising at least 70% sequence identity to the sequence of SEQ ID NO: 95 or 100; or variants, isoforms and fragments thereof, and complements thereof; or
(c) a nucleotide sequence comprising at least 19 consecutive nucleotides of the sequence of SEQ ID NOS: 95 or 100; or variants, isoforms and fragments thereof, and complements thereof.

The present disclosure also provides a composition comprising the silencing element of the disclosure. The composition may comprise two or more silencing elements that target different polynucleotide sequences. The combination of two or more silencing elements (e.g., dsRNAs) may result in increased insecticidal activity, pest mortality and/or pest control.

Accordingly, the present disclosure provides a composition comprising a first and second silencing element, wherein the
(i) first silencing element comprises at least one double-stranded RNA region, at least one strand of which comprises a polynucleotide that is complementary to:
   (a) a nucleotide sequence comprising the sequence of any one of SEQ ID NOS: 36-104; or variants, isoforms and fragments thereof, and complements thereof;
   (b) a nucleotide sequence comprising at least 70% sequence identity to the sequence of any one of SEQ ID NOS: 36-104; or variants, isoforms and fragments thereof, and complements thereof; or
   (c) a nucleotide sequence comprising at least 19 consecutive nucleotides of the sequence of any one of SEQ ID NOS: 36-104; or variants, isoforms and fragments thereof, and complements thereof; and
(ii) second silencing element comprises at least one double-stranded RNA region, at least one strand of which comprises a polynucleotide that is complementary to:
   (a) a nucleotide sequence comprising the sequence of any one of SEQ ID NOS: 36-104; or variants, isoforms and fragments thereof, and complements thereof;
   (b) a nucleotide sequence comprising at least 70% sequence identity to the sequence of any one of SEQ ID NOS: 36-104; or variants, isoforms and fragments thereof, and complements thereof; or
   (c) a nucleotide sequence comprising at least 19 consecutive nucleotides of the sequence of any one of SEQ ID NOS: 36-104; or variants, isoforms and fragments thereof, and complements thereof,
      wherein the composition has insecticidal activity against an insect plant pest.

In a further embodiment, the composition comprises a third silencing element, wherein the third silencing element comprises at least one double-stranded RNA region, at least one strand of which comprises a polynucleotide that is complementary to:
(a) a nucleotide sequence comprising the sequence of any one of SEQ ID NOS: 36-104; or variants and fragments thereof, and complements thereof;
(b) a nucleotide sequence comprising at least 70% sequence identity to the sequence of any one of SEQ ID NOS: 36-104; or variants, isoforms and fragments thereof, and complements thereof; or
(c) a nucleotide sequence comprising at least 19 consecutive nucleotides of the sequence of any one of SEQ ID NOS: 36-104; or variants, isoforms and fragments thereof, and complements thereof.

In one embodiment, the composition comprising a first and second silencing element, wherein the
(i) first silencing element comprises at least one double-stranded RNA region, at least one strand of which comprises a polynucleotide that is complementary to:
   (a) a nucleotide sequence comprising the sequence of SEQ ID NO: 94 or 95; or variants, isoforms and fragments thereof, and complements thereof;
   (b) a nucleotide sequence comprising at least 70% sequence identity to the sequence of SEQ ID NO: 94 or 95; or variants, isoforms and fragments thereof, and complements thereof; or
   (c) a nucleotide sequence comprising at least 19 consecutive nucleotides of the sequence of SEQ ID NO: 94 or 95; or variants, isoforms and fragments thereof, and complements thereof; and
(ii) second silencing element comprises at least one double-stranded RNA region, at least one strand of which comprises a polynucleotide that is complementary to:
   (a) a nucleotide sequence comprising the sequence of SEQ ID NO: 91; or variants and fragments thereof, and complements thereof;
   (b) a nucleotide sequence comprising at least 70% sequence identity to the sequence of SEQ ID NO: 91; or variants and fragments thereof, and complements thereof; or
   (c) a nucleotide sequence comprising at least 19 consecutive nucleotides of the sequence of SEQ ID NO: 91; or variants and fragments thereof, and complements thereof; and
      wherein the composition has insecticidal activity against an insect plant pest.

The present inventors have surprisingly found that the insecticidal activity of a silencing element can be improved by combination with a silencing element targeting the Deoxyribonuclease I (Nuclease-II).

Accordingly, the present disclosure provides a composition comprising a first and second silencing element, wherein the
(i) first silencing element comprises at least one double-stranded RNA region, at least one strand of which comprises a polynucleotide that is complementary to:
   (a) a nucleotide sequence comprising the sequence of SEQ ID NO: 101; or variants, isoforms and fragments thereof, and complements thereof;
   (b) a nucleotide sequence comprising at least 70% sequence identity to the sequence of SEQ ID NO: 101; or variants, isoforms and fragments thereof, and complements thereof; or
   (c) a nucleotide sequence comprising at least 19 consecutive nucleotides of the sequence of SEQ ID NO: 101; or variants, isoforms and fragments thereof, and complements thereof; and
(iii) second silencing element comprises at least one double-stranded RNA region, at least one strand of which comprises a polynucleotide that is complementary to:
   (a) a nucleotide sequence comprising the sequence of any one of SEQ ID NO: 36-104; or variants, isoforms and fragments thereof, and complements thereof;
   (b) a nucleotide sequence comprising at least 70% sequence identity to the sequence of SEQ ID NO: 36-104; or variants, isoforms and fragments thereof, and complements thereof; or
   (c) a nucleotide sequence comprising at least 19 consecutive nucleotides of the sequence of SEQ ID NO: 36-104; or variants, isoforms and fragments thereof, and complements thereof; and
      wherein the composition has insecticidal activity against an insect plant pest.

In one embodiment, the second silencing element comprises at least one double-stranded RNA region, at least one strand of which comprises a polynucleotide that is complementary to:
(a) a nucleotide sequence comprising the sequence of SEQ ID NO: 98 or 99; or variants, isoforms and fragments thereof, and complements thereof;
(b) a nucleotide sequence comprising at least 70% sequence identity to the sequence of SEQ ID NO: 98 or 99; or variants, isoforms and fragments thereof, and complements thereof; or
(c) a nucleotide sequence comprising at least 19 consecutive nucleotides of the sequence of SEQ ID NO: 98 or 99; or variants, isoforms and fragments thereof, and complements thereof.

In some embodiments the two or more silencing elements are provided as a single polynucleotide (e.g., dsRNA). In other embodiments, two or more polynucleotides (e.g., dsRNAs) are mixed together.

The present disclosure also provides a silencing element comprising at least one double-stranded RNA region, at least one strand of which comprises:
(a) a nucleotide sequence comprising the sequence of any one of SEQ ID NOS: 139-173; or variants and fragments thereof, and complements thereof;
(b) a nucleotide sequence comprising at least 70% sequence identity to the sequence of any one of nucleotides SEQ ID NOS: 139-173; or variants and fragments thereof, and complements thereof; or
(c) a nucleotide sequence comprising at least 19 consecutive nucleotides of the sequence of any one of SEQ ID NOS: 139-173; or variants and fragments thereof, and complements thereof;
   wherein the silencing element has insecticidal activity against an insect plant pest.

The present disclosure also provides a silencing element comprising at least one double-stranded RNA region, at least one strand of which comprises:
(a) a nucleotide sequence comprising the sequence any one of SEQ ID NOS: 139-158, 160-164, 169; or variants and fragments thereof, and complements thereof;
(b) a nucleotide sequence comprising at least 70% sequence identity to the sequence of any one of SEQ ID NOS: 139-158, 160-164, 169; or variants and fragments thereof, and complements thereof; or
(c) a nucleotide sequence comprising at least 19 consecutive nucleotides of the sequence of any one of SEQ ID NOS: 139-158, 160-164, 169; or variants and fragments thereof, and complements thereof.

The present disclosure also provides a silencing element comprising at least one double-stranded RNA region, at least one strand of which comprises:
(a) a nucleotide sequence comprising the sequence any one of SEQ ID NOS: 146, 149, 151-155, 157, 160-164, 169; or variants and fragments thereof, and complements thereof;
(b) a nucleotide sequence comprising at least 70% sequence identity to the sequence of any one of SEQ ID NOS: 146, 149, 151-155, 157, 160-164, 169; or variants and fragments thereof, and complements thereof; or
(c) a nucleotide sequence comprising at least 19 consecutive nucleotides of the sequence of any one of SEQ ID NOS: 146, 149, 151-155, 157, 160-164, 169; or variants and fragments thereof, and complements thereof.

The present disclosure also provides a silencing element comprising at least one double-stranded RNA region, at least one strand of which comprises:
(a) a nucleotide sequence comprising the sequence any one of SEQ ID NOS: 139-158, 161; or variants and fragments thereof, and complements thereof;
(b) a nucleotide sequence comprising at least 70% sequence identity to the sequence of any one of SEQ ID NOS: 139-158, 161; or variants and fragments thereof, and complements thereof; or
(c) a nucleotide sequence comprising at least 19 consecutive nucleotides of the sequence of any one of SEQ ID NOS: 139-158, 161; or variants and fragments thereof, and complements thereof.

The present disclosure also provides a silencing element comprising at least one double-stranded RNA region, at least one strand of which comprises:
(a) a nucleotide sequence comprising the sequence any one of SEQ ID NOS: 146, 149, 151-155, 157, 161; or variants and fragments thereof, and complements thereof;
(b) a nucleotide sequence comprising at least 70% sequence identity to the sequence of any one of SEQ ID NOS: 146, 149, 151-155, 157, 161; or variants and fragments thereof, and complements thereof; or
(c) a nucleotide sequence comprising at least 19 consecutive nucleotides of the sequence of any one of SEQ ID NOS: 146, 149, 151-155, 157, 161; or variants and fragments thereof, and complements thereof.

The present disclosure also provides a silencing element comprising at least one double-stranded RNA region, at least one strand of which comprises:
(a) a nucleotide sequence comprising the sequence any one of SEQ ID NOS: 160, 162-164, 169; or variants and fragments thereof, and complements thereof;
(b) a nucleotide sequence comprising at least 70% sequence identity to the sequence of any one of SEQ ID NOS: 160, 162-164, 169; or variants and fragments thereof, and complements thereof; or
(c) a nucleotide sequence comprising at least 19 consecutive nucleotides of the sequence of any one of SEQ ID NOS: 160, 162-164, 169; or variants and fragments thereof, and complements thereof.

Accordingly, the present disclosure provides a composition comprising a first and second silencing element, wherein the
(i) first silencing element comprises at least one double-stranded RNA region, at least one strand of which comprises:
   (a) a nucleotide sequence comprising the sequence of any one of SEQ ID NOS: 139-173; or variants and fragments thereof, and complements thereof;
   (b) a nucleotide sequence comprising at least 70% sequence identity to the sequence of any one of nucleotides SEQ ID NOS: 139-173; or variants, and fragments thereof, and complements thereof; or
   (c) a nucleotide sequence comprising at least 19 consecutive nucleotides of the sequence of any one of SEQ ID NOS: 139-173; or variants and fragments thereof, and complements thereof; and
(ii) second silencing element comprises at least one double-stranded RNA region, at least one strand of which comprises:
   (a) a nucleotide sequence comprising the sequence of any one of SEQ ID NOS: 139-173; or variants and fragments thereof, and complements thereof;
   (b) a nucleotide sequence comprising at least 70% sequence identity to the sequence of any one of SEQ ID NOS: 139-173; or variants, and fragments thereof, and complements thereof; or
   (c) a nucleotide sequence comprising at least 19 consecutive nucleotides of the sequence of any one of SEQ ID NOS: 139-173; or variants and fragments thereof, and complements thereof,
      wherein the composition has insecticidal activity against an insect plant pest.

In one embodiment, the composition comprises a third silencing element, wherein the third silencing element comprises at least one double-stranded RNA region, at least one strand of which comprises:
(a) a nucleotide sequence comprising the sequence of any one of SEQ ID NOS: 139-173; or variants and fragments thereof, and complements thereof;
(b) a nucleotide sequence comprising at least 70% sequence identity to the sequence of any one of SEQ ID NOS: 139-173; or variants, and fragments thereof, and complements thereof; or
(c) a nucleotide sequence comprising at least 19 consecutive nucleotides of the sequence of any one of SEQ ID NOS: 139-173; or variants and fragments thereof, and complements thereof.

Accordingly, the present disclosure provides a composition comprising a first and second silencing element, wherein the
(i) first silencing element comprises at least one double-stranded RNA region, at least one strand of which comprises:
   (a) a nucleotide sequence comprising the sequence of SEQ ID NO: 163 or 164; or variants and fragments thereof, and complements thereof;
   (b) a nucleotide sequence comprising at least 70% sequence identity to the sequence of SEQ ID NO: 163 or 164; or variants, and fragments thereof, and complements thereof; or
   (c) a nucleotide sequence comprising at least 19 consecutive nucleotides of the sequence of SEQ ID NO: 163 or 164; or variants and fragments thereof, and complements thereof; and
(ii) second silencing element comprises at least one double-stranded RNA region, at least one strand of which comprises:
   (a) a nucleotide sequence comprising the sequence of SEQ ID NO: 160; or variants and fragments thereof, and complements thereof;
   (b) a nucleotide sequence comprising at least 70% sequence identity to the sequence of SEQ ID NO: 160; or variants, and fragments thereof, and complements thereof; or
   (c) a nucleotide sequence comprising at least 19 consecutive nucleotides of the sequence of SEQ ID NO: 160; or variants and fragments thereof, and complements thereof,
      wherein the composition has insecticidal activity against an insect plant pest.

The present disclosure provides a composition comprising a first and second silencing element, wherein the
(i) first silencing element comprises at least one double-stranded RNA region, at least one strand of which comprises:
   (a) a nucleotide sequence comprising the sequence of SEQ ID NO: 170; or variants and fragments thereof, and complements thereof;
   (b) a nucleotide sequence comprising at least 70% sequence identity to the sequence of SEQ ID NO: 170; or variants, and fragments thereof, and complements thereof; or
   (c) a nucleotide sequence comprising at least 19 consecutive nucleotides of the sequence of SEQ ID NO: 170; or variants and fragments thereof, and complements thereof; and
(ii) second silencing element comprises at least one double-stranded RNA region, at least one strand of which comprises:
   (a) a nucleotide sequence comprising the sequence of any one of SEQ ID NOS: 139-173; or variants and fragments thereof, and complements thereof;
   (b) a nucleotide sequence comprising at least 70% sequence identity to the sequence of any one of SEQ ID NOS: 139-173; or variants, and fragments thereof, and complements thereof; or
   (c) a nucleotide sequence comprising at least 19 consecutive nucleotides of the sequence of any one of SEQ ID NOS: 139-173; or variants and fragments thereof, and complements thereof,
      wherein the composition has insecticidal activity against an insect plant pest.

In one embodiment, the second silencing element comprises at least one double-stranded RNA region, at least one strand of which comprises a polynucleotide that is complementary to:
(a) a nucleotide sequence comprising the sequence of SEQ ID NO: 167 or 168; or variants, isoforms and fragments thereof, and complements thereof;
(b) a nucleotide sequence comprising at least 70% sequence identity to the sequence of SEQ ID NO: 167 or 168; or variants, isoforms and fragments thereof, and complements thereof; or
(c) a nucleotide sequence comprising at least 19 consecutive nucleotides of the sequence of SEQ ID NO: 167 or 168; or variants, isoforms and fragments thereof, and complements thereof.

In some embodiments the two or more silencing elements are provided as a single polynucleotide (e.g., dsRNA). In other embodiments, two or more polynucleotides (e.g., dsRNAs) are mixed together.

Further provided are constructs (for example, expression constructs) encoding silencing elements

Two or more polynucleotides encoding silencing elements (e.g., dsRNAs) may be stacked into one construct.

In some embodiments, the construct is a DNA construct comprising one or more polynucleotides encoding one or more silencing elements of the disclosure.

In other embodiments, the construct is an expression construct comprising the DNA construct of the disclosure, wherein the one or more polynucleotides may be operably linked to one or more heterologous promoters.

In some embodiments, the polynucleotide is flanked by a first operably linked convergent promoter at one terminus of the polynucleotide and a second operably linked convergent promoter at the opposing terminus of the polynucleotide, wherein the first and the second convergent promoters are capable of driving expression of the silencing element.

Also provided are host cells, for example, bacterial cells, comprising one or more constructs encoding silencing elements of the disclosure. In some embodiments, the host cell is an inactivated bacterial cell.

In some embodiments, the host cell comprises an expression construct which comprises a transcriptional promoter operably linked to a DNA construct of the disclosure. The transcriptional promoter may be inducible by exposure of the host cell to an exogenous molecule, for example, IPTG.

Also provided are compositions comprising one or more sequence elements of the disclosure, one or more constructs of the disclosure, or one or more host cells of the disclosure. The composition may be in the form of a solid, suspension or colloid.

In some embodiments, the compositions comprise an agriculturally acceptable carrier. The carrier may facilitate application of the composition to plants to be protected from pests.

The compositions may also comprise an herbicide, an insecticide, a fungicide, a nematocide, and/or a bactericide, or combinations thereof.

In some embodiments, the composition is sprayable onto the leaves of the plant.

In some embodiments, the one or more silencing elements are adsorbed onto a carrier, for example, a synthetic carrier such as LDH particles. The LDH may be of the hydrotalcite group.

In some embodiments, the one or more silencing elements are loaded onto the LDH at a ratio of from 1:1 to 1:5 silencing element:LDH.

In some embodiments, 60% to 90% of the one or more silencing elements in the composition are adsorbed onto the LDH.

In some embodiments, the compositions comprise a penetrant which enhances the penetration of active compounds (e.g., silencing element or silencing element-LDH) into the plant, plant pest or both plant and plant pest.

Typically, the penetrant is adapted to dissolve or penetrate the wax layer on the leaf surface of plants. Examples of suitable penetrant may, for example, be selected from the group consisting of mineral oils, vegetable oils, esterified vegetable oils, fatty acid esters, polyalkoxylate surfactants, sugar-based surfactants and mixtures thereof.

The composition may comprise further components such an inert carrier, a preservative, a humectant, thickener, antifreeze, an encapsulating agent, a binder, an emulsifier, a dye, a UV protectant, a buffer, a flow agent, micronutrient donors, or other preparations that influence plant growth.

Plants, plant parts, plant cells, and other host cells (e.g., bacterial cells) or organisms comprising constructs encoding the silencing elements or an active variant or fragment thereof are also provided.

The disclosure provides a plant cell having stably incorporated into its genome a heterologous polynucleotide encoding a silencing element, wherein the polynucleotide comprises:
(a) a nucleotide sequence comprising the sequence of any one of SEQ ID NOS: 139-173; or variants and fragments thereof, and complements thereof;
(b) a nucleotide sequence comprising at least 70% sequence identity to the sequence of any one of SEQ ID NOS: 139-173; or variants and fragments thereof, and complements thereof; or
(c) a nucleotide sequence comprising at least 19 consecutive nucleotides of the sequence of any one of SEQ ID NOS: 139-173; or variants and fragments thereof, and complements thereof;
   wherein the silencing element has insecticidal activity against a plant pest.

In some embodiments, the plant cell comprises one or more constructs of the disclosure, encoding, dsRNAs for example.

In some embodiments, the plant cell is from a dicot such as, but not limited to, dicots. Examples of plant species of interest include, but are not limited to Row crops, Soybean, Cotton, Peanuts, Beans, Carrots, Tomato, Broccoli, Lettuce, Cucurbit crops such as Cucumber, Watermelon, Squash, Capsicum, Cabbage, Sweet potato, Eggplant, Ornamentals, Citrus, Chillies.

Also provided is a plant or plant part comprising the plant cell of the disclosure.

Also provided is a transgenic seed from the plant of the disclosure.

Also provided are formulations of sprayable silencing agents for topical applications to pest insects or substrates where pest insects may be found.

In another embodiment, methods for controlling an insect plant pest, such as, Bemisia tabaci; B. argentifolii Bellows & Perring; Dialeurodes citri Ashmead Trialeurodes abutiloneus, or T. vaporariorum Westwood plant pest, are provided. The methods comprise feeding to an insect plant pest a composition comprising one or more silencing elements, wherein the one or more silencing elements, when ingested by the pest, reduce the level of one or more target sequences in the pest and thereby control the pest. Alternatively, or in addition, the methods may comprise contacting the insect plant pest with a composition comprising one or more silencing elements, wherein the one or more silencing elements, when absorbed by the pest (e.g., through the insect cuticle), reduce the level of one or more target sequences in the pest and thereby control the pest.

Further provided are methods to protect a plant from an insect plant pest. Such methods comprise introducing into the plant or plant part a disclosed silencing element. When the plant expressing the silencing element is ingested by the pest, the level of the target sequence is decreased, and the pest is controlled.

In one embodiment, the disclosure provides a method for controlling an insect plant pest comprising feeding to the insect plant pest a composition comprising a silencing element, wherein the silencing element controls the plant pest, wherein the silencing element comprises a sequence complementary to:
(a) a nucleotide sequence comprising the sequence of any one of SEQ ID NOS: 36-104; or variants, isoforms and fragments thereof, and complements thereof;
(b) a nucleotide sequence comprising at least 70% sequence identity to the sequence of any one of nucleotides SEQ ID NOS: 36-104; or variants, isoforms and fragments thereof, and complements thereof; or
(c) a nucleotide sequence comprising at least 19 consecutive nucleotides of the sequence of any one of SEQ ID NOS: 36-104; or variants, isoforms and fragments thereof, and complements thereof;
   wherein the silencing element has insecticidal activity against an insect plant pest.

In another embodiment, the disclosure provides a method for controlling an insect plant pest comprising feeding to the insect plant pest a composition comprising a silencing element, wherein the silencing element controls the plant pest, wherein the silencing element comprises:
(a) a nucleotide sequence comprising the sequence of any one of SEQ ID NOS: 139-173; or variants and fragments thereof, and complements thereof;
(b) a nucleotide sequence comprising at least 70% sequence identity to the sequence of any one of SEQ ID NOS: 139-173; or variants and fragments thereof, and complements thereof; or
(c) a nucleotide sequence comprising at least 19 consecutive nucleotides of the sequence of any one of SEQ ID NOS: 139-173; or variants and fragments thereof, and complements thereof;
   wherein the silencing element has insecticidal activity against a plant pest.

In some embodiments, the composition comprises a plant or plant part treated with the silencing element adsorbed onto LDH particles.

In other embodiments, the composition comprises a plant or plant part having stably incorporated into its genome a polynucleotide encoding the silencing element. In some embodiments, the plant is a dicot such as, but not limited to, dicots. Examples of plant species of interest include, but are not limited to Row crops, Soybean, Cotton, Peanuts, Beans, Carrots, Tomato, Broccoli, Lettuce, Cucurbit crops such as Cucumber, Watermelon, Squash, Capsicum, Cabbage, Sweet potato, Eggplant, Ornamentals, Citrus, Chillies.

Also provided are kits comprising one or more silencing element of the disclosure, or one or more constructs of the disclosure, and instructions for using the silencing elements, or constructs as insecticidal agents against a plant pest.

In some embodiments, the instructions provide for sequential application of two or more silencing elements to reduce the incidence of the insect pest organism developing resistance to the one or more silencing elements.

In other embodiments, the instructions provide for concurrent application of two or more silencing elements to reduce the incidence of the insect pest organism developing resistance to the one or more silencing elements.

The present disclosure is not to be limited in scope by the specific examples described herein, which are intended for the purpose of exemplification only. Functionally-equivalent products, compositions and methods are clearly within the scope of the present disclosure.

Any example/embodiment of the present disclosure herein shall be taken to apply mutatis mutandis to any other example/embodiment of the disclosure unless specifically stated otherwise.

### BRIEF DESCRIPTION OF DRAWINGS

FIG. 1. Diet-mediated delivery of naked dsRNA and LDH-dsRNA (BioClay) causes whitefly mortality. a, Percentage mortality of adult whiteflies after feeding on artificial diet (AD) containing dsRNAs at a concentration of 200 ng/μl for 6 days. Sucrose and Green fluorescent protein (GFP) dsRNA were used as negative controls. AD assays were performed twice for each target dsRNA. Of the selected dsRNAs, fourteen dsRNA caused more than 80% mortality. b, Representative images of adult whiteflies after feeding on sucrose and GFP dsRNA and dead individuals after feeding on target dsRNA. c, Relative mRNA expression of selected B. tabaci genes after 4 days feeding on diet containing target dsRNAs. Relative mRNA levels of Aquaporin 1 (Aqp1), vacuolar ATPase A (v-ATPase A), Coatomer beta subunit (COPB1), Zinc finger protein (ZFP), Charged Multivesicular Body Protein 4C (CHM4C), Trehalase (TreH), and Acetylcholinesterase 1 (AchE1) were normalized using a rpl-6 as a reference gene. Of the tested target genes, Aqp1 showed the most significant gene knockdown on day four. d, The mortality of stingless bees feeding on syntaxin dsRNA via cotton pad soaking bioassays. e, Loading of dsRNA on LDH pre and post 6 days feeding. Complete loading was achieved at a dsRNA-LDH mass ratio of 1:4 and verified by agarose gel electrophoresis. The gel image indicates the differences in the migration of free dsRNA and LDH-dsRNA complexes. f, Adult whiteflies were fed on artificial diet containing naked AChE1, Aqp1, v-ATPase A dsRNAs or LDH bounded respective dsRNAs and mortality were recorded for 6 days. Of the selected target genes, Aqp1 caused the most significant whitefly mortality on day 6. Each AD assay was repeated two times. M=1 kb+ ladder. g, Relative expression of selected target genes in adult B. tabaci fed on diet mixed with GFP dsRNA or naked AChE1 (a), Aqp1 (b), v-ATPase A (c) dsRNA or corresponding LDH loaded dsRNA for four days. For qRT PCR, relative expression was normalized to an endogenous control rpl-6. Of the three selected target genes, Aqp1 bounded LDH showed the most significant gene silencing in B. tabaci as compared to GFP dsRNA. The asterisks above the bar represent the significant difference between controls and treatments (One-way ANOVA, Tukey's test was used for mortality studies and Student's t-test followed by Welch's correction performed for gene knockdown analysis *P<0.05, **P<0.001, ****P<0.0001).

FIG. 2. Screening of potential targets genes in whitefly. Percentage mortality of adult whiteflies after feeding on artificial diet (AD) containing dsRNAs at a concentration of 200 ng/μl for 6 days. Sucrose and Green fluorescent protein (GFP) dsRNA were used as negative controls. AD assays were performed twice for each target dsRNA. Of the selected dsRNAs, twelve dsRNA caused less than 80% mortality. AD assays were performed twice for each target dsRNA. Data represent the mean±SEM of three biological replicates. The asterisks above the bar represent the significant difference between controls and treatments (One-way ANOVA, Tukey's test *P<0.05, **P<0.001, ****P<0.0001).

FIG. 3. Combinations of dsRNA improve RNAi-mediated control of whitefly. a-c, Percentage mortality of B. tabaci adults were fed on diet containing 75 ng/μl of single dsRNA or 150 ng/μl of mixture of two dsRNAs (75 ng/μl each dsRNA) targeting AChE1, v-ATPase A, and ZFP and mortality was recorded for 6 days. Sucrose and GFP dsRNA were used as negative controls. On day 6, combinations of dsRNAs AChE1 and v-ATPase A (a), AChE1 and ZFP (b), and v-ATPase A and ZFP (c) caused a significant whitefly mortality as compared to the respective single dsRNA. Each bioassay was repeated two times. d-i, Relative expression of target genes in adult B. tabaci post-feeding on diet containing single dsRNA or mixture of two dsRNAs targeting AChE1, v-ATPase A, and ZFP. Four days after feeding, RNA was extracted and used in qRT-PCR to determine relative mRNA levels. On day 4, the selected single dsRNA or mixture of dsRNAs AChE1 and v-ATPase A (d and e), AChE1 and ZFP (f and g), and v-ATPase A and ZFP (h and i) caused a significant gene silencing of the target gene in B. tabaci as compared to control GFP dsRNA. j, B. tabaci adults were fed on diet containing 100 ng/μl mixture of v-ATPase A and ZFP dsRNA (50 ng/μl each dsRNA) or stacked v-ATPase A and ZFP and mortality was recorded for 6 days. Sucrose and GFP dsRNA were used as negative controls. Stacking of two dsRNAs caused significantly higher whitefly mortality than a mixture of respective dsRNAs. k-l. Relative expression of target genes in adult B. tabaci post-feeding on diet containing mixture of two dsRNAs or stacked dsRNA targeting v-ATPase A and ZFP. Four day after feeding, RNA was extracted and used in qRT-PCR to determine relative mRNA levels. On day 4, stacked dsRNAs caused a significant target gene knockdown as compared to mixture of dsRNAs and control GFP dsRNA, whereas mixture of dsRNAs did not cause significant difference than control in B. tabaci.

FIG. 4. Loading profile dsRNA to LDH. Loading of dsRNA at the dsRNA-LDH (BioClay) mass ratios of 1:1, 1:2, 1:3, and 1:4, corresponding to lanes 2, 3, 4, and 5, respectively. M=1 kb+ ladder, dsRNA only (lane 1). The dsRNA loading includes in-vitro synthesized Cystein-Glycine dsRNA. As dsRNA completely loads onto the LDH, their mobility through the well is restricted, causing dsRNA to remain in the wells at the top of gel. Complete loading for dsRNA was achieved at a dsRNA-LDH mass ratio of 1:4 (lane 5).

FIG. 5. Topical application of BioClay (dsRNA-LDH) control developmental stages of whitefly. a, Percentage B. tabaci egg and nymph mortality caused by foliar spray of dsRNA-LDH (BioClay). Cotton plants at the two-leaf stage were sprayed with water, LDH, CMV2b dsRNA, naked target dsRNA and target dsRNA-LDH on day 1 and day 10 post eggs infestation (n=6 leaves per treatment group). Mortality of eggs and nymphs was counted on day 17 post spray. b-c, Percentage eggs and nymph mortality caused by mixing of sucrase-dsRNases2 dsRNA-LDH (b) and DUOX-dsRNase2-LDH (c) 17 days post spray. d, Images showing mortality of B. tabaci eggs and nymphs 17 days post LDH-dsRNA (BioClay) spray. Eggs and nymphs on water sprayed plants (a-d). Eggs hatching into nymphs (a); newly emerged nymph (b); and advanced nymphal stages (c-d). Mortality of eggs and nymphs on cotton plants sprayed with dsRNA-LDH (e-h). Unhatched eggs (e); dead newly emerged and advanced nymphs (f-h). e,f,g Percentage B. tabaci adult mortality caused by foliar spray of naked dsRNA and LDH-dsRNA (BioClay). Cotton plants at the two-leaf stage were sprayed with water, LDH, CMV2b dsRNA, naked target dsRNA and target dsRNA-LDH. Adult whiteflies (200 per treatment group) were released into clip cages post 1 day spray (n=6-8 leaves per treatment group). Mortality was recorded for 8 days. Bioassays were performed twice for each target dsRNA. (h) The reduction in target mRNA expression levels in adult whiteflies were analyzed 6 days after feeding on treated plants by RT-qPCR. The transcript expression levels were normalized using the reference gene rpl6 relative to the CMV2b control. The asterisks above the bar represent the significant difference between controls and treatments (One-way ANOVA, Tukey's test *P<0.05, ***P<0.001, ****P<0.0001). Data represent mean+s.e.m.

FIG. 6. RNAi-mediated silencing of Sucrase reduces honeydew secretion of adult whiteflies on cotton plants. Whiteflies fed on cotton plants sprayed with sucrase dsRNA and Sucrase-BioClay showed significant reduction in honeydew secretion as compared to the adults fed on control plants.

FIG. 7. Addition of penetrant can improve uptake of dsRNA in plants. Confocal imaging of adaxial surface of cotton leaves 24 h post treatment with and without penetrant after washing. Images are shown for Cy3 only; CMV2b-dsRNA-Cy3; CMV2b-dsRNA-Cy3-banjo penetrant; CMV2b-dsRNA-Cy3-pulse penetrant; and CMV2b-dsRNA-Cy3-supercharge elite penetrant. Bright-field (BF) images (column 1), Cy3 fluorescence images (column 2) and merged images (column 3) are shown. CMV2b-dsRNA with and without penetrant pipetted on adaxial surface of cotton leaves and sampled after 24 h incubation. The adaxial surface of leaves were rinsed with water by vigorous pipetting before being viewed under confocal microscope. showing the CMV2b-dsRNA with penetrant is transported deeper in the leaf as compared to the CMV2b-dsRNA without penetrant. Scale bar=100 μm. All leaves treated with either of the penetrant showed abundant uptake and deeper penetration of Cy3 into the spongy mesophyll and plants vascular bundle.

FIG. 8. Confocal image followed with Z-stack analysis show increased uptake of Cy3 fluorophore attached to CMV-2b dsRNA by the incorporation of penetrants. Confocal microscope z-stack projections showing the CMV2b-dsRNA with penetrant is transported abundantly into the plants vascular bundle as compared to the CMV2b-dsRNA without penetrant. Scale bar=100 μm.

FIG. 9. Translaminar movement of topically applied dsRNA in plants. Confocal imaging of abaxial surface of cotton leaves 24 h post treatment with and without penetrant. Images are shown for Cy3 only; CMV2b-dsRNA-Cy3; CMV2b-dsRNA-Cy3-banjo penetrant; CMV2b-dsRNA-Cy3-pulse penetrant; and CMV2b-dsRNA-Cy3-supercharge elite penetrant. Bright-field (BF) images (column 1), Cy3 fluorescence images (column 2) and merged images (column 3) are shown. CMV2b-dsRNA with and without penetrant pipetted on adaxial surface of cotton leaves and abaxial surface were viewed under confocal microscope. Confocal microscopy visualization shows that CMV2b-dsRNA without penetrant could not be detected from abaxial surface of leaf, whereas CMV2b-dsRNA with penetrant can still be detected. This confirms that penetrant can assist in the translaminar movement of dsRNA in the leaves. Scale bar=100 μm.

FIG. 10. Uptake of BioClay released dsRNA in plants vascular bundle. Confocal imaging of cotton leaves 72 h post treatment with naked dsRNA and BioClay. Images are shown for Cy3 only, CMV2b-dsRNA-Cy3 with and without penetrants (Banjo, Pulse, Supercharge Elite). Cy3-labelled naked dsRNA and LDH-dsRNA (BioClay) were applied on the cotton leaves and viewed under confocal microscope. The naked dsRNA and BioClay treated leaves showed uptake of dsRNA into the plants vascular bundle. Scale bar=100 μm.

FIG. 11. Uptake of clay released dsRNA into plants. Confocal imaging of cotton leaves 72 h post treatment with naked dsRNA and BioClay. Images are shown for Cy3 only, CMV2b-dsRNA-Cy3 with and without penetrants (Banjo, Pulse, Supercharge Elite). Cy3-labelled naked dsRNA and LDH-dsRNA (BioClay) were applied on the cotton leaves and viewed under confocal microscope. The naked dsRNA and BioClay treated leaves showed uptake of dsRNA into the spongy mesophyll. Scale bar=100 μm.

FIG. 12. Diet-mediated uptake of dsRNA in whitefly B. tabaci. Cy3-labelled GFP dsRNA was mixed with artificial diet (sucrose 30%) at a final concentration of 100 ng/μl and fed to adult whitefly for 24 h. Confocal microscopy of whole insect illustrates localization of Cy3-labelled GFP dsRNA in the abdomen of the whitefly. Control whiteflies fed on diet without Cy3-dsRNA did not show fluorescent signal. Bright-field (BF) image (column 1), Cy3 florescence image (column 2) and merged image of the two (column 3) are shown.

FIG. 13. Whitefly uptake dsRNA through artificial diet assay. Detection of fluorescently labelled GFP-dsRNA in adult whiteflies exposed to GFP dsRNA for 24 hours. Real time (qPCR results) followed with gel electrophoresis showing amplification of GFP-dsRNA in whiteflies fed on artificial diet enriched with GFP-dsRNA but not in sucrose only diets. Gel electrophoresis of the resultant qPCR products confirms the presence of dsRNA in the correct size range (approx. 300 bp).

FIG. 14. Leaf-mediated uptake of dsRNA in whitefly B. tabaci. Confocal microscopy of the whole insect indicates localization of Cy3 signal in the abdomen of the whitefly 48 hrs after feeding on the detached cotton leaf dipped in a fluorophore-labelled CMV 2b dsRNA solution. Control whiteflies fed on detached leaf dipped in a water as a negative control did not show fluorescent signal. Bright-field (BF) image (column 1), Cy3 florescence image (column 2) and merged image of the two (column 3) are shown.

FIG. 15. Whitefly uptake dsRNA through petiole dip assay. Uptake of Cy3-labeled CMV2b-dsRNA by adult whitefly B. tabaci. Northern blot analysis of the total RNA extracted from adult whiteflies fed on CMV-2b dsRNA through the detached cotton leaf for 48 hours confirms the presence of dsRNA band in the correct size range (approx. 300 bp).

FIG. 16. Plants-mediated uptake of dsRNA in whitefly B. tabaci. Cy3-labelled CMV 2b dsRNA with (a) and without penetrant (b) were pipetted on the abaxial or adaxial surface of the cotton leaf at a final concentration of 100 ng/μl and allowed to dry for 24 h. Adult whiteflies were released into the clip cage on the abaxial surface and allowed feeding for 48 h. Confocal microscopy of whole insect indicates localization of Cy3-labelled dsRNA in the abdomen of the whitefly, whereas control whiteflies fed on control (water) plant did not show fluorescent signal. Bright-field (BF) image (column 1), Cy3 florescence image (column 2) and merged image of the two (column 3) are shown.

FIG. 17. Whitefly uptake topically applied dsRNA via intact cotton plants. Combination of the 3D surface rendering of the reflective and the fluorescence mode clearly showing the presence of Cy3-labelled CMV 2b dsRNA inside the adult whitefly body.

### DETAILED DESCRIPTION

Before describing the present invention in detail, it is to be understood that the terminology used herein is for the purpose of describing particular embodiments only and is not intended to be limiting.

As used in this specification and the appended claims, the singular forms “a”, “an” and “the” include plural referents unless the content clearly dictates otherwise. Thus, for example, reference to “a binding partner” includes a combination of two or more such binding partners.

Throughout the description and claims of the specification, the word “comprise” and variations of the word, such as “comprising” and “comprises”, is not intended to exclude other additives, components, integers or steps.

Unless specifically defined otherwise, all technical and scientific terms used herein shall be taken to have the same meaning as commonly understood by one of ordinary skill in the art

All publications or patent applications or patents cited herein are entirely incorporated herein by reference.

A reference herein to a publication or patent application or patent or other matter which is given as prior art is not to be taken as admission that the document or matter (e.g., databases) was known or that the information it contains was part of the common general knowledge as at the priority date of any of the claims.

Methods and compositions are provided which employ one or more silencing elements that, when ingested by or contacted with an insect plant pest, such as Bemisia tabaci including, for example, Cassava whitefly, Cotton whitefly, Sliver-leaf whitefly, Sweet-potato whitefly, Tobacco whitefly; B. argentifolii Bellows & Perring; Dialeurodes citri Ashmead Trialeurodes abutiloneus, or T. vaporariorum Westwood plant pest, are capable of decreasing the level of one or more target sequences in the pest.

Disclosed herein are target genes as set forth in SEQ ID NOS 1-35. Silencing elements comprising sequences complementary to a RNA transcript of these sequences, or fragments or variants of these target genes are provided which, when ingested by or when contacting the pest, decrease the expression of one or more of the target sequences and thereby control the pest.

Also disclosed herein are target polynucleotides as set forth in SEQ ID NOS: 36-104, or variants and fragments thereof, and complements thereof. Silencing elements comprising sequences complementary to these sequences, or fragments or variants of these target polynucleotides are provided which, when ingested by or when contacting the pest, decrease the expression of one or more of the target sequences and thereby control the pest.

Also disclosed herein are target polynucleotides encoding proteins set forth in SEQ ID NO:105-138.

Also disclosed herein are silencing elements comprising polynucleotide sequences as set forth in SEQ ID NOS: 139-173, or fragments or variants of these sequences are provided which, when ingested by or when contacting the pest, decrease the expression of one or more of the target sequences and thereby control the pest.

In some embodiments, a formulation (e.g., a sprayable insecticide) comprising one or more polynucleotides encoding one or more silencing elements is provided which, when ingested by or when contacting the pest, decrease the level of one or more of the target sequences and thereby controls the pest.

In other embodiments, a transgenic plant comprising one or more polynucleotides encoding one or more silencing elements is provided which, when ingested by or when contacting the pest, decrease the level of one or more of the target sequences and thereby controls the pest.

In one embodiment, compositions and methods are provided which employ a silencing element comprising at least one double-stranded RNA region, at least one strand of which comprises a polynucleotide that is complementary to:
(a) a nucleotide sequence comprising a sequence of an RNA transcript expressed in an insect plant pest, or variants, isoforms and fragments of said nucleotide sequence, or complements of said nucleotide sequence;
(b) a nucleotide sequence comprising at least 70% sequence identity to said nucleotide sequence; or variants, isoforms and fragments of said nucleotide sequence, or complements of said nucleotide sequence; or
(c) a nucleotide sequence comprising at least 19 consecutive nucleotides of said nucleotide sequence; or variants, isoforms and fragments of said nucleotide sequence, or complements of said nucleotide sequence,
wherein the silencing element has insecticidal activity against an insect plant pest.

In some embodiments, the nucleotide sequence comprises a sequence that is complementary to an RNA transcript of one or more of the following target genes: BtGAP (Bta07742; Hunchback; SEQ ID NO:1), BtTreT (Bta13849; Solute carrier family 2, facilitated glucose transporter member 8; SEQ ID NO:2), BtCOPB1 (Bta11961; Coatomer subunit beta; SEQ ID NO:3), BtAlGluc (Bta07452; Alpha-glucosidase; SEQ ID NO:4), BtCHM4C (Bta01993; Charged multivesicular body protein 4C, putative; SEQ ID NO:5), BtATP synthase (Bta09165; ATP synthase subunit f, mitochondrial; SEQ ID NO:6), BtN2B (Bta20011; Putative ATPase N2B; SEQ ID NO:7), BtRieske (Bta11991; Cytochrome b-c1 complex subunit Rieske, mitochondrial; SEQ ID NO:8), BtaCMB7 (Bta09904; Charged multivesicular body protein 7; SEQ ID NO:9), BtaChorion (Bta13877; Chorion-specific transcription factor NO: 11), BtaEH (Bta11007; EH domain-containing protein 1; SEQ ID NO:12), BtaCleavage (Bta06507; Cleavage stimulation factor subunit 3; SEQ ID NO:13), BtaSyntax (Bta06944; Syntaxin binding protein-1,2,3; SEQ ID NO:14), BtaCd6 (Bta11395; cell division control protein 6 (Cdc6); SEQ ID NO:15), BtaMediator (Bta08552; Mediator of RNA polymerase II transcription subunit 17; SEQ ID NO:16), BtaATP binding (Bta10956; ATP-binding cassette; SEQ ID NO:17), BtaRRP42 (Bta03733; Exosome complex component RRP42; SEQ ID NO:18), BtaKappaB (Bta08258; Nuclear factor kappa-B-binding-like protein; SEQ ID NO:19), BtaATPaseAAA (Bta14342; ATPase family AAA domain-containing protein 1-B; SEQ ID NO: 20) BtarBAT (Bta07377; Neutral and basic amino acid transport protein rBAT; SEQ ID NO:21); BtAChE1 (Bta05381; Acetylcholinesterase 1; SEQ ID NO:22), BtTreH (Bta00329; Trehalase; SEQ ID NO:23), BtAqp1 (Bta01973; Aquaporin 1; SEQ ID NO: 24), Btv-ATPase A (Bta13958; V-type proton ATPase subunit a; SEQ ID NO: 25), BtZFP (Bta11919; Zinc finger protein; SEQ ID NO:26), BtGD (Bta02405; Glucose dehydrogenase; SEQ ID NO:27), BtMYO (Bta07326; Myosin regulatory light chain 2; SEQ ID NO:28), BtDUOX (Bta10996; Dual oxidase; SEQ ID NO:29), BtSUC (Bta14312; Sucrase; SEQ ID NO:30), BtaDyn (Bta02194; Dynactin subunit; SEQ ID NO: 31), BtaNucII (Bta06243; Deoxyribonuclease I (Nuclease II); SEQ ID NO:32), BtTryp_SPc (Bta03794; Trypsin-like serine protease; SEQ ID NO:33), BtPNLIPRP2 (Bta09442; Pancreatic lipase-related protein 2; SEQ ID NO:34), and BtaGTF (Bta07245, Glycosyl transferase; SEQ ID NO:35).

In other embodiments, the nucleotide sequence comprises a sequence that is complementary to an RNA transcript of one or more of the following target genes: BtGAP (Bta07742; Hunchback; SEQ ID NO:1), BtTreT (Bta13849; Solute carrier family 2, facilitated glucose transporter member 8; SEQ ID NO:2), BtCOPB1 (Bta11961; Coatomer subunit beta; SEQ ID NO:3), BtAlGluc (Bta07452; Alpha-glucosidase; SEQ ID NO:4), BtCHM4C (Bta01993; Charged multivesicular body protein 4C, putative; SEQ ID NO:5), BtATP synthase (Bta09165; ATP synthase subunit f, mitochondrial; SEQ ID NO:6), BtN2B (Bta20011; Putative ATPase N2B; SEQ ID NO:7), BtRieske (Bta11991; Cytochrome b-c1 complex subunit Rieske, mitochondrial; SEQ ID NO:8), BtaCMB7 (Bta09904; Charged multivesicular body protein 7; SEQ ID NO:9), BtaChorion (Bta13877; Chorion-specific transcription factor NO: 11), BtaEH (Bta11007; EH domain-containing protein 1; SEQ ID NO:12), BtaCleavage (Bta06507; Cleavage stimulation factor subunit 3; SEQ ID NO:13), BtaSyntax (Bta06944; Syntaxin binding protein-1,2,3; SEQ ID NO:14), BtaCd6 (Bta11395; cell division control protein 6 (Cdc6); SEQ ID NO:15), BtaMediator (Bta08552; Mediator of RNA polymerase II transcription subunit 17; SEQ ID NO:16), BtaATP binding (Bta10956; ATP-binding cassette; SEQ ID NO:17), BtaRRP42 (Bta03733; Exosome complex component RRP42; SEQ ID NO:18), BtaKappaB (Bta08258; Nuclear factor kappa-B-binding-like protein; SEQ ID NO:19), BtaATPaseAAA (Bta14342; ATPase family AAA domain-containing protein 1-B; SEQ ID NO: 20), and BtarBAT (Bta07377; Neutral and basic amino acid transport protein rBAT; SEQ ID NO:21); and BtaGTF (Bta07245, Glycosyl transferase; SEQ ID NO:35).

In further or alternative embodiments, the nucleotide sequence comprises a sequence that is complementary to an RNA transcript of one or more of the following target genes: BtAChE1 (Bta05381; Acetylcholinesterase 1; SEQ ID NO:22), BtTreH (Bta00329; Trehalase; SEQ ID NO:23), BtAqp1 (Bta01973; Aquaporin 1; SEQ ID NO: 24), Btv-ATPase A (Bta13958; V-type proton ATPase subunit a; SEQ ID NO: 25), BtZFP (Bta11919; Zinc finger protein; SEQ ID NO:26), BtGD (Bta02405; Glucose dehydrogenase; SEQ ID NO:27), BtMYO (Bta07326; Myosin regulatory light chain 2; SEQ ID NO:28), BtDUOX (Bta10996; Dual oxidase; SEQ ID NO:29), BtSUC (Bta14312; Sucrase; SEQ ID NO:30), BtaDyn (Bta02194; Dynactin subunit; SEQ ID NO: 31), and BtaNucII (Bta06243; Deoxyribonuclease I (Nuclease II); SEQ ID NO:32).

In further or alternative embodiments, the nucleotide sequence comprises a sequence that is complementary to an RNA transcript of one or both of the following target genes: BtTryp_SPc (Bta03794; Trypsin-like serine protease; SEQ ID NO:33), and BtPNLIPRP2 (Bta09442; Pancreatic lipase-related protein 2; SEQ ID NO:34).

In some embodiments, the nucleotide sequence comprises a sequence that is complementary to one or more of the following RNA transcripts: BtGAP (XM_019041638.1, Hunchback; SEQ ID NO:36), BtTreT (XM_019047819.1, Solute carrier family 2, facilitated glucose transporter member 8; SEQ ID NO:37), BtCOPB1 (XM_019045272.1; Coatomer subunit beta; SEQ ID NO:38), BtAlGluc (XM_019054927.1; Alpha-glucosidase; SEQ ID NO:39), BtCHM4C (XM_019050890.1; Charged multivesicular; body protein 4C, putative; SEQ ID NO: 40), BtATP synthase (XM_019060989.1; ATP synthase subunit f, mitochondrial; SEQ ID NO:41), BtN2B (XM_019043095.1; Putative ATPase N2B; SEQ ID NO:42), BtRieske (XM_019045249.1; Cytochrome b-c1 complex subunit Rieske, mitochondrial; SEQ ID NO:43), BtaCMB7 (XM_019043570.1; Charged multivesicular body protein 7; SEQ ID NO:44), BtaChorion (XM_019047862.1; Chorion-specific transcription factor GCMa, hybrid signal transduction histidine kinase D-like; SEQ ID NO: 45), BtaSurfeit (XM_019049766.1; Surfeit locus protein 4; SEQ ID NO:46), BtaEH (XM_019056878.1; EH domain-containing protein 1; SEQ ID NO:47), BtaCleavage (XM_019053966.1; Cleavage stimulation factor subunit 3, protein suppressor of forked; SEQ ID NO:48), BtaSyntax XM_019040883.1; Syntaxin binding protein-1,2,3, protein ROP; SEQ ID NO:49), BtaCd6 (XM_019044939.1; cell division control protein 6 (Cdc6); SEQ ID NO:50), BtaMediator (XM_019042377.1; Mediator of RNA polymerase II transcription subunit 17; SEQ ID NO:51), BtaATP binding (XM_019044610.1; ATP-binding cassette; SEQ ID NO:52), BtaRRP42 (XM_019061639.1; Exosome complex component RRP42; SEQ ID NO:53), BtaKappaB (XM_019041919.1; Nuclear factor kappa-B-binding-like protein; SEQ ID NO: 54), BtaATPaseAAA (XM_019058468.1; ATPase family AAA domain-containing protein 1-B; SEQ ID NO:55); BtarBAT (XM_019054773.1; Neutral and basic amino acid rBAT; SEQ ID NO:56); BtAChE1 (XM_019040189.1; Acetylcholinesterase 1; SEQ ID NO:57), BtTreH (XM_019049052.1; Trehalase; SEQ ID NO:58), BtAqp1 (XM_019051047.1; Aquaporin 1; SEQ ID NO:59), Btv-ATPase A (XM_019047962.1; V-type proton ATPase subunit a; SEQ ID NO:60); BIZFP (Bta11919; Zinc finger protein), BtGD (XM_019046154.1; Glucose dehydrogenase; SEQ ID NO:61), BtMYO (XM_019041179.1; Myosin regulatory light chain 2; SEQ ID NO:62), BtDUOX (XM_019056938.1; Dual oxidase; SEQ ID NO:63), BtSUC (XM_019048374.1; Sucrase; SEQ ID NO:64), BtaDyn (XM_019045200.1; Dynactin subunit; SEQ ID NO: 65), BtaNucII (XM_019053573.1; Deoxyribonuclease I; SEQ ID NO:66); BtTryp_SPc (XM_019061756.1; Trypsin-like serine protease, venom protease-like; SEQ ID NO:67), BtPNLIPRP2 (XM_019043265.1; Pancreatic lipase-related protein 2; SEQ ID NO:68), and BtaGTF (XM_019041198.1, Glycosyl transferase; SEQ ID NO: 69).

In other embodiments, the nucleotide sequence comprises a sequence that is complementary to one or more of the following RNA transcripts: BtGAP (XM_019041638.1, Hunchback; SEQ ID NO:36), BtTreT (XM_019047819.1, Solute carrier family 2, facilitated glucose transporter member 8; SEQ ID NO:37), BtCOPB1 (XM_019045272.1; Coatomer subunit beta; SEQ ID NO:38), BtAlGluc (XM_019054927.1; Alpha-glucosidase; SEQ ID NO:39), BtCHM4C (XM_019050890.1; Charged multivesicular body protein 4C, putative; SEQ ID NO: 40), BtATP synthase (XM_019060989.1; ATP synthase subunit f, mitochondrial; SEQ ID NO:41), BtN2B (XM_019043095.1; Putative ATPase N2B; SEQ ID NO:42), BtRieske (XM_019045249.1; Cytochrome b-c1 complex subunit Rieske, mitochondrial; SEQ ID NO:43), BtaCMB7 (XM_019043570.1; Charged multivesicular body protein 7; SEQ ID NO:44), BtaChorion (XM_019047862.1; Chorion-specific transcription factor GCMa; SEQ ID NO:45), BtaSurfeit (XM_019049766.1; Surfeit locus protein 4; SEQ ID NO:46), BtaEH (XM_019056878.1; EH domain-containing protein 1; SEQ ID NO:47), BtaCleavage (XM_019053966.1; Cleavage stimulation factor subunit 3; SEQ ID NO:48), BtaSyntax XM_019040883.1; Syntaxin binding protein-1,2,3; SEQ ID NO:49), BtaCd6 (XM_019044939.1; cell division control protein 6 (Cdc6); SEQ ID NO:50), BtaMediator (XM_019042377.1; Mediator of RNA polymerase II transcription subunit 17; SEQ ID NO:51), BtaATP binding (XM_019044610.1; ATP-binding cassette; SEQ ID NO:52), BtaRRP42 (XM_019061639.1; Exosome complex component RRP42; SEQ ID NO:53), BtaKappaB (XM_019041919.1; Nuclear factor kappa-B-binding-like protein; SEQ ID NO: 54), BtaATPaseAAA (XM_019058468.1; ATPase family AAA domain-containing protein 1-B; SEQ ID NO:55), and BtarBAT (XM_019054773.1; Neutral and basic amino acid transport protein rBAT; SEQ ID NO:56), and BtaGTF (XM_019041198.1, Glycosyl transferase; SEQ ID NO:69).

In further or alternative embodiments, the nucleotide sequence comprises a sequence that is complementary to one or more of the following RNA transcripts BtAChE1 (XM_019040189.1; Acetylcholinesterase 1; SEQ ID NO:57), BtTreH (XM_019049052.1; Trehalase; SEQ ID NO:58), BtAqp1 (XM_019051047.1; Aquaporin 1; SEQ ID NO:59), Btv-ATPase A (XM_019047962.1; V-type proton ATPase subunit a; SEQ ID NO:60); BIZFP (Zinc finger protein), BtGD (XM_019046154.1; Glucose dehydrogenase; SEQ ID NO:61), BtMYO (XM_019041179.1; Myosin regulatory light chain 2; SEQ ID NO:62), BtDUOX (XM_019056938.1; Dual oxidase; SEQ ID NO:63), BtSUC (XM_019048374.1; Sucrase; SEQ ID NO:64), BtaDyn (XM_019045200.1; Dynactin subunit; SEQ ID NO: 65), BtaNucII (XM_019053573.1; Deoxyribonuclease I (Nuclease II); SEQ ID NO: 66).

In further or alternative embodiments, the nucleotide sequence comprises a sequence that is complementary to one or both of the following RNA transcripts BtTryp_SPc (XM_019061756.1; Trypsin-like serine protease; SEQ ID NO:67) and BtPNLIPRP2 (XM_019043265.1; Pancreatic lipase-related protein 2; SEQ ID NO:68).

In other embodiments, the nucleotide sequence comprises a sequence that is complementary to one or more of the following RNA transcripts: BtGAP (Bta07742; Hunchback; SEQ ID NO:70), BtTreT (Bta13849; Solute carrier family 2, facilitated glucose transporter member 8; SEQ ID NO:71), BtCOPB1 (Bta11961; Coatomer subunit beta; SEQ ID NO:72), BtAlGluc (Bta07452; Alpha-glucosidase; SEQ ID NO: 73), BtCHM4C (Bta01993; Charged multivesicular body protein 4C, putative; SEQ ID NO: 74), BtATP synthase (Bta09165; ATP synthase subunit f, mitochondrial; SEQ ID NO: 75), BtN2B (Bta20011; Putative ATPase N2B; SEQ ID NO:76), BtRieske (Bta11991; Cytochrome b-c1 complex subunit Rieske, mitochondrial; SEQ ID NO:77), BtaCMB7 (Bta09904; Charged multivesicular body protein 7; SEQ ID NO:78), BtaChorion (Bta13877; Chorion-specific transcription factor GCMa; SEQ ID NO:79), BtaSurfeit (Bta01046; Surfeit locus protein 4; SEQ ID NO:80), BtaEH (Bta11007; EH domain-containing protein 1; SEQ ID NO:81), BtaCleavage (Bta06507; Cleavage stimulation factor subunit 3; SEQ ID NO:82), BtaSyntax (Bta06944; Syntaxin binding protein-1,2,3; SEQ ID NO:83), BtaCd6 (Bta11395; cell division control protein 6 (Cdc6); SEQ ID NO:84), BtaMediator (Bta08552; Mediator of RNA polymerase II transcription subunit 17; SEQ ID NO:85), BtaATP binding (Bta10956; ATP-binding cassette; SEQ ID NO:86), BtaRRP42 (Bta03733; Exosome complex component RRP42; SEQ ID NO:87), BtaKappaB (Bta08258; Nuclear factor kappa-B-binding-like protein; SEQ ID NO:88), BtaATPaseAAA (Bta14342; ATPase family AAA domain-containing protein 1-B; SEQ ID NO:89), BtarBAT (Bta07377; Neutral and basic amino acid transport protein rBAT; SEQ ID NO:90), BtAChE1 (Bta05381; Acetylcholinesterase 1; SEQ ID NO:91), BtTreH (Bta00329; Trehalase; SEQ ID NO: 92), BtAqp1 (Bta01973; Aquaporin 1; SEQ ID NO:93), Btv-ATPase A (Bta13958; V-type proton ATPase subunit a; SEQ ID NO:94); BIZFP (Bta11919; Zinc finger protein; SEQ ID NO:95), BtGD (Bta02405; Glucose dehydrogenase; SEQ ID NO:96), BtMYO (Bta07326; Myosin regulatory light chain 2; SEQ ID NO:97), BtDUOX (Bta10996; Dual oxidase; SEQ ID NO:98), BtSUC (Bta14312; Sucrase; SEQ ID NO: 99), BtaDyn (Bta02194; Dynactin subunit; SEQ ID NO:100), and BtaNucII (Bta06243; Deoxyribonuclease I (Nuclease II); SEQ ID NO:101), BtTryp_SPc (Bta03794; Trypsin-like serine protease; SEQ ID NO:102), BtPNLIPRP2 (Bta09442; Pancreatic lipase-related protein 2; SEQ ID NO:103), and BtaGTF (Bta07245; Glycosyl transferase; SEQ ID NO:104).

In other embodiments, the nucleotide sequence comprises a sequence that is complementary to one or more of the following RNA transcripts: BtGAP (Bta07742; Hunchback; SEQ ID NO:70), BtTreT (Bta13849; Solute carrier family 2, facilitated glucose transporter member 8; SEQ ID NO: 71), BtCOPB1 (Bta1 1961; Coatomer subunit beta; SEQ ID NO:72), BtAlGluc (Bta07452; Alpha-glucosidase; SEQ ID NO: 73), BtCHM4C (Bta01993; Charged multivesicular body protein 4C, putative; SEQ ID NO: 74), BtATP synthase (Bta09165; ATP synthase subunit f, mitochondrial; SEQ ID NO: 75), BtN2B (Bta20011; Putative ATPase N2B; SEQ ID NO:76), BtRieske (Bta11991; Cytochrome b-c1 complex subunit Rieske, mitochondrial; SEQ ID NO:77), BtaCMB7 (Bta09904; Charged multivesicular body protein 7; SEQ ID NO:78), BtaChorion (Bta13877; Chorion-specific transcription factor GCMa; SEQ ID NO:79), BtaSurfeit (Bta01046; Surfeit locus protein 4; SEQ ID NO:80), BtaEH (Bta11007; EH domain-containing protein 1; SEQ ID NO:81), BtaCleavage (Bta06507; Cleavage stimulation factor subunit 3; SEQ ID NO:82), BtaSyntax (Bta06944; Syntaxin binding protein-1,2,3; SEQ ID NO:83), BtaCd6 (Bta11395; cell division control protein 6 (Cdc6); SEQ ID NO:84), BtaMediator (Bta08552; Mediator of RNA polymerase II transcription subunit 17; SEQ ID NO:85), BtaATP binding (Bta10956; ATP-binding cassette; SEQ ID NO:86), BtaRRP42 (Bta03733; Exosome complex component RRP42; SEQ ID NO:87), BtaKappaB (Bta08258; Nuclear factor kappa-B-binding-like protein; SEQ ID NO:88), BtaATPaseAAA (Bta14342; ATPase family AAA domain-containing protein 1-B; SEQ ID NO:89); BtarBAT (Bta07377; Neutral and basic amino acid transport protein rBAT; SEQ ID NO:90), and BtaGTF (Bta07245; Glycosyl transferase; SEQ ID NO:104).

In further or alternative embodiments, the nucleotide sequence comprises a sequence that is complementary to one or more of the following RNA transcripts: BtAChE1 (Bta05381; Acetylcholinesterase 1; SEQ ID NO:91), BtTreH (Bta00329; Trehalase; SEQ ID NO:92), BtAqp1 (Bta01973; Aquaporin 1; SEQ ID NO:93), Btv-ATPase A (Bta13958; V-type proton ATPase subunit a; SEQ ID NO:94); BtZFP (Bta11919; Zinc finger protein; SEQ ID NO:95), BtGD (Bta02405; Glucose dehydrogenase; SEQ ID NO:96), BtMYO (Bta07326; Myosin regulatory light chain 2; SEQ ID NO:97), BtDUOX (Bta10996; Dual oxidase; SEQ ID NO:98), BtSUC (Bta14312; Sucrase; SEQ ID NO:99), BtaDyn (Bta02194; Dynactin subunit; SEQ ID NO: 100), and BtaNucII (Bta06243; Deoxyribonuclease I (Nuclease II); SEQ ID NO: 101).

In further or alternative embodiments, the nucleotide sequence comprises a sequence that is complementary to one or both of the following RNA transcripts: BtTryp_SPc (Bta03794; Trypsin-like serine protease; SEQ ID NO:102) and BtPNLIPRP2 (Bta09442; Pancreatic lipase-related protein 2; SEQ ID NO:103).

In some embodiments, the nucleotide sequence comprises a sequence that is complementary to an RNA transcript of one or more of the following target genes: BtGAP (Bta07742; Hunchback; SEQ ID NO:1), BtCOPB1 (Bta11961; Coatomer subunit beta; SEQ ID NO:3), BtCHM4C (Bta01993; Charged multivesicular body protein 4C, putative; SEQ ID NO:5), BtN2B (Bta20011; Putative ATPase N2B; SEQ ID NO:7), BtRieske (Bta11991; Cytochrome b-c1 complex subunit Rieske, mitochondrial; SEQ ID NO:8), BtaCMB7 (Bta09904; Charged multivesicular body protein 7; SEQ ID NO:9), BtaChorion (Bta13877; Chorion-specific transcription factor GCMa; SEQ ID NO:10), BtaSurfeit; (Bta01046; Surfeit locus protein 4; SEQ ID NO: 11), BtaEH (Bta11007; EH domain-containing protein 1; SEQ ID NO:12), BtaCleavage (Cleavage stimulation factor subunit 3; SEQ ID NO:13), BtaSyntax (Bta06944; Syntaxin binding protein-1,2,3; SEQ ID NOL14), BtaCd6 (Bta11395; Cdc6; SEQ ID NO:15), BtaMediator (Bta08552; Mediator of RNA polymerase II transcription subunit 17; SEQ ID NO:16), BtaATP binding (Bta10956; ATP-binding cassette; SEQ ID NO:17), BtaRRP42 (Bta03733; Exosome complex component RRP42; SEQ ID NO:18), BtaKappaB (Bta08258; Nuclear factor kappa-B-binding-like protein; SEQ ID NO:19), and BtaATPaseAAA (Bta14342; ATPase family AAA domain-containing protein 1-B; SEQ ID NO:20), BtZFP (Bta11919; Zinc finger protein; SEQ ID NO:26), BtGD (Bta02405; Glucose dehydrogenase; SEQ ID NO:27), BtMYO (Bta07326; Myosin regulatory light chain 2; SEQ ID NO:28), BtDUOX (Bta10996; Dual oxidase; SEQ ID NO:29), BtSUC (Bta14312; Sucrase; SEQ ID NO:30), BtaDyn (Bta02194; Dynactin subunit; SEQ ID NO:31), BtTryp_SPc (Bta03794; Trypsin-like serine protease; SEQ ID NO:33), and BtPNLIPRP2 (Bta09442; Pancreatic lipase-related protein 2; SEQ ID NO:34).

In some embodiments, the nucleotide sequence comprises a sequence that is complementary to one or more of the following RNA transcripts: BtGAP (XM_019041638.1; Hunchback; SEQ ID NO:36), BtCOPB1 (XM_019045272.1; Coatomer subunit beta; SEQ ID NO:38), BtCHM4C (XM_019050890.1; Charged multivesicular body protein 4C, putative; SEQ ID NO:40), BtN2B (XM_019043095.1; Putative ATPase N2B; SEQ ID NO:42), BtRieske (XM_019045249.1; Cytochrome b-c1 complex subunit Rieske, mitochondrial; SEQ ID NO:43), BtaCMB7 (XM_019043570.1; Charged multivesicular body protein 7; SEQ ID NO:44), BtaChorion (XM_019047862.1; Chorion-specific transcription factor GCMa; SEQ ID NO: 45), BtaSurfeit; (XM_019049766.1; Surfeit locus protein 4; SEQ ID NO:46), BtaEH (XM_019056878.1; EH domain-containing protein 1; SEQ ID NO:47), BtaCleavage (XM_019053966.1; Cleavage stimulation factor subunit 3; SEQ ID NO: 48), BtaSyntax (XM_019040883.1; Syntaxin binding protein-1,2,3; SEQ ID NO: 49), BtaCd6 (XM_019044939.1; Cdc6; SEQ ID NO:50), BtaMediator (XM_019042377.1; Mediator of RNA polymerase II transcription subunit 17; SEQ ID NO: 51), BtaATP binding (XM_019044610.1; ATP-binding cassette; SEQ ID NO:52), BtaRRP42 (XM_019061639.1; Exosome complex component RRP42; SEQ ID NO: 53), BtaKappaB (XM_019041919.1; Nuclear factor kappa-B-binding-like protein; SEQ ID NO:54), and BtaATPaseAAA (XM_019058468.1; ATPase family AAA domain-containing protein 1-B; SEQ ID NO:55), BIZFP (Zinc finger protein), BtGD (XM_019046154.1; Glucose dehydrogenase; SEQ ID NO:61), BtMYO (XM_019041179.1; Myosin regulatory light chain 2; SEQ ID NO:62), BtDUOX (XM_019056938.1; Dual oxidase; SEQ ID NO:63), BtSUC (XM_019048374.1; Sucrase; SEQ ID NO:64), BtaDyn (XM_019045200.1; Dynactin subunit; SEQ ID NO: 65), BtTryp_SPc (XM_019061756.1; Trypsin-like serine protease; SEQ ID NO: 67), BtPNLIPRP2 (XM_019043265.1; Pancreatic lipase-related protein 2; SEQ ID NO: 68).

In other embodiments, the nucleotide sequence comprises a sequence that is complementary to one or more of the following RNA transcripts: BtGAP (Bta07742; Hunchback; SEQ ID NO:70), BtCOPB1 (Bta11961; Coatomer subunit beta; SEQ ID NO: 72), BtCHM4C (Bta01993; Charged multivesicular body protein 4C, putative; SEQ ID NO: 74), BtN2B (Bta20011; Putative ATPase N2B; SEQ ID NO:76), BtRieske (Bta11991; Cytochrome b-c1 complex subunit Rieske, mitochondrial; SEQ ID NO:77), BtaCMB7 (Bta09904; Charged multivesicular body protein 7; SEQ ID NO:78), BtaChorion (Bta13877; Chorion-specific transcription factor GCMa; SEQ ID NO:79), BtaSurfeit (Bta01046; Surfeit locus protein 4; SEQ ID NO:80), BtaEH (Bta11007; EH domain-containing protein 1; SEQ ID NO:81), BtaCleavage (Bta06507; Cleavage stimulation factor subunit 3; SEQ ID NO:82), BtaSyntax (Bta06944; Syntaxin binding protein-1,2,3; SEQ ID NO:83), BtaCd6 (Bta11395; Cdc6; SEQ ID NO:84), BtaMediator (Bta08552; Mediator of RNA polymerase II transcription subunit 17; SEQ ID NO: 85), BtaATP binding (Bta10956; ATP-binding cassette; SEQ ID NO:86), BtaRRP42 (Bta03733; Exosome complex component RRP42; SEQ ID NO:87), BtaKappaB (Bta08258; Nuclear factor kappa-B-binding-like protein; SEQ ID NO:88), and BtaATPaseAAA (Bta14342; ATPase family AAA domain-containing protein 1-B; SEQ ID NO:89), BtZFP (Zinc finger protein), BtGD (Bta02405; Glucose dehydrogenase; SEQ ID NO:96), BtMYO (Bta07326; Myosin regulatory light chain 2; SEQ ID NO:97), BtDUOX (Bta10996; Dual oxidase; SEQ ID NO:98), BtSUC (Bta14312; Sucrase; SEQ ID NO:99), BtaDyn (Bta02194; Dynactin subunit; SEQ ID NO: 100), BtTryp_SPc (Bta03794; Trypsin-like serine protease; SEQ ID NO:102), BtPNLIPRP2 (Bta09442; Pancreatic lipase-related protein 2; SEQ ID NO:103).

In another embodiment, compositions and methods are provided which employ one or more silencing elements which target a polynucleotide encoding one or more of the following proteins: Hunchback (XP_018897183.1; SEQ ID NO:105), Solute carrier family 2, facilitated glucose transporter member 8 (XP_018903364.1; SEQ ID NO:106), Coatomer subunit beta (XP_018900817.1; SEQ ID NO:107), Alpha-glucosidase (XP_018900817.1; SEQ ID NO:108), Charged multivesicular body protein 4C, putative (XP_018906435.1; SEQ ID NO:109), ATP synthase subunit f, mitochondrial (XP_018916534.1; SEQ ID NO:110), Putative ATPase N2B (XP_018898640.1; SEQ ID NO:111), Cytochrome b-c1 complex subunit Rieske, mitochondrial (XP_018900794.1; SEQ ID NO:112), Charged multivesicular body protein 7 (XP_018899115.1; SEQ ID NO:113), Chorion-specific transcription factor GCMa (XP_018903407.1; SEQ ID NO:114), Surfeit locus protein 4 (XP_018905311.1; SEQ ID NO:115), EH domain-containing protein 1 (XP_018912423.1; SEQ ID NO:116), Cleavage stimulation factor subunit 3 (XP_018909511.1; SEQ ID NO:117), Syntaxin binding protein-1,2,3 (XP_018896428.1; SEQ ID NO:118), Cdc6 (XP_018900484.1; SEQ ID NO:119), Mediator of RNA polymerase II transcription subunit 17 (XP_018897922.1; SEQ ID NO: 120), ATP-binding cassette (XP_018900155.1; SEQ ID NO:121), Exosome complex component RRP42 (XP_018917184.1; SEQ ID NO:122), Nuclear factor kappa-B-binding-like protein (XP_018897464.1; SEQ ID NO:123), ATPase family AAA domain-containing protein 1-B (XP_018914013.1; SEQ ID NO:124), Neutral and basic amino acid transport protein rBAT (XP_018910318.1; SEQ ID NO:125), Acetylcholinesterase 1 (XP_018895734.1; SEQ ID NO:126), Trehalase (XP_018904597.1; SEQ ID NO:127), Aquaporin 1 (XP_018906592.1; SEQ ID NO: 128), V-type proton ATPase subunit a (XP_018903507.1; SEQ ID NO:129), Zinc finger protein; Glucose dehydrogenase (XP_018901699.1; SEQ ID NO:130), Myosin regulatory light chain 2 (XP_018896724.1; SEQ ID NO:131), Dual oxidase (XP_018912483.1; SEQ ID NO:132), Sucrase (XP_018903919.1; SEQ ID NO:133), Dynactin subunit (XP_018900745.1; SEQ ID NO:134), Deoxyribonuclease I (Nuclease II) (XP_018909118.1; SEQ ID NO:135), Trypsin-like serine protease (XP_018917301.1; SEQ ID NO:136), Pancreatic lipase-related protein 2 (XP_018898810.1; SEQ ID NO:137), and Glycosyl transferase (XP_018896743.1; SEQ ID NO:138).

In another embodiment, compositions and methods are provided which employ one or more silencing elements which target a polynucleotide encoding one or more of the following proteins: Hunchback (XP_018897183.1; SEQ ID NO:105), Solute carrier family 2, facilitated glucose transporter member 8 (XP_018903364.1; SEQ ID NO:106), Coatomer subunit beta (XP_018900817.1; SEQ ID NO:107), Alpha-glucosidase (XP_018900817.1; SEQ ID NO:108), Charged multivesicular; body protein 4C, putative (XP_018906435.1; SEQ ID NO:109), ATP synthase subunit f, mitochondrial (XP_018916534.1; SEQ ID NO:110), Putative ATPase N2B (XP_018898640.1; SEQ ID NO:111), Cytochrome b-c1 complex subunit Rieske, mitochondrial (XP_018900794.1; SEQ ID NO:112), Charged multivesicular body protein 7 (XP_018899115.1; SEQ ID NO:113), Chorion-specific transcription factor GCMa (XP_018903407.1; SEQ ID NO:114), Surfeit locus protein 4 (XP_018905311.1; SEQ ID NO:115), EH domain-containing protein 1 (XP_018912423.1; SEQ ID NO:116), Cleavage stimulation factor subunit 3 (XP_018909511.1; SEQ ID NO:117), Syntaxin binding protein-1,2,3 (XP_018896428.1; SEQ ID NO:118), Cdc6 (XP_018900484.1; SEQ ID NO:119), Mediator of RNA polymerase II transcription subunit 17 (XP_018897922.1; SEQ ID NO: 120), ATP-binding cassette (XP_018900155.1; SEQ ID NO:121), Exosome complex component RRP42 (XP_018917184.1; SEQ ID NO:122), Nuclear factor kappa-B-binding-like protein (XP_018897464.1; SEQ ID NO:123), ATPase family AAA domain-containing protein 1-B (XP_018914013.1; SEQ ID NO:124), Neutral and basic amino acid transport protein rBAT (XP_018910318.1; SEQ ID NO:125), and Glycosyl transferase (XP_018896743.1; SEQ ID NO:138).

In further or alternative embodiments, compositions and methods are provided which employ one or more silencing elements which target a polynucleotide encoding one or more of the following proteins: Acetylcholinesterase 1 (XP_018895734.1; SEQ ID NO:126), Trehalase (XP_018904597.1; SEQ ID NO:127), Aquaporin 1 (XP_018906592.1; SEQ ID NO:128), V-type proton ATPase subunit a (XP_018903507.1; SEQ ID NO:129), Zinc finger protein; Glucose dehydrogenase (XP_018901699.1; SEQ ID NO:130), Myosin regulatory light chain 2 (XP_018896724.1; SEQ ID NO:131), Dual oxidase (XP_018912483.1; SEQ ID NO: 132); Sucrase (XP_018903919.1; SEQ ID NO:133), Dynactin subunit (XP_018900745.1; SEQ ID NO:134), and Deoxyribonuclease I (Nuclease II) (XP_018909118.1; SEQ ID NO:135).

In further or alternative embodiments, compositions and methods are provided which employ one or more silencing elements which target a polynucleotide encoding one or both of the following proteins: Trypsin-like serine protease (XP_018917301.1; SEQ ID NO:136) and Pancreatic lipase-related protein 2 (XP_018898810.1; SEQ ID NO:137).

In some embodiments, compositions and methods are provided which employ one or more silencing elements which target a polynucleotide encoding one or more of the following proteins: Hunchback (XP_018897183.1; SEQ ID NO:105), Coatomer subunit beta (XP_018900817.1; SEQ ID NO:107), Charged multivesicular body protein 4C, putative (XP_018906435.1; SEQ ID NO:109), Putative ATPase N2B (XP_018898640.1; SEQ ID NO:111), Cytochrome b-c1 complex subunit Rieske, mitochondrial (XP_018900794.1; SEQ ID NO:112), Charged multivesicular body protein 7 (XP_018899115.1; SEQ ID NO:113), Chorion-specific transcription factor GCMa (XP_018903407.1; SEQ ID NO:114), Surfeit locus protein 4 (XP_018905311.1; SEQ ID NO:115), EH domain-containing protein 1 (XP_018912423.1; SEQ ID NO:116), Cleavage stimulation factor subunit 3 (XP_018909511.1; SEQ ID NO:117), Syntaxin binding protein-1,2,3 (XP_018896428.1; SEQ ID NO:118), Cdc6 (XP_018900484.1; SEQ ID NO:119), Mediator of RNA polymerase II transcription subunit 17 (XP_018897922.1; SEQ ID NO: 120), ATP-binding cassette (XM_019044610.1; SEQ ID NO:121), Exosome complex component RRP42 (XM_019061639.1; SEQ ID NO:122), Nuclear factor kappa-B-binding-like protein (XM_019041921.1; SEQ ID NO:123); ATPase family AAA domain-containing protein 1-B (XP_018914013.1; SEQ ID NO:124), Zinc finger protein; Glucose dehydrogenase (XP_018901699.1; SEQ ID NO:130), Myosin regulatory light chain 2 (XP_018896724.1; SEQ ID NO:131), Dual oxidase; Sucrase (XP_018912483.1; SEQ ID NO:132), Dynactin subunit (XP_018900745.1; SEQ ID NO: 134), Trypsin-like serine protease (XP_018917301.1; SEQ ID NO:136), and Pancreatic lipase-related protein 2 (XP_018898810.1; SEQ ID NO:137).

In one embodiment, compositions and methods are provided which employ a silencing element comprising at least one double-stranded RNA region, at least one strand of which comprises a nucleotide sequence comprising any one of SEQ ID NOS: 139-173; or variants and fragments thereof, and complements thereof. Variants and fragments also encode biologically active silencing elements (e.g., insecticidal dsRNAs). The variants may comprise one or more mutations and/or modifications that may for example, confer improved or altered properties on the dsRNAs.

In another embodiment, compositions and methods are provided which employ expression constructs, for example, DNA constructs, or vectors, for example, viral vectors, encoding one or more silencing elements, which when ingested by or contacted with the pest, decrease the level of one or more of the target polynucleotides, and thereby control the pest.

In another embodiment, plants, plant parts, seed, plant cells, and other host cells or organisms comprising the expression constructs, for example, DNA constructs, or vectors, for example, viral vectors, encoding the silencing elements or an active variant or fragment thereof are also provided.

The term “nucleic acid sequence” or “nucleic acid molecule” or polynucleotide are used interchangeably and refer to a DNA or RNA molecule in single or double stranded form.

The term “messenger RNA” or “mRNA” refers to RNA that is transcribed from genomic DNA and that carries the coding sequence for protein synthesis. Pre-mRNA (precursor mRNA) is transcribed from genomic DNA. In eukaryotes, pre-mRNA is processed into mRNA, which includes removal of the introns, i.e., “splicing”, and modifications to 5′ and 3′ end (e.g., polyadenylation). mRNA typically comprises from 5′ to 3′; a 5′cap (modified guanine nucleotide), 5′ UTR (untranslated region), the coding sequence (beginning with a start codon and ending with a stop codon), 3′ UTR, and the poly(A) tail.

As used herein, the terms “precursor mRNA” or “pre-mRNA” refer to an immature single strand of messenger ribonucleic acid (mRNA) that contains one or more intervening sequence(s) (introns). Pre-mRNA is transcribed by an RNA polymerase from a DNA template in the cell nucleus and is comprised of alternating sequences of introns and coding regions (exons). Once a pre-mRNA has been completely processed by the splicing out of introns and joining of exons, it is referred to as “messenger RNA” or “mRNA,” which is an RNA that is comprised exclusively of exons. Eukaryotic pre-mRNAs exist only transiently before being fully processed into mRNA. When a pre-mRNA has been processed to an mRNA sequence, it is exported out of the nucleus and eventually translated into a protein by ribosomes in the cytoplasm.

The term “intron” refers to a portion of a gene that is not translated into protein and while present in genomic DNA and pre-mRNA, it is removed in the formation of mature mRNA.

The term “exon” refers to a portion of a gene that is present in the mature form of mRNA. Exons include the ORF (open reading frame), i.e., the sequence which encodes protein, as well as 5′ and 3′ UTRs (untranslated regions). The UTRs are important for translation of the protein. Algorithms and computer programs are available for predicting exons in DNA sequences and for determining exon-intron junctions).

As used herein, “target mRNA” refers to the nucleic acid molecule to which the silencing elements provided herein are designed to hybridize. In the context of the present disclosure, target mRNA is usually mature mRNA.

As used herein, “hybridization” means the pairing of complementary strands of polynucleotides. In the context of the present disclosure, a polynucleotide is specifically hybridizable when there is a sufficient degree of complementarity to avoid non-specific binding of the polynucleotide to non-target nucleic acid sequences. One of skill in the art will be able to determine when a polynucleotide is specifically hybridizable.

As used herein, “complementary” refers to a nucleic acid molecule that can form hydrogen bond(s) with another nucleic acid molecule by either traditional Watson-Crick base pairing or other non-traditional types of pairing (e.g., Hoogsteen or reversed Hoogsteen hydrogen bonding) between complementary nucleosides or nucleotides. “Complementary” (or “specifically hybridizable”) indicates a sufficient degree of complementarity or precise pairing such that stable and specific binding occurs between a silencing element and a pre-mRNA or mRNA target. It is understood in the art that a nucleic acid molecule need not be 100% complementary to a target nucleic acid sequence to be specifically hybridizable. Complementarity is indicated by a percentage of residues in a nucleic acid molecule that can form hydrogen bonds with a second nucleic acid molecule.

Percent complementarity of a silencing element with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Nucleic acid molecules that are “fully complementary” refers to those in which all the residues of a first nucleic acid molecule will hydrogen bond with the same number of contiguous residues in a second nucleic acid molecule, wherein the nucleic acid molecules either both have the same number of nucleotides (i.e., have the same length) or the two molecules have different lengths.

“Sequence identity” and “sequence similarity” can be determined by alignment of two nucleotide sequences using global or local alignment algorithms. Sequences may then be referred to as “substantially identical” or “essentially similar” when they are optimally aligned by for example the programs GAP or BESTFIT or the Emboss program “Needle” (using default parameters, see below) share at least a certain minimal percentage of sequence identity (as defined further below). These programs use the Needleman and Wunsch global alignment algorithm to align two sequences over their entire length, maximising the number of matches and minimises the number of gaps. Generally, the default parameters are used, with a gap creation penalty=10 and gap extension penalty=0.5 (both for nucleotide and protein alignments). For nucleotides the default scoring matrix used is DNAFULL (Henikoff & Henikoff, 1992, PNAS 89, 10915-10919). Sequence alignments and scores for percentage sequence identity may for example be determined using computer programs, such as EMBOSS (http://www.ebi.ac.uk/Tools/psa/emboss_needle/). Alternatively sequence similarity or identity may be determined by searching against databases such as FASTA, BLAST, etc., but hits should be retrieved and aligned pairwise to compare sequence identity. Two nucleic acid sequences have “substantial sequence identity” if the percentage sequence identity is at least 70%, 75%, 80%, 85%, 90%, 95%, 98%, 99% or more, preferably 90%, 95%, 98%, 99% or more (as determined by, for example, Emboss “needle” using default parameters, i.e. gap creation penalty=10, gap extension penalty=0.5, using scoring matrix DNAFULL for nucleic acids). Such sequences are also referred to as ‘variants’ herein. It should be understood that sequences with substantial sequence identity do not necessarily have the same length and may differ in length. For example, sequences that have the same nucleotide sequence but have additional nucleotides on 3′- and/or 5′-side are 100% identical.

A “fragment” of a polynucleotide refers to any subset of the molecule, i.e., a shorter polynucleotide.

A “variant” refers to a molecule substantially similar to the polynucleotide, such as a nucleotide substitution variant having one or more substituted nucleotides, but which maintains the ability to hybridize with the target sequence. Variants also include longer sequences.

As used herein, by “controlling a plant insect pest” or “controls a plant insect pest” is intended any effect on a plant insect pest that results in limiting the damage that the pest causes. Controlling a plant insect pest includes, but is not limited to, killing the pest, inhibiting development of the pest, altering fertility or growth of the pest in such a manner that the pest provides less damage to the plant, or in a manner for decreasing the number of offspring produced, producing less fit pests, including offspring, producing pests more susceptible to predator attack, producing pests more susceptible to other insecticides, decreasing pathogen (e.g., viral) transmission, or deterring the pests from eating the plant.

Reducing the level of the target polynucleotide or the polypeptide encoded thereby, in the pest results in suppression, control, and/or killing the pest. In one embodiment, reducing the level of the target sequence of the pest will reduce the pest damage by at least about 2% to at least about 6%, at least about 5% to about 50%, at least about 10% to about 60%, at least about 30% to about 70%, at least about 40% to about 80%, or at least about 50% to about 90% or greater. Hence, methods disclosed herein can be utilized to control pests, including but not limited to Bemisia tabaci; B. argentifolii Bellows & Perring; Dialeurodes citri Ashmead Trialeurodes abutiloneus, or T. vaporariorum Westwood plant pests.

Certain assays measuring the control of an insect plant pest are commonly known in the art, for example, artificial diet assay, detached leaf or petiole dip assay, vegetable soaking, foliar spray.

Disclosed herein are compositions and methods for protecting plants from an insect plant pest, or inducing resistance in a plant to an insect plant pest, such as Bemisia tabaci; B. argentifolii Bellows & Perring; Dialeurodes citri Ashmead Trialeurodes abutiloneus, or T. vaporariorum Westwood plant pest, are capable of decreasing the expression of a target sequence in the pest. In some embodiments, the insect plant pest is Bemisia tabaci of the order Hemiptera. There are currently 37 recognized Bemisia tabaci cryptic species (see, https:/www.nature.com/articles/s41598-019-42793-8). In one embodiment, the insect plant pest is Bemisia tabaci MEAM-1 or Biotype B.

### Target Sequences

As used herein, a “target sequence” or “target polynucleotide” comprises any sequence, partial or full-length, of a gene in the pest that one desires to reduce the level of expression thereof. In certain embodiments, decreasing the level of the target sequence (e.g., mRNA sequence) in the pest controls the pest. For example, the target sequence may be essential for growth and development. Non-limiting examples of target sequences include a polynucleotide comprising any one of SEQ ID NOS: 36-104, or variants and fragments of said nucleotide sequence, or complements of said nucleotide sequence. As exemplified elsewhere herein, decreasing the level of one or more of these target sequences in a Bemisia tabaci plant pest controls the pest. In one embodiment, a target sequence encodes a protein necessary for insect fitness and may be involved in sugar metabolism and/or excretion, ion transport, growth or development. In preferred embodiments, the target sequence is mRNA or pre-mRNA.

### Silencing Elements

As used herein, a “silencing element” is a polynucleotide which when ingested by or contacted with a plant insect pest, is capable of reducing the level of a target polynucleotide or the polypeptide encoded thereby to suppress, control, and/or kill the pest. Accordingly, it is to be understood that “silencing element” as used herein, comprises polynucleotides such as RNA constructs, double stranded RNA (dsRNA), hairpin RNA, siRNA, miRNA, amiRNA, and antisense RNA.

In preferred embodiments, the silencing element has insecticidal activity. By “insecticidal activity” it is meant that the silencing element supresses, controls, and/or kills the pest. In some embodiments, the pest mortality rate of the silencing element is at least about 10%, at least about 15%, at least about 20%, at least about 25%, at least about 30%, at least about 35%, at least about 40%, at least about 50%, at least about 55%, at least about 60%, at least about 70%, at least about 80%. The pest mortality rate may refer to the mortality rate or one or more of adult pests, nymph or eggs.

The silencing element employed can reduce the level of the target sequence by influencing the level of the target RNA transcript or, alternatively, by influencing translation and thereby affecting the level of the encoded polypeptide. Methods to assay for functional silencing elements that are capable of reducing the level of a target sequence are disclosed elsewhere herein.

A single polynucleotide employed in the disclosed methods can comprise one or more silencing elements to the same or different target polynucleotides. The silencing element can be produced in vivo (e.g., in a host such as a plant or microorganism) or in vitro (e.g., in a host cell or cell free expression system).

In certain embodiments, a silencing element may be chimeric comprising two or more disclosed sequences or fragments thereof. For example, the chimera may be a dsRNA as disclosed herein. In one embodiment, the chimera comprises two complementary sequences disclosed herein, or fragments thereof, having some degree of mismatch between the complementary sequences such that the two sequences are not perfect complements of one another. Providing at least two different sequences in a single silencing element may allow for targeting multiple genes using one silencing element and/or for example, one expression cassette. Targeting multiple genes may allow for slowing or reducing the possibility of resistance by the pest. In addition, providing multiple targeting ability in one expressed molecule may reduce the expression burden of the transformed plant or plant product, or provide topical treatments that are capable of targeting multiple pests with one application.

The silencing element can comprise additional sequences that advantageously effect transcription and/or the stability of a resulting transcript. As discussed in further detail below, enhancer suppressor elements can also be employed in conjunction with the silencing elements disclosed herein.

In certain embodiments, while the silencing element supresses, controls or kills pests, preferably the silencing element has no effect on the plant or plant part.

As used herein “reduces” or “reducing” the level of a polynucleotide or a polypeptide encoded thereby is intended to mean, the polynucleotide or polypeptide level of the target sequence is statistically lower than the polynucleotide level or polypeptide level of the same target sequence in an appropriate control pest which is not exposed to (i.e., has not ingested or come into contact with) the silencing element. In particular embodiments, methods and/or compositions disclosed herein reduce the polynucleotide level and/or the polypeptide level of the target sequence in a plant insect pest to less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control pest.

In some embodiments, a silencing element has substantial sequence identity to the target polynucleotide, typically greater than about 65% sequence identity, greater than about 70% sequence identity, greater than about 80% sequence identity, greater than about 85% sequence identity, about 90%, 91%, 92%, 93%, 94%, 95%, 96%, 97%, 98%, or 99% sequence identity. Furthermore, a silencing element can be complementary to a part of the target polynucleotide. Generally, target sequences of at least 15, 16, 17, 18, 19, 20, 22, 25, 50, 100, 200, 300, 400, 450 continuous nucleotides or greater of the sequence of any of SEQ ID NOS: 36-104, or variants, isoforms or fragments thereof, or complements thereof may be used. In some embodiments, the target sequence is at least 15, 16, 17, 18, 19, 20, 22, 25, 50, 100, 200 continuous nucleotides or greater of the sequence of any of SEQ ID NOS: 139-173, or variants, isoforms or fragments thereof, or complements thereof. Methods to assay for the level of the RNA transcript, the level of the encoded polypeptide, or the activity of the polynucleotide or polypeptide are well known and discussed elsewhere herein.

Any region of the target sequence can be used to design the silencing element. For example, the silencing element may be designed to share sequence identity to 5′ untranslated region of the target sequence(s), 3′ untranslated region of the target sequence(s), exonic regions of the target sequence(s), and any combination thereof.

In certain embodiments, the silencing element shares sufficient identity, homology, or is complementary to at least about 15, 16, 17, 18, 19, 20, 22, 25 or 30 consecutive nucleotides from about nucleotides 1-50, 25-75, 75-125, 50-100, 125-175, 175-225, 100-150, 150-200, 200-250, 225-275, 275-325, 250-300, 325-375, 375-425, 300-350, 350-400, 425-475, 400-450, 475-525, 450-500, 525-575, 575-625, 550-600, 625-675, 675-725, 600-650, 625-675, 675-725, 650-700, 725-825, 825-875, 750-800, 875-925, 925-975, 850-900, 925-975, 975-1025, 950-1000, 1000-1050, 1025-1075, 1075-1125, 1050-1100, 1125-1175, 1100-1200, 1175-1225, 1225-1275, 1200-1300, 1325-1375, 1375-1425, 1300-1400, 1425-1475, 1475-1525, 1400-1500, 1525-1575, 1575-1625, 1625-1675, 1675-1725, 1725-1775, 1775-1825, 1825-1875, 1875-1925, 1925-1975, 1975-2025, 2025-2075, 2075-2125, 2125-2175, 2175-2225, 1500-1600, 1600-1700, 1700-1800, 1800-1900, 1900-2000 of the target sequence.

### Antisense Silencing Elements

As used herein, an “antisense silencing element” comprises a polynucleotide complementary to all or part of the target RNA transcript in the “antisense orientation”. Expression of the antisense silencing element reduces the level of the target polynucleotide or the polypeptide encoded thereby. The polynucleotide comprising the antisense silencing element may correspond to all or part of the complement of the sequence encoding the target polynucleotide, all or part of the complement of 5′ and/or 3′ untranslated region of the target polynucleotide, all or part of the complement of the coding sequence of the target polynucleotide, or all or part of the complement of both the coding sequence and the untranslated regions of the target polynucleotide.

Typically, an antisense silencing element has substantial sequence identity to the complement of the target sequence, typically greater than about 65% sequence identity, greater than about 70% sequence identity, greater than about 80% sequence identity, greater than about 85% sequence identity, about 90%, 91%, 92%, 93%, 94%, 95%, 96%, 97%, 98%, or 99% sequence identity. The antisense silencing element may be fully complementary (i.e., 100% identical) to the complement of the target sequence.

The antisense silencing element can be any length so long as it reduces the level of the target sequence. The antisense silencing element can be, for example, at least 15, 16, 17, 18, 19, 20, 22, 25, 30, 50, 100, 150, 200, 250, 300, 350, 400, 450, 500, 600, 700, 900, 1000, 1100, 1200, 1200, 1300, 1500, 1600, 1700, 1800 nucleotides of the complement of the target sequence of any of SEQ ID NOS: 36-104, or variants or fragments thereof, or complements thereof. In other embodiments, the antisense silencing element can be, for example, about 15-25, 19-35, 19-50, 25-100, 100-150, 150-200, 200-250, 250-300, 300-350, 350-400, 450-500, 500-550, 550-600, 600-650, 650-700, 700-750, 750-800, 800-850, 850-900, 900-950, 950-1000, 1000-1050, 1050-1100, 1100-1200, 1200-1300, 1300-1400, 1400-1500, 1500-1600, 1600-1700, 1700-1800 nucleotides or longer of the complement of the target polynucleotides of any of SEQ ID NOS: 36-104, or variants or fragments thereof, or complements thereof.

### Double Stranded RNA Silencing Element

A “double stranded RNA silencing element” or “dsRNA silencing element” comprises at least one transcript that is capable of forming a dsRNA either before or after ingestion or internalisation by a plant insect pest. Thus, a “dsRNA silencing element” includes a dsRNA, a transcript or polyribonucleotide capable of forming a dsRNA or more than one transcript or polyribonucleotide capable of forming a dsRNA.

“Double stranded RNA” or “dsRNA” refers to a polyribonucleotide structure formed either by a single self-complementary (or partially complementary) RNA molecule or a polyribonucleotide structure formed by the expression of at least two distinct RNA strands. The dsRNA molecule(s) employed in the disclosed methods and compositions mediate the reduction of expression of a target sequence, for example, by mediating RNA interference “RNAi” or gene silencing in a sequence-specific manner. In various embodiments, the dsRNA is capable of reducing the level of a target polynucleotide or the polypeptide encoded thereby in an insect plant pest.

The dsRNA can reduce the level of the target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). For example, see Verdel et al. (2004) Science 303:672-676; Pal-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237. Methods to assay for functional dsRNA that are capable of reducing the level of a target sequence are well known and disclosed elsewhere herein.

In certain embodiments, at least one strand of the duplex or double-stranded region of the dsRNA shares sufficient sequence identity or sequence complementarity to the target polynucleotide to allow the dsRNA to reduce the level of expression of the target sequence. In some embodiments, a dsRNA has substantial sequence identity to the target polynucleotide, typically greater than about 65% sequence identity, greater than about 70% sequence identity, greater than about 80% sequence identity, greater than about 85% sequence identity, about 90%, 91%, 92%, 93%, 94%, 95%, 96%, 97%, 98% or 99% sequence identity. The dsRNA may be fully complementary (i.e., 100% identical) to the target sequence, or the complement thereof.

The dsRNA can be any length so long as it reduces the level of the target sequence. The dsRNA can be, for example, at least 15, 16, 17, 18, 19, 20, 21, 22, 23, 24, 25, 50, 100, 200, 300, 400, 450 nucleotides of the sequence of any of SEQ ID NOS: 36-104, or variants or fragments thereof, or complements thereof. In some embodiments, dsRNA can be, for example, at least 15, 16, 17, 18, 19, 20, 21, 22, 23, 24, 25, 50, 100, 200, 300 nucleotides of any of SEQ ID NOS: 139-173, or variants or fragments thereof, or complements thereof.

In another embodiment, the dsRNA comprises a hairpin RNA. A hairpin RNA comprises an RNA molecule that is capable of folding back onto itself to form a double stranded structure. Multiple structures can be employed as hairpin elements. In certain embodiments, the dsRNA suppression element comprises an hairpin element which comprises in the following order, a first segment, a second segment, and a third segment, where the first and the third segment share sufficient complementarity to allow the transcribed RNA to form a double-stranded stem-loop structure.

The “second segment” of the hairpin comprises a “loop” or a “loop region”. These terms are used synonymously herein and are to be construed broadly to comprise any nucleotide sequence that confers enough flexibility to allow self-pairing to occur between complementary regions of a polynucleotide (i.e., segments 1 and 3 which form the stem of the hairpin). For example, in some embodiments, the loop region may be substantially single stranded and act as a spacer between the self-complementary regions of the hairpin stem-loop. In some embodiments, the loop region can comprise a random or nonsense nucleotide sequence and thus not share sequence identity to a target sequence. In other embodiments, the loop region comprises a sense or an antisense RNA sequence or fragment thereof that shares identity to a target sequence. In certain embodiments, the loop sequence can include an intron sequence, a sequence derived from an intron sequence, a sequence homologous to an intron sequence, or a modified intron sequence. The intron sequence can be one found in the same or a different species from which segments 1 and 3 are derived. In certain embodiments, the loop region can be optimized to be as short as possible while still providing enough intramolecular flexibility to allow the formation of the base-paired stem region. Accordingly, the loop sequence is generally less than 1000, 900, 800, 700, 600, 500, 400, 300, 200, 100, 50, 25, 20, 19, 18, 17, 16, 15, 10 nucleotides.

The “first” and the “third” segment of the hairpin RNA molecule comprise the base-paired stem of the hairpin structure. The first and the third segments are inverted repeats of one another and share sufficient complementarity to allow the formation of the base-paired stem region. In certain embodiments, the first and the third segments are fully complementary to one another. Alternatively, the first and the third segment may be partially complementary to each other so long as they are capable of hybridizing to one another to form a base-paired stem region. The amount of complementarity between the first and the third segment can be calculated as a percentage of the entire segment. Thus, the first and the third segment of the hairpin RNA generally share at least 50%, 60%, 70%, 80%, 85%, 90%, 91%, 92%, 93%, 94%, 95%, 96%, 97%, 98%, 99%, up to and including 100% complementarity.

The first and the third segment are at least about 1000, 500, 475, 450, 425, 400, 375, 350, 325, 300, 250, 225, 200, 175, 150, 125, 100, 75, 60, 50, 40, 30, 25, 22, 21, 20, 19, 18, 17, 16, 15 or 10 nucleotides in length. In certain embodiments, the length of the first and/or the third segment is about 10-100 nucleotides, about 10 to about 75 nucleotides, about 10 to about 50 nucleotides, about 10 to about 40 nucleotides, about 10 to about 35 nucleotides, about 10 to about 30 nucleotides, about 10 to about 25 nucleotides, about 10 to about 19 nucleotides, about 10 to about 20 nucleotides, about 19 to about 50 nucleotides, about 50 nucleotides to about 100 nucleotides, about 100 nucleotides to about 150 nucleotides, about 100 nucleotides to about 300 nucleotides, about 150 nucleotides to about 200 nucleotides, about 200 nucleotides to about 250 nucleotides, about 250 nucleotides to about 300 nucleotides, about 300 nucleotides to about 350 nucleotides, about 350 nucleotides to about 400 nucleotides, about 400 nucleotide to about 500 nucleotides, about 600 nucleotides, about 700 nucleotides, about 800 nucleotides, about 900 nucleotides, about 1000 nucleotides, about 1100 nucleotides, about 1200 nucleotides, about 1300 nucleotides, 1400 nucleotides, 1500 nucleotides, 1600 nucleotides, 1700 nucleotides, 1800 nucleotides, 1900 nucleotides, 2000 nucleotides or longer. In other embodiments, the length of the first and/or the third segment comprises at least 10-19 nucleotides, 10-20 nucleotides; 19-35 nucleotides; 20-35 nucleotides; 30-45 nucleotides; 40-50 nucleotides; 50-100 nucleotides; 100-300 nucleotides; about 500-700 nucleotides; about 700-900 nucleotides; about 900-1100 nucleotides; about 1300-1500 nucleotides; about 1500-1700 nucleotides; about 1700-1900 nucleotides; about 1900-2100 nucleotides; about 2100-2300 nucleotides; or about 2300-2500 nucleotides.

In some embodiments, the first and the third segment comprise at least 20 nucleotides having at least 85% complementary to the first segment. In still other embodiments, the first and the third segments which form the stem-loop structure of the hairpin comprise 3′ or 5′ overhang regions having unpaired nucleotide residues.

In other embodiments, the first and the third segments do not correspond to a target sequence. In these embodiments, the first and third segments flank a loop sequence that comprises a nucleotide sequence complementary to all or part of the target sequence. Thus, it is the loop region that determines the specificity of the RNA interference.

The sequence identity of the domains of the first, the second and/or the third segments complementary to a target polynucleotide need only be sufficient to reduce the level of the target sequence. See, for example, Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, A transient assay for the efficiency of hpRNA constructs to silence gene expression in vivo has been described by Panstruga et al. (2003) Mol. Biol. Rep. 30:135-140.

The amount of complementarity shared between the first, second, and/or third segment and the target sequence or the amount of complementarity shared between the first segment and the third segment (i.e., the stem of the hairpin structure) may vary. In some embodiments, 100% identity is required. In other embodiments, sequence variability may be tolerated as long a nucleotide region of the sequence (e.g., at least 22 nucleotides) is 100% identical to the target sequence.

Transcriptional gene silencing (TGS) may be accomplished through use of a hairpin suppression element where the inverted repeat of the hairpin shares sequence identity with the promoter region of a target sequence to be silenced. See, for example, Aufsatz et al. (2002) PNAS 99 (Suppl. 4): 16499-16506 and Mette et al. (2000) EMBO J 19(19): 5194-5201.

### Small RNA Silencing Element

A “small RNA” or “sRNA silencing element” can comprise both micro-RNA (miRNA) and short-interfering RNA (siRNA).

“miRNAs” are small single stranded non-coding RNAs comprising about 19 to about 24 ribonucleotides in length. miRNAs resemble the small interfering RNAs (siRNAs) of the RNA interference (RNAi) pathway, except miRNAs derive from regions of RNA transcripts that fold back on themselves to form short hairpins, whereas siRNAs derive from longer regions of double-stranded RNA.

Typically, the miRNA is transcribed from DNA sequence into primary miRNA (pri-miRNAs) and processed into precursor miRNA (pre-miRNAs) and mature miRNA.

For miRNA interference, the silencing element can be designed to express a dsRNA molecule that forms a hairpin structure or partially base-paired structure comprising a 19, 20, 21, 22, 23, 24 or 25 nucleotide sequence that is complementary to the target sequence. The miRNA can be synthetically made or transcribed as a longer RNA which is subsequently cleaved to produce the active miRNA.

When expressing a miRNA, the mature miRNA is present in a duplex in a precursor backbone structure, the two strands being referred to as the miRNA (the strand that will eventually base pair with the target sequence) and miRNA* (star sequence). miRNA precursors can be transgenically expressed.

The silencing element for miRNA interference may comprise a miRNA primary sequence. The miRNA primary sequence comprises a DNA sequence (genomic or cDNA) comprising the miRNA and star sequences separated by a loop as well as additional sequences flanking this region that are important for processing. When expressed, the structure of the primary miRNA is such as to allow for the formation of a hairpin RNA structure that can be processed into a mature miRNA.

In some embodiments, the miRNA precursor backbone comprises an heterologous miRNA and corresponding star sequence.

As used herein, a “star sequence” is the sequence within a miRNA precursor backbone that is complementary to the miRNA and forms a duplex with the miRNA to form the stem structure of a hairpin RNA.

In some embodiments, the star sequence can comprise less than 100% complementarity to the miRNA sequence. Alternatively, the star sequence can comprise at least 99%, 98%, 97%, 96%, 95%, 90%, 85%, 80% or lower sequence complementarity to the miRNA sequence as long as the star sequence has sufficient complementarity to the miRNA sequence to form a double stranded structure.

In still further embodiments, the star sequence comprises a sequence having 1, 2, 3, 4, 5 or more mismatches with the miRNA sequence and still has sufficient complementarity to form a double stranded structure with the miRNA sequence resulting in production of miRNA and reduction of the target sequence.

The miRNA precursor backbones can be from any plant or insect. In some embodiments, the miRNA precursor backbone is from a dicot. MicroRNA precursor backbones have been described previously (see, for example, Bally R, et al. (2020) Plant Biotechnol J 18(9): 1925-1932).

Thus, the primary miRNA can be altered to allow for efficient insertion of heterologous miRNA and star sequences within the miRNA precursor backbone. In such instances, the miRNA segment and the star segment of the miRNA precursor backbone are replaced with the heterologous miRNA and the heterologous star sequences, designed to target any sequence of interest, using a PCR technique and cloned into an expression construct. It is recognized that there could be alterations to the position at which the artificial miRNA and star sequences are inserted into the backbone. Methods for inserting the miRNA and star sequence into the miRNA precursor backbone are well known in the art.

When designing a miRNA sequence and star sequence, various design choices can be made. See, for example, Schwab R, et al. (2005) Dev Cell 8:517-27. In non-limiting embodiments, the miRNA sequences can have a “U” at 5′-end, a “C” or “G” at the 19th nucleotide position, and an “A” or “U” at the 10th nucleotide position. In other embodiments, the miRNA design is such that the miRNA have a high free delta-G as calculated using the ZipFold algorithm (Markham, N. R. & Zuker, M. (2005) Nucleic Acids Res. 33: W577-W581.) Optionally, a one base pair change can be added within 5′ portion of the miRNA so that the sequence differs from the target sequence by one nucleotide.

### Variants and Fragments

As used herein, “fragment” refers to a portion of the polynucleotide that retain the ability to reduce expression of the target sequence.

Fragments of a nucleotide sequence may range from at least about 10 nucleotides, about 15 nucleotides, about 16 nucleotides, about 17 nucleotides, about nucleotides 18, about 19 nucleotides, about 20 nucleotides, about 21 nucleotides, about 22 nucleotides, about 50 nucleotides, about 75 nucleotides, about 100 nucleotides, about 200 nucleotides, about 300 nucleotides, about 400 nucleotides, about 500 nucleotides, about 600 nucleotides, about 700 nucleotides and up to and including one nucleotide less than the full-length polynucleotide.

Alternatively, fragments of a nucleotide sequence may range from 1-50, 25-75, 75-125, 50-100, 125-175, 175-225, 100-150, 100-300, 150-200, 200-250, 225-275, 275-325, 250-300, 325-375, 375-425, 300-350, 350-400, 425-475, 400-450, 475-525, 450-500, 525-575, 575-625, 550-600, 625-675, 675-725, 600-650, 625-675, 675-725, 650-700, 725-825, 825-875, 750-800, 875-925, 925-975, 850-900, 925-975, 975-1025, 950-1000, 1000-1050, 1025-1075, 1075-1125, 1050-1100, 1125-1175, 1100-1200, 1175-1225, 1225-1275, 1200-1300, 1325-1375, 1375-1425, 1300-1400, 1425-1475, 1475-1525, 1400-1500, 1525-1575, 1575-1625, 1625-1675, 1675-1725, 1725-1775, 1775-1825, 1825-1875, 1875-1925, 1925-1975, 1975-2025, 2025-2075, 2075-2125, 2125-2175, 2175-2225, 1500-1600, 1600-1700, 1700-1800, 1800-1900, 1900-2000 of any one of SEQ ID NOS: 36-104, or variants, isoforms or complements thereof. In some embodiments the nucleotide sequence is from 1-50, 25-75, 75-125, 50-100, 125-175, 175-225, 100-150, 100-300, 150-200, 200-250, 225-275, 275-300, 250-300 of any one of SEQ ID NOS: 139-173, or variants, isoforms or complements thereof. In other embodiments, the nucleotide sequence is from 100-300 of any one of SEQ ID NOS: 139-173, or variants, isoforms or complements thereof. Methods to assay for the activity of a desired silencing element are well known and described elsewhere herein.

As used herein “variant” refers to a polynucleotide comprising one or more nucleotide deletions, insertions, substitutions that retains the ability to reduce expression of the target sequence. As used herein “variant” also refers to a polynucleotide comprising one or more modified sugars, nucleobases and/or internucleoside linkages.

As used herein, a “nucleoside” is a base-sugar combination and “nucleotides” are nucleosides that further include a phosphate group covalently linked to the sugar portion of the nucleoside.

As used herein, a nucleoside with a modified sugar residue is any nucleoside wherein the ribose sugar of the nucleoside has been substituted with a chemically modified sugar moiety. In the context of the present disclosure, the chemically modified sugar moieties include, but are not limited to, 2′-O-methoxyethyl, 2′-fluoro, 2′-dimethylaminooxyethoxy, 2′-dimethylaminoethoxyethoxy, 2′-guanidinium, 2′-O-guanidinium ethyl, 2′-carbamate, 2′-aminooxy, 2′-acetamido and locked nucleic acid. The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present disclosure, the term nucleobase also encompasses modified nucleobases. In this context “nucleobase” refers to both naturally occurring nucleobases such as adenine, guanine, cytosine, thymidine, uracil, xanthine and hypoxanthine, as well as non-naturally occurring variants.

In some embodiments, the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

In some embodiments, the modified internucleoside linkage increases the nuclease resistance of the polynucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing polynucleotides for in vivo use, and may serve to protect against nuclease cleavage at regions of DNA or RNA nucleosides in the polynucleotide of the invention, for example within the gap region of a gapmer polynucleotide, as well as in regions of modified nucleosides.

In some embodiments the internucleoside linkage comprises sulphur(S), such as a phosphorothioate internucleoside linkage. A phosphorothioate internucleoside linkage is particularly useful due to nuclease resistance, beneficial pharmacokinetics and ease of manufacture. In some embodiments at least 50% of the internucleoside linkages in the polynucleotide, or contiguous nucleotide sequence thereof, are phosphorothioate, such as at least 60%, such as at least 70%, such as at least 80 or such as at least 90% of the internucleoside linkages in the polynucleotide, or contiguous nucleotide sequence thereof, are phosphorothioate. In some embodiments all of the internucleoside linkages of the polynucleotide, or contiguous nucleotide sequence thereof, are phosphorothioate.

Other internucleoside linkages are known in the art and include, for example, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(RH)—O—, O—PO(OCH₃)—O—, —O—PO(NRH)—O—, —O—PO(OCH₂CH₂S—R)—O—, —O—PO(BH₃)—O—, —O—PO(NHRH)—O—, —O—P(O)₂—NRH—, —NRH—P(O)₂—O—, —NRH—CO—O—, —NRH—CO—NRH—; and —O—CO—O—, —O—CO—NRH—, —NRH—CO—CH₂—, —O—CH₂—CO—NRH—, —O—CH₂—CH₂—NRH—, —CO—NRH—CH₂—, —CH₂—NRHCO—, —O—CH₂—CH₂—S—, —S—CH₂—CH₂—O—, —S—CH₂—CH₂—S—, —CH₂—SO₂—CH₂—, —CH₂—CO—NRH—, —O—CH₂—CH₂—NRH—CO—, —CH₂—NCH₃—O—CH₂—, where RH is selected from hydrogen and C1-4-alkyl.

The term “modified polynucleotide” describes a polynucleotide comprising one or more sugar-modified nucleosides, modified nucleobases and/or modified internucleoside linkages.

The term “chimeric polynucleotide” can be used to describe a polynucleotide comprising bases that are a mix of different chemistries, or a gapmer, where some modifications are placed on the “wings” and not the central bases.

Generally, variants of a particular disclosed polynucleotide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

### Constructs

The use of the term “polynucleotide” is not intended to be limiting to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The disclosed polynucleotides also encompass all forms of sequences including, but not limited to, single-stranded forms, double-stranded forms, hairpins, stem-and-loop structures, and the like.

The polynucleotide encoding the silencing element or in certain embodiments employed in the disclosed methods and compositions can be provided in expression cassettes for expression in a plant or plant cell or host cell or organism.

It is recognized that multiple silencing elements including multiple identical silencing elements, multiple silencing elements targeting different regions of the target sequence, or multiple silencing elements targeting different target sequences can be used. In this embodiment, it is recognized that each silencing element may be encoded by a single or separate cassette, DNA construct, or vector. In some embodiments, the silencing element may be encoded by a plasmid, provided with an origin of replication. As discussed, any means of providing the silencing element is contemplated.

A plant or plant cell or host cell or organism can be transfected or transformed with a single cassette comprising DNA encoding one or more silencing elements or separate cassettes encoding a silencing element. Likewise, a plant or organism transformed with one component can be subsequently transformed with the second component. One or more DNA constructs encoding silencing elements can also be brought together by sexual crossing. That is, a first plant comprising one component is crossed with a second plant comprising the second component. Progeny plants from the cross will comprise both components.

Suitable host cells include the prokaryotes and the lower eukaryotes, such as fungi. Illustrative prokaryotes, both Gram-negative and Gram-positive, include Enterobacteriaceae, such as Escherichia, Erwinia, Shigella, Salmonella, and Proteus; Bacillaceae; Rhizobiceae, such as Rhizobium; Spirillaceae, such as Photobacterium, Zymomonas, Serratia, Aeromonas, Vibrio, Desulfovibrio, Spirillum; Lactobacilloae; Pseudomonadaceae, such as Pseudomonas and Acetobacter; Azotobacteraceae and Nitrobacteraceae. Among eukaryotes are fungi, such as Phycomycetes and Ascomycetes, which includes yeast, such as Saccharomyces and Schizosaccharomyces; and Basidiomycetes yeast, such as Rhodotorula, Aureobasidium, Sporobolomyces, and the like. Other eukaryotes include insect cells, for example, insect cells that can be infected by a virus such as a baculovirus.

Characteristics of particular interest in selecting a host cell may include ease of introducing the coding sequence into the host, availability of expression systems, efficiency of expression, stability in the host, and the presence of auxiliary genetic capabilities.

The silencing elements disclosed herein may be produced by introducing heterologous genes into a cellular host. For example, expression cassettes can be constructed which include the polynucleotide of interest operably linked with the transcriptional and translational regulatory signals for expression of the nucleotide constructs. The expression cassettes may also include a nucleotide sequence homologous with a sequence in the host cell, whereby integration will occur, and/or a replication system that is functional in the host, whereby integration or stable maintenance will occur. The expression cassettes may be transformed/transfected into a suitable host cell according to standard protocols of state of the art.

Transcriptional and translational regulatory signals include, but are not limited to, promoters, transcriptional initiation start sites, operators, activators, enhancers, other regulatory elements, ribosomal binding sites, an initiation codon, termination signals, and the like. See, for example, Sambrook et al. (2000); Molecular Cloning: A Laboratory Manual (3rd edition; Cold Spring Harbor Laboratory Press, Plainview, NY); Davis et al. (1980) Advanced Bacterial Genetics (Cold Spring Harbor Laboratory, Cold Spring Harbor, NY); and the references cited therein.

The expression cassette can include 5′ and 3′ regulatory sequences operably linked to the polynucleotide of the invention. “Operably linked” is intended to mean a functional linkage between two or more elements. For example, an operable linkage between a polynucleotide of the invention and a regulatory sequence (i.e., a promoter) is a functional link that allows for expression of the polynucleotide.

Operably linked elements may be contiguous or non-contiguous. The cassette may additionally contain at least one additional polynucleotide to be co-transformed into the plant or plant cell or host cell or organism. Alternatively, the additional polynucleotide(s) can be provided on multiple expression cassettes. Expression cassettes can be provided with a plurality of restriction sites and/or recombination sites for insertion of the polynucleotide to be under the transcriptional regulation of the regulatory regions. The expression cassette may additionally contain selectable marker genes.

The expression cassette can include in 5′-3′ direction of transcription, a transcriptional initiation region (i.e., a promoter), a polynucleotide encoding the silencing element employed in the methods and compositions of the invention, and a transcriptional termination region (i.e., termination region) functional in the plant, plant cell, or host cell or organism.

In other embodiments, the dsRNA is expressed from a suppression cassette. Such a cassette can comprise two convergent promoters that drive transcription of an operably linked silencing element.

“Convergent promoters” refers to promoters that are oriented on either terminus of the operably linked polynucleotide encoding the silencing element such that each promoter drives transcription of the silencing element in opposite directions, yielding two transcripts. In such embodiments, the convergent promoters allow for the transcription of the sense and anti-sense strand and thus allow for the formation of a dsRNA post-transcriptionally.

Such a cassette may also comprise two divergent promoters that drive transcription of one or more operably linked polynucleotides encoding the silencing elements.

“Divergent promoters” refers to promoters that are oriented in opposite directions of each other, driving transcription of the one or more polynucleotides encoding the silencing elements in opposite directions. In such embodiments, the divergent promoters allow for the transcription of the sense and antisense strands and allow for the formation of a dsRNA. In such embodiments, the divergent promoters also allow for the transcription of at least two separate hairpin RNAs.

In another embodiment, one cassette comprising two or more polynucleotides encoding the silencing elements under the control of two separate promoters in the same orientation is present in a construct. In another embodiment, two or more individual cassettes, each comprising at least one polynucleotide encoding the silencing element under the control of a promoter, are present in a construct in the same orientation.

The regulatory regions (i.e., promoters, transcriptional regulatory regions, and transcriptional termination regions) and/or the polynucleotides disclosed herein may be native/analogous to the host cell or to each other. Alternatively, the regulatory regions and/or the polynucleotide disclosed herein may be heterologous to the host cell or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

As used herein, a chimeric gene comprises a coding sequence operably linked to a transcription initiation region that is heterologous to the coding sequence.

The termination region may be native with the transcriptional initiation region, may be native with the operably linked polynucleotide encoding the silencing element, may be native with the host, or may be derived from another source (i.e., foreign or heterologous) to the promoter, the polynucleotide encoding the silencing element, the host, or any combination thereof. Termination regions include but are not limited to, prokaryotic terminators, like T7, T3, or SP6 terminators or the like. Other termination regions include those from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, re-substitutions, e.g., transitions and trans versions, may be involved.

A number of promoters can be used in the practice of the invention. The promoters can be selected based on the desired outcome. The nucleic acids can be combined with constitutive, tissue-preferred, inducible, or other promoters for expression in the host.

Such promoters include prokaryotic promoters, like T7, T3, or SP6 promoters or the like (Dunn and Studier (1983), J. Mol. Biol., 166, 477-535; Chakraborty, Salvo, Majumder, Maitra, (1977), Biol. Chem., 252, 6485-6493; Krieg and Melton (1987) Methods Enzymol., 155 397-415). Promoters and other expression signals may be selected to be compatible with the host cell for which the expression vector is designed. A suitable promoter for use is the inducible lacZ gene promoter, which is induced in the presence of Isopropylthiogalactosid (IPTG) (Hu and Davidson (1987), Cell, 48, 555-566). Other combinations are, for example, but not limited to any promoter which is controlled by the tetracycline repressor (Gossen and Bujard, (1992), Proc. Natl. Acad. Sci., USA, 89, 5547-5551), streptogramin-repressor (Fussenegger et al., (2000) Nature Biotechnol., 18, 1203-1208) or a inducible heat shock promoter.

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Depending on the desired outcome, it may be beneficial to express the gene from an inducible promoter. An inducible promoter, for instance, a pathogen-inducible promoter could also be employed. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156).

Tissue-preferred promoters can be utilized to target enhanced expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2): 157-168; Rinehart et al. (1996) Plant Physiol. 112(3): 1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6): 1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108. Such seed-preferred promoters include, but are not limited to, Ciml (cytokinin-induced message); cZ19BI (maize 19 kDa zein); and milps (myo-inositol-1-phosphate synthase) (see U.S. Pat. No. 6,225,529). Gamma-zein and Glob-1 are endosperm-specific promoters. For dicots, seed-specific promoters include, but are not limited to, bean Y-phaseolin, napin, Y-conglycinin, soybean lectin, cruciferin, and the like. A promoter that has “preferred” expression in a particular tissue is expressed in that tissue to a greater degree than in at least one other plant tissue. Some tissue-preferred promoters show expression almost exclusively in the particular tissue.

In an embodiment, the plant-expressed promoter is a vascular-specific promoter such as a phloem-specific promoter. A “vascular-specific” promoter, as used herein, is a promoter which is at least expressed in vascular cells, or a promoter which is preferentially expressed in vascular cells. Expression of a vascular-specific promoter need not be exclusively in vascular cells, expression in other cell types or tissues is possible. A “phloem-specific promoter” as used herein, is a plant-expressible promoter which is at least expressed in phloem cells, or a promoter which is preferentially expressed in phloem cells.

Expression of a phloem-specific promoter need not be exclusively in phloem cells, expression in other cell types or tissues, e.g., xylem tissue, is possible. In one embodiment of this invention, a phloem-specific promoter is a plant-expressible promoter at least expressed in phloem cells, wherein the expression in non-phloem cells is more limited (or absent) compared to the expression in phloem cells. Examples of suitable vascular-specific or phloem-specific promoters include but are not limited to the promoters selected from the group consisting of: the SCSV3, SCSV4, SCSV5, and SCSV7 promoters (Schunmann et al. (2003) Plant Functional Biology 30:453-60; the rolC gene promoter of Agrobacterium r n′zogewe\hat{ }Kiyokawa et al. (1994) Plant Physiology 104:801-02; Pandolfini et al. (2003) BioMedCentral (BMC) Biotechnology 3:7, (www.biomedcentral.com/1472-6750/3/7); Graham et al. (1997) Plant Mol. Biol. 33:729-35; Guivarc'h et al. (1996); Almon et al. (1997) Plant Physiol. 115:1599-607; the rolA gene promoter of Agrobacterium rhizogenes (Dehio et al. (1993) Plant Mol. Biol. 23:1199-210); the promoter of the Agrobacterium tumefaciens T-DNA gene 5 (Korber et al. (1991) EMBO J. 10:3983-91); the rice sucrose synthase RSsl gene promoter (Shi et al. (1994) /. Exp. Bot. 45:623-31); the CoYMV or Commelina yellow mottle badnavirus promoter (Medberry et al. (1992) Plant Cell 4:185-92; Zhou et al. (1998) Chin. J. Biotechnol. 14:9-16); the CFDV or coconut foliar decay virus promoter (Rohde et al. (1994) Plant Mol. Biol. 27:623-28; Hehn and Rhode (1998) /. Gen. Virol. 79:1495-99); the RTBV or rice tungro bacilliform virus promoter (Yin and Beachy (1995) Plant J. 7:969-80; Yin et al. (1997) Plant J. 12:1179-80); the pea glutamin synthase GS3A gene (Edwards et al. (1990) Proc. Natl. Acad. Sci. USA 87:3459-63; Brears et al. (1991) Plant J. 1:235-44); the inv CD111 and inv CD141 promoters of the potato invertase genes (Hedley et al. (2000) /. Exp. Botany 51:817-21); the promoter isolated from Arabidopsis shown to have phloem-specific expression in tobacco by Kertbundit et al. (1991) Proc. Natl. Acad. Sci. USA 88:5212-16); the VAHOX1 promoter region (Tornero et al. (1996) Plant J. 9:639-48); the pea cell wall invertase gene promoter (Zhang et al. (1996) Plant Physiol. 112:1111-17); the promoter of the endogenous cotton protein related to chitinase of US published patent application No. 20030106097, an acid invertase gene promoter from carrot (Ramloch-Lorenz et al. (1993) The Plant J. 4:545-54); the promoter of the sulfate transporter gene, Sultrl; 3 (Yoshimoto et al. (2003) Plant Physiol. 131:1511-17); a promoter of a sucrose synthase gene (Nolte and Koch (1993) Plant Physiol. 101:899-905); and the promoter of a tobacco sucrose transporter gene (Kuhn et al. (1997) Science 275-1298-1300).

Possible promoters also include the Black Cherry promoter for Prunasin Hydrolase (PH DL1.4 PRO) (U.S. Pat. No. 6,797,859), Thioredoxin H promoter from cucumber and rice (Fukuda A et al. (2005). Plant Cell Physiol. 46(11): 1779-86), Rice (RSsl) (Shi, T. Wang et al. (1994). /. Exp. Bot. 45(274): 623-631) and maize sucrose synthase-1 promoters (Yang., N-S. et al. (1990) PNAS 87:4144-4148), PP2 promoter from pumpkin Guo, H. et al. (2004) Transgenic Research 13:559-566), At SUC2 promoter (Truernit, E. et al. (1995) Planta 196(3):564-70., At SAM-1 (S-adenosylmethionine synthetase) (Mijnsbrugge K V. et al. (1996) Plant Cell. Physiol. 37(8): 1108-1115), and the Rice tungro bacilliform virus (RTBV) promoter (Bhattacharyya-Pakrasi et al. (1993) Plant J. 4(I): 71-79).

The expression cassette can also comprise a selectable marker gene for the selection of transformed cells. Selectable marker genes are utilized for the selection of transfected/transformed cells or tissues. Marker genes include genes encoding antibiotic resistance, such as those encoding ampicillin resistance, neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 76:215-28), cyan florescent protein (CYP) (Bolte et al. (2004) Cell Science 117:943-54 and Kato et al. (2002) Plant Physiol 729:913-42), and yellow florescent protein (PhiYFP™ from Evrogen, see, Bolte et al. (2004) Cell Science 777:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Bairn et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschmidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. The above list of selectable marker genes is not meant to be limiting. Any selectable marker gene can be used with the compositions and methods described herein.

### Compositions Comprising Silencing Elements

One or more silencing elements may be provided as an external composition such as a spray or powder to the plant, plant part, seed, a plant insect pest, or an area of cultivation. In another example, a plant is transformed with a DNA construct or expression cassette for expression of at least one silencing element. Either way, the silencing element, when internalised by an insect, can reduce the level of a target sequence and thereby control the pest.

In one embodiment, the composition is applied to a plant or seed (e.g., by spraying a field or area of cultivation) to protect the plant or seed from the pest. The composition may be applied simultaneously or in succession with other compounds (e.g., other insecticides). Methods of applying the composition include, but are not limited to, foliar application, seed coating, and soil application. The number of applications and the rate of application depend on the intensity of infestation by the pest.

The compositions may be applied by, for example, spraying, atomizing, dusting, scattering, coating or pouring, introducing into or on the soil, introducing into irrigation water, by seed treatment or general application or dusting at the time when the pest has begun to appear or before the appearance of pests as a protective measure.

A composition disclosed herein may further be formulated as bait. In this embodiment, the compositions comprise a food substance or an attractant which enhances the attractiveness of the composition to the pest.

A composition comprising the silencing element may be formulated in an agriculturally suitable and/or environmentally acceptable carrier. Such carriers may be any material that the plant or environment to be treated can tolerate. Furthermore, the carrier must be such that the composition remains effective at controlling a plant insect pest. Examples of such carriers include water, saline, Ringer's solution, dextrose or other sugar solutions, Hank's solution, and other aqueous physiologically balanced salt solutions, phosphate buffer, bicarbonate buffer and Tris buffer. In addition, the composition may include compounds that increase the half-life of a composition.

The components of the composition disclosed herein may be produced by introducing heterologous genes into a cellular host. Expression of the heterologous sequences results, directly or indirectly, in the intracellular production of a silencing element. These compositions may then be formulated in accordance with conventional techniques for application to the environment hosting a target pest, e.g., soil, water, and foliage of plants.

The compositions may also comprise one or more of: a penetrant, surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors, or other preparations that influence plant growth. One or more agrochemicals including, but not limited to, herbicides, insecticides, fungicides, bactericides, nematicides, molluscicides, acaracides, plant growth regulators, harvest aids, and fertilizers, can be combined with carriers, surfactants or adjuvants or other components to facilitate product handling and application for particular target pests. Suitable carriers and adjuvants can be solid or liquid and include for example, natural or regenerated mineral substances, solvents, dispersants, wetting agents, tackifiers, binders, or fertilizers.

Suitable surface-active agents include, but are not limited to, anionic compounds such as a carboxylate of, for example, a metal; carboxylate of a long chain fatty acid; an N-acylsarcosinate; mono- or di-esters of phosphoric acid with fatty alcohol ethoxylates or salts of such esters; fatty alcohol sulfates such as sodium dodecyl sulfate, sodium octadecyl sulfate, or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, for example, butyl-naphthalene sulfonate; salts of sulfonated naphthalene-formaldehyde condensates; salts of sulfonated phenol-formaldehyde condensates; more complex sulfonates such as the amide sulfonates, for example, the sulfonated condensation product of oleic acid and N-methyl taurine; or the dialkyl sulfosuccinates, for example, the sodium sulfonate or dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, for example, sorbitan fatty acid esters, condensation products of such esters with ethylene oxide, e.g., polyoxyethylene sorbitan fatty acid esters, block copolymers of ethylene oxide and propylene oxide, acetylenic glycols such as 2,4,7,9-tetraethyl-5-decyn-4,7-diol, or ethoxylated acetylenic glycols. Examples of a cationic surface-active agent include, for instance, an aliphatic mono-, di-, or polyamine such as an acetate, naphthenate or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials include, but are not limited to, inorganic minerals such as kaolin, phyllosilicates, carbonates, sulfates, phosphates, or botanical materials such as cork, powdered corncobs, peanut hulls, rice hulls, and walnut shells.

The compositions comprising a silencing element may be in a suitable form for direct application or as a concentrate of primary composition that requires dilution with a suitable quantity of water or other dilutant before application.

In some embodiments, virus-induced gene silencing (VIGS) is used to deliver one or more silencing elements to the insect plant pest itself. For example, an insect virus could be modified to contain a polynucleotide of the disclosure in its genome. Infection and replication of the virus would then lead to the production of silencing elements directly in the insect cells. A major advantage of this delivery method is that a very high efficiency can be achieved, even in otherwise recalcitrant cells. Relying on the virus's own infection processes, physiological and cellular barriers for the uptake of dsRNA from the environment are thus bypassed. Furthermore, viruses can be very host-specific, thereby providing another layer of species-specificity to this technology.

In other embodiments, a VIGS-like technology using various microbes, such as bacteria, yeast, or fungi that are engineered to serve as vectors to deliver one or more silencing elements to the insect plant pest itself.

A review of the use of bacteria and viruses for dsRNA delivery is provided in Joga et al. (Front. Physiol. (2016) 7:553) and Zotti et al. (Pest. Manag. Sci. (2018) 74:1239-1250). See, also, WO2000/063397 and WO20030522108.

### LDH Loaded Silencing Elements

Layered double hydroxides (LDHs) are mixed hydroxides of divalent and trivalent metals having an excess of positive charge that is balanced by interlayer anions. Common forms of LDH comprise Mg²⁺ and Al³⁺ (known as hydrotalcites) and Mg²⁺ and Fe³⁺ (known as pyroaurites) but LDHs containing other cations including Ni, Zn, Mn, Ca, Cr, and La are known. The amount of surface positive charge generated is dependent upon the mole ratio of the metal ions in the lattice structure, and the conditions of preparation as they affect crystal formation.

The LDH may have the general formula (1):

M^{II}₁₋ₓM^{III}ₓ(OH)₂Aⁿ⁻_{x/n}·yH₂O

where M^{II} and MIII are di- and tri-valent metal ions respectively and Aⁿ⁻ is the interlayer anion of valance n. The x value represents the proportion of bivalent metal to the total amount of metal ion present and y denotes variable, amounts of interlayer water. A limited portion of Aⁿ⁻ may be present on the LDH particle surface (for example, 5-40%, more especially 8-30% most especially 10-20%). This may explain why some dsRNA is adsorbed on the surface.

General formula (1) may also be written as formula (2);

M^{II}ₙM^{III}(OH)₂ₙ₊₁X·yH₂O

wherein X is one or more anions or negatively charged material to balance charge in the hydroxide layer. X is typically present in the interlayer space in the LDH material. A limited portion of X may be present on the LDH particle surface (for example, 5-40%, more especially 8-30% most especially 10-20%), This may explain why some dsRNA is adsorbed on the surface.

MII is suitably Mg, although other metal ions of valence 2+ may also be used. MIII is suitably Al. It will be appreciated that other metal ions of valence 3+ may also be used. Examples of other metal ions that may be used include:
M^{II}: Fe, Co, Ni, Cu, Zn, Mn, Pd, Ti, Cd and Ca
M^{III}: Co, Fe, Mn, Ga, Rh, Ru, Cr. V, In, Y, Gd, Ni and La.

These lists should not be considered to be limiting.

Exemplary anions in formulae (1) or (2) (i.e. Aⁿ⁻ or X) include, but are not limited to, (CO₃)²⁻; Cl⁻, (SO₄)²⁻; Cl⁻; OH; S²⁻ and [Sb(OH)₆].

The LDH may include a general layer of formula (3)

[M^{II}₁₋ₓM^{III}ₓ(OH)₂]X⁺

M^{II} and M^{III} and x are a defined above for formulae (1) and {2), and the positive charge x+ is balanced by anions (as may be described above for formulae (1) and (2)) which are intercalated between the layers.

The LDH may be of the hydrotalcite group, the quintinite group, the fougerite group, the woodwardite group, the cualstibite group, the glaueocerinite group, the wermlandite group, and the hydrocalumite group; especially of the hydrotalcite group; more especially hydrotalcite (Mg₆Al₂(OH)₁₆CO₃·4H₂O.

The hydrotalcite group is LDH of general formula (1), (2) or (3) in which M^{II}:M^{III} is 3:1 (especially in which M^{II} is Mg and M^{III} is Al) with a layer spacing of 6.8. to 8.8 \acute{Å}, especially of 7.3 to 8.3 \acute{Å}, more especially of 7.6 to 8.0 \acute{Å}, most especially about 7.8 \acute{Å}. A discussion on the hydrotalcite group, the quintinite group, the fougerite group, the woodwardite group, the cualstibite group, the glaucocerinite group, the wermlandite group, and the hydrocalumite group may be found in Mills et al., 2012 (Minerakogical Magazine 76:1289-1336). In another embodiment, the LDH is of general formula (1), (2) or (3) in which M^{II} is Mg and M^{III} is Al.

Exemplary LDH, and methods of making LDH, are described in Australian Patent No. 2005318862, the contents of which are incorporated herein by reference. Advantageously, in the method described in this patent the size of the LDH can be precisely controlled, and the hydrothermal treatment can disperse the LDH agglomerates into individual LDH particles.

The LDH particles may have a largest dimension within the range of up to 5 μm, more especially up to 1 μm, most especially up to 750 nm or up to 500 nm. In one embodiment, the LDH particles may have a largest dimension within the range 20-400 nm, more suitably 40-300 nm or 50-200 nm, even more suitably about 120 nm, with the thickness of the particles predominantly falling within the range of 5-40 nm, especially 1-20 nm. The particles may also exhibit a narrow particle size distribution, and the particles may show a particle size distribution of +20% around the average size. The LDH particles may have an aspect ratio that, falls within the range of from 5 to 10 (the ‘aspect ratio’ relates to the ratio of the largest dimension of the particle to its thickness or height). The LDH particles may combine together to form an average layer of 20-25 positively charged sheets.

The average particle size range for standard LDH is typically within about 10-60 micron, for example, about 10 micron, about 25 micron, or about 60 micron.

In one embodiment, the silencing element, for example, dsRNA is adsorbed onto the LDH has one or more of the dimensions, particle size distribution, or aspect ratio listed above for LDH particles. Scanning Electron Microscope images have confirmed that the morphology of the LDH particles is kept unchanged after loading dsRNA, as a result of the adsorption of dsRNA.

The dsRNA may be adsorbed onto the LDH by any suitable method, Advantageously, one method of adsorbing the dsRNA onto the LDH simply involves incubating the dsRNA with the LDH in an aqueous solution with shaking (for example, at from 100 to 300 rpm, especially at 200 rpm), and at a temperature front 20 to 50° C., especially from 25 to 45° C., or from 30 to 45° C., most especially about 37° C.

The dsRNA may also be adsorbed onto the LDH al any suitable loading ratio. Exemplary loading ratios (by mass) include from 2:1 to 1:20 dsRNA:LDH or from 1:1 to 1:10 dsRNA:LDH, more especially from 1:1 to 1:6 dsRNA:LDH or from 1:1 to 1:5 dsRNA:LDH, most especially from 1:1 to 1:4 dsRNA:LDH, from 1:1 to 1:2.5 dsRNA:LDH, from 1:2 to 1:5 dsRNA:LDH or from 1:3 to 1:4 dsRNA:LDH. The loading ratio may be 1:1, 1:1.5, 1:2, 1:2.5, 1:3, 1:3.5 or 1:4 dsRNA:LDH.

As used herein, the term “adsorbed” includes both circumstances in which dsRNA is adsorbed onto the surface of an LDH layer, as well as circumstances in which dsRNA is intercalated between LDH layers (which would inherently also involve some adsorption).

It may be advantageous when preparing the composition to use more dsRNA than can be adsorbed on the LDH. This is because if the dsRNA is completely adsorbed on the LDH, then dsRNA may not be immediately available on the plant; some of the LDH would need to break down, or some anion exchange must occur (for example via capture of CO₂ and conversion to HCO₃⁻CO₃²⁻ for anion exchange), before any dsRNA becomes available. Therefore, by using more dsRNA in the composition than can be adsorbed on the LDH, the composition can provide immediate protection to the plant after application. In one embodiment, from 50% to 95% of the dsRNA in the composition is adsorbed onto the LDH (allowing the remainder to be available as free dsRNA); especially from 60% to 90% of the dsRNA in the composition is adsorbed onto the LDH, most especially from 70% to 80% of the dsRNA in the composition is adsorbed onto the LDH. As a rough estimate, at a dsRNA:LDH mass ratio of 1:10, most dsRNA is adsorbed on the LDH surface. At a dsRNA:LDH mass ratio of 1:5, approximately 50% is adsorbed onto the LDH surface, approximately 30-40% is intercalated, and 10-20% is free in solution.

In one embodiment the dsRNA-LDH composition is in the form of a colloid or suspension, and it may include dsRNA-LDH particles at 10% w/w, especially up to 5% w/w or up to 2% w/w, even more especially about 1% w/w, most especially less than 1% w/w.

In another embodiment, dsRNA-LDH composition is in the form of a colloid or suspension, and it may include dsRNA-LDH particles at up to 100 mg/L, especially up to 50 mg/L, more especially up to 20 mg/L- or up to 10 mg/L mg/L; most especially less than 10 mg/L. In one embodiment, the concentration of dsRNA-LDH in a colloid or suspension is from 1-100 mg/L.

The composition may be formulated for administration to the plant, or to any part of the plant, in any suitable way. For example, the composition may be formulated for administration to the leaves, stem, roots, fruit vegetables, grains and/or pulses of the plant. In one embodiment, the composition is formulated for administration to the leaves of the plant and is especially sprayable onto the leaves of the plant. The composition may be administered to the plant as a metered dose. The composition may be formulated for administration to the plant, for example, by spraying, by brush or by another applicator.

The composition may also be administered to the plant at any suitable concentration, and advantageously the dsRNA in the composition may be effective at relatively low concentrations. In one embodiment, less than 100 μg of dsRNA per plant may be administered, especially less than 50 μg, more especially less than 40, 30, 20, 10 or 5 μg, most especially less than 1 μg or 0.5 μg of dsRNA per plant. When the composition is administered to the leaf of the plant, less than 100 μg of dsRNA per leaf may be administered to the plant, especially less than 50 μg, more especially less than 40, 30, 20, 10 or 5 μg, most especially less than 1 μg or 0.5 μg of dsRNA per leaf is administered to the plant.

### Penetrants

The term penetrant refers to materials which enhance the penetration of active compounds into plants, insects or both plants and insects, particularly materials which dissolve or penetrate the wax layer on the leaf surface of plants.

Suitable penetrants are substances that can improve the penetration of the silencing elements into the plant, plant pest or into both plant and plant pest. Examples of penetrants include mineral oils, vegetable oils, esterified vegetable oils, fatty acid esters and poly alkoxylate surfactants and sugar-based surfactants such as alkyl polyglycosides and glucamides. Preferably, the penetrant is selected from mineral oils, vegetable oils, esters of vegetable oils, fatty acid esters having 6-22 carbon atoms in the acid part (preferably 8 to 20 carbon atoms in the acid derived part) and 1-10 carbon atoms in the alcohol derived part, aromatic dicarboxylic acid esters having 1 to 8 carbon atoms in each alcohol derived part, alkanol alkoxylates, alkoxylated silicones and mixtures thereof. More specific examples of preferred penetrants include mineral oil, rapeseed oil (canola oil), sunflower oil, corn oil, linseed oil, oilseed rape (turnip rape) oil, olive oil, cottonseed oil, rapeseed oil methyl ester, rapeseed oil ethyl ester, ethylhexyl laurate, dibutyl succinate, dibutyl adipate, dibutyl phthalate, silicone-penetrants comprising a poly-C₂-C₄-alkyleneoxide modified polydimethylsiloxane (where the poly-C₂-C₄-alkyleneoxide may be of formula (AO) m); and alkanol alkoxylates of formula I:

′R—O-(AO)ₘ—R¹ (I)

wherein, R represents a linear or branched alkyl or alkenyl having from 4 to 20 carbon atoms, AO is C₂-C₄-alkylene oxide groups, i.e. ethylene oxide group (CH₂—CH2-O), Propylene oxide groups (CH(CH₃)—CH₂—O or CH₂—CH(CH₃)—O), butylene oxide groups (CH(C₂H₅)—CH₂—O, C(CH₃)₂—CH₂—O, CH₂—C(CH₃)₂—O or CH₂CH(C₂CH₅)—O, or a mixture of ethylene oxide and propylene oxide or butylene oxide groups, m Represents the number 1 to 30, in particular 2 to 20, and R¹ represents hydrogen or alkyl having 1 to 4 carbon atoms.

Particularly preferred examples of penetrants are selected from the group consisting of mineral oils, vegetable oils, esters of vegetable oils, polyalkoxylate surfactants and silicone surfactants. In one embodiment the penetrant is selected from paraffinic oil, crop oil, seed oil, methylated seed oil or ethylated seed oil that can dissolve or penetrate the wax layer on the leaf surface. Penetrants may also include those types of oils mixed with 0.5 to about 40% emulsifiers or surfactants to further enhance their utility and effectiveness.

Specific commercially available examples of preferred penetrants include alkoxylated polydimethyl siloxane non-ionic penetrant (active ingredient: 1020 g/L Polyether modified polysiloxane) available from Nufarm under the tradename PULSE® Penetrant (PULSE is a registered Trademark, or used under license by Nufarm Australia Limited), paraffin oil available under the trademark SUPERCHARGE® Elite spray tank adjuvant from Nufarm Australia Limited and methyl esters of canola oil fatty acids available under the trade marks BANJO® Spray Adjuvant and CANDO® adjuvant from Nufarm Australia Limited.

The content of penetrant in the compositions of the invention will depend on the nature of the composition and the required activity. Typically, the concentration is in the range of 0.01% w/w to 90% w/w. The composition may be in the form of a concentrate to be diluted prior to application to plants of may be a dilute composition formed for example by mixing components with water in a tank prior to application, for example, by spraying on to plants. In concentrate for the penetrant may for example comprise from 5% w/w to 95% w/w. in contrast dilute compositions for application may for example comprise 0.01% w/w to 5% w/w of the composition.

The weight ratio of penetrant:silencing element may vary widely depending on the type of composition and the dilution of the components in the composition. For example, in some embodiments the penetrant may be used as a carrier for the silencing dsRNA and/or dsRNA-LDH whereas in other composition the two components may be in distinct phases of a composition containing a plurality of phases such as an aqueous and oil phase, wherein the weight ratio of penetrant:silencing dsRNA is in the range of 1000:1 to 1:20.

Plants, Plant Parts, and Methods of Introducing Sequences into Plants

In one embodiment, the methods of the invention involve introducing a polynucleotide into a plant. “Introducing” is intended to mean presenting to the plant the polynucleotide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant.

Transformation protocols as well as protocols for introducing polynucleotide sequences into plants may vary depending on the type of plant or plant cell, e.g., dicot, targeted for transformation. Suitable methods of introducing polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lecl transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens).

In some embodiments, the polynucleotide may be introduced into plants by contacting plants with a virus or viral nucleic acids. Generally, such methods involve incorporating a nucleotide construct of the invention in a viral DNA or RNA molecule. Further, it is recognized that promoters may also encompass promoters utilized for transcription by viral RNA polymerases. Methods for introducing polynucleotides into plants involving viral DNA or RNA molecules are well known in the art. See, for example, U.S. Pat. Nos. 5,889,191, 5,889,190, 5,866,785, 5,589,367, 5,316,931, and Porta et al. (1996) Molecular Biotechnology 5:209-221.

Methods are well known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853. Briefly, the polynucleotides may be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants according to known methods. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the compositions and methods described herein provide transformed seeds (also referred to as “transgenic seed”) having a polynucleotide disclosed herein, for example, an expression cassette, stably incorporated into their genome.

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure.

The compositions and methods described herein may be used for transformation of any plant species, including, but not limited to, dicots. Examples of plant species of interest include, but are not limited to Row crops, Soybean, Cotton, Peanuts, Beans, Carrots, Tomato, Broccoli, Lettuce, Cucurbit crops such as Cucumber, Watermelon, Squash, Capsicum, Cabbage, Sweet potato, Eggplant, Ornamentals, Citrus, Chillies.

### Materials and Methods

### Whitefly Colony

The whitefly, B. tabaci (MEAM1, Biotype B) colonies were kindly provided by Prof. Gimmie Walter's lab at the University of Queensland, St. Lucia, Queensland. The whitefly colonies were obtained from a colony reared on eggplant (Solanum melongena) seedlings, and purity has been confirmed by Prof. Walter's lab using a molecular biotyping. The whiteflies were reared on Hibiscus rosa-sinensis in a growth chamber at 25±2° C., 50±5% relative humidity and 16 h light: 8 h dark photoperiod.

Total RNA Extraction and cDNA Synthesis from Whitefly

Total RNAs were extracted from different developmental stages of B. tabaci using TRIsure™ Reagent (Bioline, Australia) following the manufacturer's instructions. DNase I (NEB, Australia) treated 1 μg RNA was converted into cDNA using SensiFAST™ CDNA Synthesis Kit (Bioline, Australia) according to the manufacturer's protocol.

In-Vitro Synthesis and Fluorophore-Labeling of dsRNA

For RNAi experiments, selected fragments of B. tabaci genes were amplified from cDNA obtained from adult whiteflies using PCR. The template DNAs for in vitro transcription of dsRNAs were synthesized using gene-specific primers attached with T7 polymerase promoter sequence (TAATACGACTCACTATAGGG; SEQ ID NO:35) at the 5′ end (Table 1). PCR conditions were 95° C. for 1 min, then 35 cycles of 95° C. for 15 s, 55° C. to 65° C. for 15 s, 72° C. for 10 s, and extension step at 72° C. for 10 min. PCR products were purified using a Wizard SV Gel and PCR Clean-up System (Promega, USA). These purified DNAs were used to synthesize dsRNA using the Hi-Scribe T7 in vitro transcription kit (NEB, Australia) as per the manufacturer's instructions. Briefly, 1 μg of purified DNA was used as a template in a 20 μL in vitro transcription reaction. Each reaction was incubated for at least 4 h at 37° C., followed by 20 min of DNase treatment. The dsRNA was precipitated using 0.1× volume of 3M sodium acetate (pH 5.2) and 2.5× the volume of ice-cold 100% ethanol; incubated at −20° C. for 2 h followed by centrifugation at 4° C. for 20 min. The dsRNA pellet was then washed with 1 mL of 75% ethanol, air-dried and dissolved in nuclease-free water. The concentration of dsRNA was measured on spectrophotometer (Nano Drop 1000, Thermo Scientific US) and integrity was analysed by electrophoresis. dsRNA samples were stored at −20° C. prior to further use.

For fluorescent (Cy3)-labelling of the GFP and CMV 2b dsRNA, in vitro synthesis of dsRNA was performed as described above except the modified nucleotide in the reaction as follows: 10 mM ATP, 10 mM GTP, 10 mM UTP, 7.5 mM CTP and 2.5 mM Cy3-CTP (Perkin Elmer, Massachusetts, USA). The transcription reaction was incubated at 37° C. for 4 hours and then labelled dsRNA was purified using the RNeasy plus mini kit (Qiagen, Limberg, Netherlands) to avoid contamination of free Cy3-CTP in the final dsRNA solution. Confirmation of dsRNA labelling was confirmed by loading both Cy3-labeled and unlabelled dsRNAs onto 1% agarose gel.

### Selection of Target Genes and Off Target Effects

To design dsRNA constructs, a reference quality whitefly transcriptome was accessed via NCBI (https://ftp.ncbi.nlm.nih.gov/genomes/all/GCF/001/854/935/GCF_001854935.1_ASM1 85493v1/GCF_001854935.1_ASM185493v1_rna.fna.gz) and RNA-seq files were sourced from the NCBI Sequence read archive (Supplementary Table X). Read files in FASTQ format were adapter trimmed using Trim Galore (https://www.bioinformatics.babraham.ac.uk/projects/trim_galore/). Subsequently, transcript quantification was carried out using Sailfish (Sailfish enables alignment-free isoform quantification from RNA-seq reads using lightweight algorithms. Nature Biotechnology (doi:10.1038/nbt.2862)) and compiled using custom scripts.

For the generation of an off-target cohort k-mer set, the transcriptomes of Homo sapiens, Apis mellifera (REF), Apis florea (REF), Apis dorsata (REF), Apis cerana (REF) and Danaus plexippus (REF) were accessed via NCBI, with Jellyfish (https://academic.oup.com/bioinformatics/article/27/6/764/234905?login=true) used to count 14 nt k-mers in this dataset. Whitefly transcripts were scanned in an incrementing window of 400 nt to identify regions with the fewest intersecting k-mers with the off-target cohort. Within these regions Primer3 (https://www.ncbi.nlm.nih.gov/pmc/articles/PMC3424584/) via Primer3-py (https://libnano.github.io/primer3-py/) was used to design PCR primers (Table 1) that with the addition of 17 promoter sites could generate templates for in vitro dsRNA synthesis.

TABLE 1
Sequences of primers used for dsRNA synthesis

| | | Amplicon | SEQ |
|---|---|---|---|
| | | length | ID |
| Primer name | Sequence (5′~3′) | (bp) | NO: |
| CMV 2b | taatacgactcactatagggtatgtaattgaacgtaggtgcaat | 300 bp | 174 |
| dsRNA F | | | |
| CMV 2b | taatacgactcactatagggaaccaatctgtatogtcaaaatc | | 175 |
| dsRNA R | | | |
| GFP dsRNA_F | taatacgactcactatagggggcacaagctggagtacaa | 339 bp | 176 |
| GFP dsRNA_R | taatacgactcactatagggtctcgttggggtctttgctc | | 177 |
| BtGAP | taatacgactcactatagggtctacggagacgctgaataagc | 294 bp | 178 |
| dsRNA_F | | | |
| BIGAP | taatacgactcactatagggagacctttcaccatcgct | | 179 |
| dsRNA_R | | | |
| BtTreT | taatacgactcactatagggattccactcttggccatctg | 151 bp | 180 |
| dsRNA_F | | | |
| BtTreT | taatacgactcactatagggatctcctcgttgaccacctg | | 181 |
| dsRNA_R | | | |
| BtCOPB1 | taatacgactcactatagggcatcttggaaagtcgggctt | 252 bp | 182 |
| dsRNA_F | | | |
| BtCOPB1 | taatacgactcactatagggagttgatggggtcatcggtt | | 183 |
| dsRNA_R | | | |
| BtAIGluc | taatacgactcactatagggaatggcgagaccaagaattg | 312 bp | 184 |
| dsRNA_F | | | |
| BtAlGluc | taatacgactcactatagggcctggattgccttttggtaa | | 185 |
| dsRNA_R | | | |
| BtCHM4C | taatacgactcactatagggcacatggatgttgatcaagtgc | 258 bp | 186 |
| dsRNA_F | | | |
| BtCHM4C | taatacgactcactatagggcttgccacttgatttatctgcc | | 187 |
| dsRNA_R | | | |
| BtATP | taatacgactcactatagggtggagctctcagtagagcat | 163 bp | 188 |
| synthase | | | |
| dsRNA_F | | | |
| BtATP | taatacgactcactatagggttagtggtacttcctatagat | | 189 |
| synthase | | | |
| dsRNA_R | | | |
| BtN2B | taatacgactcactatagggcaacgtttgcagaactctgtg | 302 bp | 190 |
| dsRNA_F | | | |
| BtN2B | taatacgactcactatagggggctacggcattttcggatt | | 191 |
| dsRNA_R | | | |
| BtRieske | taatacgactcactatagggattgatcgttctttgctca | 188 bp | 192 |
| dsRNA_F | | | |
| BtRieske | taatacgactcactataggggacttgtcattgtcagaggt | | 193 |
| dsRNA_R | | | |
| BtAChE1 | taatacgactcactatagggccaagtttatcatcagacgga | 240 bp | 194 |
| dsRNA_F | | | |
| BtAChE1 | taatacgactcactatagggcccatgttgaaggagagaac | | 195 |
| dsRNA_R | | | |
| BtTreH | taatacgactcactatagggaaatctcccggttctggagt | 151 bp | 196 |
| dsRNA_F | | | |
| BtTreH | taatacgactcactatagggcagggtgaaatcctccttga | | 197 |
| dsRNA_R | | | |
| BtAqp1 | taatacgactcactatagggaaatcacgccaaaaacaggt | 200 bp | 198 |
| dsRNA_F | | | |
| BtAqp1 | taatacgactcactatagggagcaattgcgaatcctatcg | | 199 |
| dsRNA_R | | | |
| BtV-ATPase A | taatacgactcactatagggcagaactgaagtatgtgttgg | 212 bp | 200 |
| dsRNA_F | | | |
| BtV-ATPase A | taatacgactcactatagggcattctctcaaacgctggca | | 201 |
| dsRNA_R | | | |
| BtZFP | taatacgactcactatagggaacacagtgaccggtgcaa | 254 bp | 202 |
| dsRNA_F | | | |
| BtZFP | taatacgactcactataggggctggtcctgaagactatccat | | 203 |
| dsRNA_R | | | |
| BtGD | taatacgactcactatagggtgctcttggtggcttgactt | 270 bp | 204 |
| dsRNA_F | | | |
| BtGD | taatacgactcactatagggtacagcgtgtcttcgatccg | | 205 |
| dsRNA_R | | | |
| BtMYO | taatacgactcactatagggtgttcacccagactcaggtc | 298 bp | 206 |
| dsRNA_F | | | |
| BtMYO | taatacgactcactatagggtgcctcagagcttttccgtt | | 207 |
| dsRNA_R | | | |
| BtTryp_SPc | taatacgactcactataggggtcagaaaacgtcaagcgca | 279 bp | 208 |
| dsRNA_F | | | |
| BtTryp_SPc | taatacgactcactatagggatttgattgcgcgctgttga | | 209 |
| dsRNA_R | | | |
| BtDUOX | taatacgactcactatagggatggccagtgagagtggttg | 301 bp | 210 |
| dsRNA_F | | | |
| BtDUOX | taatacgactcactataggggcatggtatttcgaacgggc | | 211 |
| dsRNA_R | | | |
| BtPNLIPRP2 | taatacgactcactatagggtattctgagaccagtggcgc | 276 bp | 212 |
| dsRNA_F | | | |
| BtPNLIPRP2 | taatacgactcactataggggaccctgatccccaatccac | | 213 |
| dsRNA_R | | | |
| BtSUC | taatacgactcactatagggatgagcgaatggacatacat | 271 bp | 214 |
| dsRNA_F | | | |
| BtSUC | taatacgactcactataggggcaagctactgccggaattg | | 215 |
| dsRNA_R | | | |
| BtGTF | taatacgactcactatagggcagataattgtggcgcaccg | 302 bp | 216 |
| dsRNA_F | | | |
| BIGTF | taatacgactcactataggggggtaggaagcattgggtga | | 217 |
| dsRNA_R | | | |
| BtrBAT | taatacgactcactatagggatcatacgaacgtggaggcc | 294 bp | 218 |
| dsRNA_F | | | |
| BtrBAT | taatacgactcactatagggtgtccccaattttgaggcga | | 219 |
| dsRNA_R | | | |
| BLVDAC | taatacgactcactataggggaggtgagctggaacgccac | 299 bp | 220 |
| dsRNA_F | | | |
| BLVDAC | taatacgactcactatagggcactttcacgccgccggtca | | 221 |
| dsRNA_R | | | |
| BtCYP450 | taatacgactcactatagggatggtttacctgctctacg | 249 bp | 222 |
| dsRNA_F | | | |
| BtCYP450 | taatacgactcactatagggcttgactaggacgatcctgg | | 223 |
| dsRNA_R | | | |
| BtaATPase C | taatacgactcactataggggttggtagttgttccaaggg | 304 bp | 224 |
| dsRNA_F | | | |
| BtaATPase C | taatacgactcactataggggttaactttcaaccaccgaacc | | 225 |
| dsRNA_R | | | |
| BtaATPase E2 | taatacgactcactatagggccggatottcgtcaacaacc | 172 bp | 226 |
| dsRNA_F | | | |
| BtaATPase E2 | taatacgactcactatagggcggaaaactcagaaagtcggtc | | 227 |
| dsRNA_R | | | |
| BtaGABA4 | taatacgactcactatagggcgaaaatttccgctcgacacac | 152 bp | 228 |
| dsRNA_F | | | |
| BtaGABA4 | taatacgactcactataggggttgcgattaaactgtgttgagc | | 229 |
| dsRNA_R | | | |
| BtaGABA6 | taatacgactcactatagggggcagaaccactctaggtgt | 145 bp | 230 |
| dsRNA_F | | | |
| BtaGABA6 | taatacgactcactataggggtagagcgcaaaatacgaacgc | | 231 |
| dsRNA_R | | | |
| BtaCICP | taatacgactcactataggggtaatgaccagcttgataacg | 186 bp | 232 |
| dsRNA_F | | | |
| BtaCICP | taatacgactcactatagggtatcgccaggaaagcgagt | | 233 |
| dsRNA_R | | | |
| BtaGluR1 | taatacgactcactatagggctagcaggttggacatggc | 205 bp | 23 |
| dsRNA_F | | | |
| BtaGluR1 | taatacgactcactataggggagttctcctcgtagccgt | | 235 |
| dsRNA_R | | | |
| BtaCHM2b | taatacgactcactatagggtgagtctggagatgaagagg | 191 bp | 236 |
| dsRNA_F | | | |
| BtaCHM2b | taatacgactcactatagggagggcgtctttaataatttagc | | 237 |
| dsRNA_R | | | |
| BtaCMB7 | taatacgactcactataggggtcggtagatgaattcttgcaa | 227 bp | 238 |
| dsRNA_F | | | |
| BtaCMB7 | taatacgactcactatagggcgtagagatgatcaaattcgac | | 239 |
| dsRNA_R | | | |
| BtaSNF8 | taatacgactcactataggggaacaaacggcagtgctga | 216 bp | 240 |
| dsRNA_F | | | |
| BtaSNF8 | taatacgactcactatagggggagacaccatcactcacaca | | 241 |
| dsRNA_R | | | |
| BtaCHM6A | taatacgactcactatagggcattcagtgacactgacatgg | 192 bp | 242 |
| dsRNA_F | | | |
| BtaCHM6A | taatacgactcactataggggcttcttgctgtccttgact | | 243 |
| dsRNA_R | | | |
| BtaCHM3 | taatacgactcactatagggggatgaaaccatggactcag | 221 bp | 244 |
| dsRNA_F | | | |
| BtaCHM3 | taatacgactcactatagggcttgcaatcggttctgcat | | 245 |
| dsRNA_R | | | |
| BtaCOPG | taatacgactcactataggggttcggtttatgtactactca | 231 bp | 246 |
| dsRNA_F | | | |
| BtaCOPG | taatacgactcactataggggataaccttcatctgtgtcag | | 247 |
| dsRNA_R | | | |
| BtalAP | taatacgactcactatagggtggccaccttattcttcgc | 383 bp | 248 |
| dsRNA_F | | | |
| BtalAP | taatacgactcactatagggcgttgaaagcaaatggtgc | | 249 |
| dsRNA_R | | | |
| BtaBur | taatacgactcactatagggtagtttggtggctgtgtctc | 169 bp | 250 |
| dsRNA_F | | | |
| BtaBur | taatacgactcactatagggtaactggaacaacgaccgg | | 251 |
| dsRNA_R | | | |
| BtaSALP | taatacgactcactatagggttgtgtgaagaggtacacgg | 177 bp | 252 |
| dsRNA_F | | | |
| BtaSALP | taatacgactcactatagggagatgcctgtaatgcctgc | | 253 |
| dsRNA_R | | | |
| BtaTPS | taatacgactcactataggggttggatggccaggaattca | 200 bp | 254 |
| dsRNA_F | | | |
| BtaTPS | taatacgactcactataggggttctatccggcattgagtg | | 255 |
| dsRNA_R | | | |
| BtaFHBP | taatacgactcactatagggatgttcgagaacggctcctt | 282 bp | 256 |
| dsRNA_F | | | |
| BtaFHBP | taatacgactcactatagggatattgcgggctgaaaagcg | | 257 |
| dsRNA_R | | | |
| BtaChorion | taatacgactcactataggggaagcagttctcgccgaatc | 325 bp | 258 |
| dsRNA_F | | | |
| BtaChorion | taatacgactcactatagggcgcaggatacgtcgtcatca | | 259 |
| dsRNA_R | | | |
| BtaAmy | taatacgactcactatagggaccagctcgtttaaagcctca | 296 bp | 260 |
| dsRNA_F | | | |
| BtaAmy | taatacgactcactatagggggaaagcctggtcgaggtaa | | 261 |
| dsRNA_R | | | |
| BtaCytoc | taatacgactcactatagggggcgatggaaggacgatgat | 252 bp | 262 |
| dsRNA_F | | | |
| BtaCytoc | taatacgactcactatagggtcccatttcggtctatgatcca | | 263 |
| dsRNA_R | | | |
| BtaSurfeit | taatacgactcactatagggacttccagtggacacagcaa | 273 bp | 264 |
| dsRNA_F | | | |
| BtaSurfeit | taatacgactcactatagggctttgctgtcagccagaacg | | 265 |
| dsRNA_R | | | |
| BtaNAT15 | taatacgactcactatagggcagaaagcgtacgacgaagc | 270 bp | 266 |
| dsRNA_F | | | |
| BtaNAT15 | taatacgactcactatagggcgcatgagcatggggatact | | 267 |
| dsRNA_R | | | |
| BtaEH | taatacgactcactatagggcgttgatgtggtcccttgga | 256 bp | 268 |
| dsRNA_F | | | |
| BtaEH | taatacgactcactataggggttggcatttgtccacccaa | | 269 |
| dsRNA_R | | | |
| BtaZFP76 | taatacgactcactatagggggacggcaatgtcgaaatgg | 254 bp | 270 |
| dsRNA_F | | | |
| BtaZFP76 | taatacgactcactataggggcatcgctgtttccactagt | | 271 |
| dsRNA_R | | | |
| BtaCleavage | taatacgactcactatagggtcctgaacctgttcccttgc | 276 bp | 272 |
| dsRNA_F | | | |
| BtaCleavage | taatacgactcactatagggttagttgagcaacggcagga | | 273 |
| dsRNA_R | | | |
| BtaSyntax | taatacgactcactatagggctgcatattccagcaaccgc | 255 bp | 274 |
| dsRNA_F | | | |
| BtaSyntax | taatacgactcactataggggaaccggtgaaacgcaatca | | 275 |
| dsRNA_R | | | |
| Bta_Dyn | taatacgactcactatagggcgatgagagtgggaatcgca | 254 bp | 276 |
| dsRNA_F | | | |
| Bta_Dyn | taatacgactcactatagggtagcggatttggcctcagac | | 277 |
| dsRNA_R | | | |
| BtaProteosome | taatacgactcactatagggttgccgtcaaccatttgtcg | 291 bp | 278 |
| dsRNA_F | | | |
| BtaProteosome | taatacgactcactataggggcaacttccatctgaacggg | | 279 |
| dsRNA_R | | | |
| BtaCd6 | taatacgactcactataggggcgttgcttcccttgacaaa | 250 bp | 280 |
| dsRNA_F | | | |
| BtaCd6 | taatacgactcactatagggaaatcgacaccaccaagcct | | 281 |
| dsRNA_R | | | |
| BtaU5 | taatacgactcactataggggcactgaagcagagcaagtc | 293 bp | 282 |
| dsRNA_F | | | |
| BtaU5 | taatacgactcactatagggttgaggtcttccagcacgtc | | 283 |
| dsRNA_R | | | |
| BtaMediator | taatacgactcactatagggagagttgcgggacctgattt | 300 bp | 284 |
| dsRNA_F | | | |
| BtaMediator | taatacgactcactatagggggcccacctaagttttccca | | 285 |
| dsRNA_R | | | |
| BtaExp2 | taatacgactcactataggggctgcttgcactatcgaaaa | 266 bp | 286 |
| dsRNA_F | | | |
| BtaExp2 | taatacgactcactatagggggatttttggccacggcttt | | 287 |
| dsRNA_R | | | |
| BtacAMP | taatacgactcactatagggaactgtggggcattgatcgt | 290 bp | 288 |
| dsRNA_F | | | |
| BtacAMP | taatacgactcactatagggtaggcgaccaacttctacgg | | 289 |
| dsRNA_R | | | |
| BtaATPbind | taatacgactcactatagggccggccaaaaaccactcttc | 300 bp | 290 |
| dsRNA_F | | | |
| BtaATPbind | taatacgactcactatagggcatgtgacgccagaccaaac | | 291 |
| dsRNA_R | | | |
| BtaTre | taatacgactcactatagggggaacacgaggaccgctatt | 311 bp | 292 |
| dsRNA_F | | | |
| BtaTre | taatacgactcactatagggtgacctgtagattggcacgg | | 293 |
| dsRNA_R | | | |
| BtaZFP26 | taatacgactcactatagggagccagaagtgtggtaagcc | 332 bp | 294 |
| dsRNA_F | | | |
| BtaZFP26 | taatacgactcactatagggtctttgaacgtcttggggca | | 295 |
| dsRNA_R | | | |
| BtaKelch8 | taatacgactcactatagggccaatgcacgagccaagatc | 279 bp | 296 |
| dsRNA_F | | | |
| BtaKelch8 | taatacgactcactatagggactgtgccatcggtctgtta | | 297 |
| dsRNA_R | | | |
| BtaTre | taatacgactcactatagggatggtttgactgggacctgg | 251 bp | 298 |
| dsRNA_F | | | |
| BtaTre | taatacgactcactatagggttgcttgtaaaggtgcccac | | 299 |
| dsRNA_R | | | |
| BtaRRP42 | taatacgactcactatagggccaaaacaagcgtgctggaa | 274 bp | 300 |
| dsRNA_F | | | |
| BtaRRP42 | taatacgactcactatagggcctcttctgcagttggatcca | | 301 |
| dsRNA_R | | | |
| BtaKelch4 | taatacgactcactataggggctttggatgtttggtgggg | 262 bp | 302 |
| dsRNA_F | | | |
| BtaKelch4 | taatacgactcactataggggctgtagccgccgtagataa | | 303 |
| dsRNA_R | | | |
| BtaStill | taatacgactcactatagggtcagtggatgggatagggtgt | 265 bp | 304 |
| dsRNA_F | | | |
| BtaStill | taatacgactcactatagggaaaggcggcttgtaggtgaa | | 305 |
| dsRNA_R | | | |
| BtaKappaB | taatacgactcactatagggtgtgtccacgactgtccaag | 266 bp | 306 |
| dsRNA_F | | | |
| BtaKappaB | taatacgactcactatagggtgacccttgactgaccctct | | 307 |
| dsRNA_R | | | |
| BtaNFkappa | taatacgactcactatagggacacccgaatctaaccaccg | 263 bp | 308 |
| dsRNA_F | | | |
| BtaNFkappa | taatacgactcactatagggaaacccgcgttttcagtgtc | | 309 |
| dsRNA_R | | | |
| BtaATPaseAA | taatacgactcactatagggttgaaaggcgagcaactagc | 278 bp | 310 |
| A dsRNA_F | | | |
| BtaATPaseAA | taatacgactcactatagggacaaattctgcgtctccgga | | 311 |
| A dsRNA_R | | | |
| BtaExp4 | taatacgactcactataggggttcctggagctgaacactct | 263 bp | 312 |
| dsRNA_F | | | |
| BtaExp4 | taatacgactcactatagggaaattgacaacccagcgtgc | | 313 |
| dsRNA_R | | | |
| BtaSyntaxin | taatacgactcactatagggtggtcgctcacagtttacct | 311 bp | 314 |
| dsRNA_F | | | |
| BtaSyntaxin | taatacgactcactatagggtggagcttcaggcatttcgt | | 315 |
| dsRNA_R | | | |
| BtaNAT | taatacgactcactatagggcagaaagcgtacgacgaagc | 270 bp | 316 |
| dsRNA_F | | | |
| BtaNAT | taatacgactcactatagggcgcatgagcatggggatact | | 317 |
| dsRNA_R | | | |
| BtaSec31A | taatacgactcactataggggtgatgttgcggcttcttcc | 264 bp | 318 |
| dsRNA_F | | | |
| BtaSec31A | taatacgactcactatagggcttgcggctgataagttggc | | 319 |
| dsRNA_R | | | |
| BtaCysGly | taatacgactcactatagggtggaaaacccaaaatgcccg | 250 bp | 320 |
| dsRNA_F | | | |
| BtaCysGly | taatacgactcactatagggaagtgttcaccggtgtccat | | 321 |
| dsRNA_R | | | |
| BtaTPS3 | taatacgactcactatagggggagtgtggagtgagagcag | 261 bp | 322 |
| dsRNA_F | | | |
| BtaTPS3 | taatacgactcactataggggacaagtggtgtttgccgac | | 323 |
| dsRNA_R | | | |
| BtaGD | taatacgactcactataggggggagacacaaaccgggtat | 256 bp | 324 |
| dsRNA_F | | | |
| BtaGD | taatacgactcactatagggggtgaaccaattgagcctgc | | 325 |
| dsRNA_R | | | |
| BtaG6PD | taatacgactcactatagggcccgaagagcagatctaccg | 256 bp | 326 |
| dsRNA_F | | | |
| BtaG6PD | taatacgactcactatagggcaggtttctccatggccact | | 327 |
| dsRNA_R | | | |
| BtaFABP | taatacgactcactatagggcatccgccaaacaagacacc | 252 bp | 328 |
| dsRNA_F | | | |
| BtaFABP | taatacgactcactataggggcgtctcttcgtcgaactct | | 329 |
| dsRNA_R | | | |
| BtaJHIP | taatacgactcactatagggccttgcgtccaaagagaacg | 262 bp | 330 |
| dsRNA_F | | | |
| BtaJHIP | taatacgactcactatagggcaacaccgtcgattgatgca | | 331 |
| dsRNA_R | | | |
| BtaVit | taatacgactcactatagggaggaatgtgtgcactgagct | 264 bp | 332 |
| dsRNA_F | | | |
| BtaVit | taatacgactcactatagggtacatcgcgttgggcatgta | | 333 |
| dsRNA_R | | | |
| BtaDC | taatacgactcactatagggtgcacagaactcgaggttgt | 251 bp | 334 |
| dsRNA_F | | | |
| BtaDC | taatacgactcactataggggaggagtgggcttgtttgga | | 335 |
| dsRNA_R | | | |
| BtaRas | taatacgactcactatagggtgtggatactgcgggacaag | 288 bp | 336 |
| dsRNA_F | | | |
| BtaRas | taatacgactcactatagggtcgttttgctttgcagaggc | | 337 |
| dsRNA_R | | | |
| BtaJHEH1 | taatacgactcactatagggaccagacacagttgggacag | 282 bp | 338 |
| dsRNA_F | | | |
| BtaJHEH1 | taatacgactcactatagggtgtgcacagacggtaggaac | | 339 |
| dsRNA_R | | | |
| BtaJHEH2 | taatacgactcactatagggtactgctgcatagttggcct | 295 bp | 340 |
| dsRNA_F | | | |
| BtaJHEH2 | taatacgactcactatagggagccctaggtcattttcggg | | 341 |
| dsRNA_R | | | |
| BtaJHEBP | taatacgactcactatagggaggggttctatcgctcaacc | 320 bp | 342 |
| dsRNA_F | | | |
| BtaJHEBP | taatacgactcactatagggctggcgcatacaatctgcac | | 343 |
| dsRNA_R | | | |
| BtaAllato | taatacgactcactataggggggttgtttgtgacggtgc | 250 bp | 344 |
| dsRNA_F | | | |
| BtaAllato | taatacgactcactatagggtcacagagttatcatcgtccaca | | 345 |
| dsRNA_R | | | |
| BtaMtor | taatacgactcactatagggtcactgacactgatgccgat | 250 bp | 346 |
| dsRNA_F | | | |
| BtaMtor | taatacgactcactatagggcccaagccactgtgttcaac | | 347 |
| dsRNA_R | | | |
| BtaS6K | taatacgactcactatagggggctcctctcctgaagatgg | 272 bp | 348 |
| dsRNA_F | | | |
| BtaS6K | taatacgactcactatagggacccgaggcctgtacatttc | | 349 |
| dsRNA_R | | | |
| BtaAqp12 | taatacgactcactatagggatggcatcttagcgtacgca | 265 bp | 350 |
| dsRNA_F | | | |
| BtaAqp12 | taatacgactcactatagggacctgtaaatctgccgtgca | | 351 |
| dsRNA_R | | | |
| BtaT6PS | taatacgactcactatagggcaaaccgtttccagcttggg | 271 bp | 352 |
| dsRNA_F | | | |
| BtaT6PS | taatacgactcactatagggggacacaatacccgggtctc | | 353 |
| dsRNA_R | | | |
| BtarBAT | taatacgactcactatagggatcatacgaacgtggaggcc | 294 bp | 354 |
| dsRNA_F | | | |
| BtarBAT | taatacgactcactatagggtgtccccaattttgaggcga | | 355 |
| dsRNA_R | | | |
| BtaAGluc1 | taatacgactcactatagggtgctcggaaatcacgaccaa | 274 bp | 356 |
| dsRNA_F | | | |
| BtaAGluc1 | taatacgactcactataggggggttcgtcgaaaatcctgc | | 357 |
| dsRNA_R | | | |
| BtaAGluc2 | taatacgactcactatagggtccgcttctggatggacatg | 290 bp | 358 |
| dsRNA_F | | | |
| BtaAGluc2 | taatacgactcactataggggtagcgcatcttctcgtcc | | 359 |
| dsRNA_R | | | |
| BtaAGluc3 | taatacgactcactatagggcgataaaccgagagctgcct | 330 bp | 360 |
| dsRNA_F | | | |
| BtaAGluc3 | taatacgactcactatagggttggggcttttctcctgctt | | 36 |
| dsRNA_R | | | |
| BtaAGluc4 | taatacgactcactatagggagtgtgtactacgggaccga | 261 bp | 362 |
| dsRNA_F | | | |
| BtaAGluc4 | taatacgactcactatagggtctgttggaattttctgctc | | 363 |
| dsRNA_R | | | |
| BtaAGluc5 | taatacgactcactatagggactaccgcaatccgagagtg | 303 bp | 364 |
| dsRNA_F | | | |
| BtaAGluc5 | taatacgactcactatagggctgcatcgccgaggtaatct | | 365 |
| dsRNA_R | | | |
| BtaGTF | taatacgactcactatagggtgaagttggagcccttcacc | 292 bp | 366 |
| dsRNA_F | | | |
| BtaGTF | taatacgactcactatagggagccagtggtacaggaaacg | | 367 |
| dsRNA_R | | | |
| BtaNuc1 | taatacgactcactatagggtcatctctctcgccccgat | 274 bp | 368 |
| dsRNA_F | | | |
| BtaNuc1 | taatacgactcactatagggtctgcagcattctcggttgt | | 369 |
| dsRNA_R | | | |
| BtaNuc2 | taatacgactcactatagggcggttacagcttaaagccgc | 311 bp | 370 |
| dsRNA_F | | | |
| BtaNuc2 | taatacgactcactatagggtagtaaagctggcgtcacctc | | 371 |
| dsRNA_R | | | |
| BtaConstructI | taatacgactcactatagggcagaactgaagtatgtgttgg | 467 bp | 372 |
| dsRNA_F | | | |
| BtaConstructI | taatacgactcactataggggctggtcctgaagactatcca | | 373 |
| dsRNA_R | | | |
| BtaConstructII | taatacgactcactatagggccaagtttatcatcagacgga | 452 bp | 374 |
| dsRNA_F | | | |
| BtaConstructII | taatacgactcactatagggcattctctcaaacgctggcac | | 375 |
| dsRNA_R | | | |

### Artificial Diet Screening Bioassays

Diet feeding bioassays were performed to screen potential target genes in B. tabaci. Adult whiteflies (n=20) were collected from a lab colony using a vacuum aspirator and transferred to a petri-dish covered with a layer of parafilm M (Pechiney Plastic Packaging, IL, USA). An artificial diet comprised of 30% sucrose solution mixed with nuclease-free water or 200 ng/μl of target or GFP dsRNA was placed on the first layer of stretched parafilm. The second layer was stretched to cover the sucrose droplet without the formation of air bubble or spillage. Sucrose containing dsRNA solutions were fed to adult whiteflies for 6 consecutive days without replacing diet, and mortality was recorded. Sucrose and GFP dsRNA were used as controls in each feeding assay. The petri-dishes were kept in growth chamber at 25±2° C., 65±5% relative humidity and 16 h light: 8 h dark photoperiod. Experiments were repeated two to three times using 3 biological replicates with 75 adults per replicate; those with significant whitefly mortality were selected for further experiments and gene knockdown analysis.

Cotton pad soaking bioassays were performed to determine whether the B. tabaci specific target dsRNA has no undesirable effects on beneficial and non-target organisms, the Australian native stingless bee Tetragonula hockingsi. 30 adult bees were placed into a wooden box (10 cm*10 cm) and allowed to feed on sucrose (50%) solution mixed with nuclease-free water or Syntaxin dsRNA (200 ng/μl) soaked cotton pads. Cotton pads were changed every two days. The mortality of bees was calculated after 6 days. The wooden boxes were kept in growth chamber at 28±1° C., 50±5% relative humidity and 16 h light: 8 h dark photoperiod. Experiments were repeated two times using a 3 biological replicates with 30 bees per replicate.

Combination and Stacking of Target dsRNAs

Three target dsRNAs targeting vATPase A, AchE1 and ZFP were selected based on high mortality in screening bioassays and tested through mixing of RNAi approach. The target dsRNAs were mixed with sucrose 30% solution to a final concentration of 200 ng/μl (100 ng/μl of each dsRNA). Sucrose and GFP dsRNA were used as negative controls. Three biological replicates with 75 adult whiteflies per replicate were used in each assay.

Three stacked constructs (i) v-ATPase A and ZFP (467 bp) (ii) v-ATPase A and AchE1 (452 bp) and (iii) v-ATPase A, AchE1 and ZFP (707 bp) and mixture of two corresponding individual dsRNAs at a final concentration of 200 ng/μl were fed to whiteflies via artificial diet and mortality were observed up to six days.

dsRNA Loading on LDH

Before conducting insectary trials, a loading profile for each BioClay was performed to determine the ratio at which naked dsRNA is completely integrated with the LDH. Briefly, 500 ng of target in-vitro transcribed dsRNA were combined with varying amounts of LDH (loading from 1:1 to 1:6) and then incubated at room temperature for twenty minutes to allow loading of dsRNA onto the LDH. Complete dsRNA loading onto LDH was confirmed when the dsRNA-LDH remains visible in the well and unable to migrate through the 1% agarose gel. For diet feeding bioassays and insectary trials, appropriate loading ratios were selected with small amount of free dsRNA as it should be immediately available for triggering RNAi response in developmental stages of whitefly from the moment of foliar spray application.

### Confocal Microscopy—Plants

For confocal microscopy experiments, CMV2b-dsRNA was synthesized and labeled with Cy3 fluorophore (GE Healthcare, USA) using HiScribe T7 transcription kit (NEB, USA) as per the manufacturer's instructions. Cotton (Sicot-620) plants were grown in glasshouse at 25° C. (16 h: 8 h, light: day) for up to 4 weeks before being used for experiments.

To investigate whether incorporation of a penetrant to the dsRNA solution could enhance the uptake and internalization of dsRNA in plants, four-week-old cotton plants were treated with 5 μl droplets of 500 ng of CMV2b-dsRNA-Cy3, CMV2b-dsRNA-Cy3-banjo penetrant (0.05%), CMV2b-dsRNA-Cy3-pulse penetrant (0.01%), CMV2b-dsRNA-Cy3-supercharge elite penetrant (0.05%), and Cy3 only in replicates of two and incubated for 2 hours at room temperature in the dark.

To determine the translaminar movement of dsRNA within the cotton leaf, four-week-old cotton plants in replicates of two were applied with 5 μl droplets of Cy3 only, CMV2b-dsRNA-Cy3 (500 ng), CMV2b-dsRNA-Cy3-Banjo penetrant (0.05%), CMV2b-dsRNA-Cy3-Pulse penetrant (0.01%), and CMV2b-dsRNA-Cy3-Supercharge Elite penetrant (0.01%) onto the adaxial (upper) leaf surface. Plants were incubated for 24 hours at room temperature in the dark before being visualized under a confocal microscope from the adaxial and abaxial (lower) surfaces.

To detect the uptake of LDH released dsRNA in the cotton plants, 5 μl droplets of Cy3 only, CMV2b dsRNA-Cy3-Banjo penetrant (0.05%) (500 ng), and CMV2b-dsRNA-Cy3-LDH-Banjo penetrant (0.05%, 1:4) only were applied on cotton plants and incubated for 72 hours at room temperature in the dark.

After incubation, treated cotton leaves were washed by rinsing cotton leaf three times with Milli-Q water by vigorous pipetting. Confocal images were collected on a Leica Microsystems SP8 system (Leica, Wetzlar, Germany), excited with a tuneable white light laser and detected with HyD detectors. Leaf autofluorescence was characterized by varying the excitation laser, followed by detecting 20 nm bands for the full range of the HyD detector at different excitation laser settings. The leaf produced autofluorescence in the range of Cy3, and in the far-red region of the spectrum. Cy3 was collected Ex: 555 nm Em: 565-650 nm, leaf autofluorescence for structural information was collected Ex: 660 nm Em: 673-720 nm. Fluorescence lifetime imaging microscopy (FLIM: SP8 FALCON: a novel concept in fluorescence lifetime imaging enabling video-rate confocal FLIM. Alvarez, L. A. J et. al, Nature Methods, 2019. https://www.nature.com/articles/d42473-019-00261-x) was performed at 80 MHz to distinguish the autofluorescence from the leaf from the Cy3 fluorescence signal. Data was collected on leaf only, Cy3 only, and Cy3 dsRNA as microscopy controls. Data was collected to obtain a minimum of 100 photons per pixel to ensure reliable modelling of the fluorescence decay. Data was then analysed using the FLIMfit software tool developed at Imperial College London. The modelling parameters were used in a Matlab analysis package developed at the Institute for Molecular Bioscience, UQ to analyse the data and produce images of leaves with Cy3 labelled naked dsRNA or BioClay treatments void of autofluorescence. Z-stacks were used to verify that the fluorescence signal is inside the leaf and to measure the distance of Cy3 movement in the leaf.

### Confocal Microscopy—Whitefly

Artificial Diet-Mediated Uptake of dsRNA

To detect the uptake of dsRNA in adult whitefly through artificial diet, an artificial diet comprised 30% sucrose solution mixed with nuclease-free water or 100 ng/μl of Cy3-labeled GFP dsRNA was placed on the first layer of stretched parafilm. The second layer was stretched to cover the sucrose droplet without the formation of air bubble or spillage. The petri-dishes were wrapped in aluminium foil to avoid exposure to direct light and kept in a growth chamber at 25° C. (16 h: 8 h, light: day). After 24 hours, whiteflies were then harvested in liquid nitrogen (LN) and visualised under a confocal microscope.

Detached Leaf and Plant-Mediated Uptake of dsRNA

To determine the ability of adult whitefly to take up dsRNA from the detached cotton leaves, 500 μL of 50 ng/μl Cy3-CMV2b dsRNA or nuclease-free water (negative control) were added to a 1.5 ml micro-centrifuge tube and leaf petioles were then transferred into the tubes. After 24 hours, sixty adult whiteflies were transferred onto detached cotton leaves in 3 replicates (n=20 in 1 replicate) and allowed to feed for a further 48 hours. Whiteflies were then harvested and visualized under confocal microscope.

To investigate the uptake of dsRNA in whitefly from intact cotton plants, 50 μL of 100 ng/μl Cy3-labelled CMV2b dsRNA-Banjo penetrant (0.05%) or nuclease-free water (negative control) were pipetted on adaxial (upper) or abaxial (lower) surface of cotton leaves and allowed to dry for 24 hours. After 24 hours, sixty adult whiteflies (n=20 in 1 replicate) were transferred into the clip cages and allowed to feed on abaxial (lower) surface of three cotton plants for a further 48 hours. Whiteflies were then harvested in liquid nitrogen (LN) before being visualized under a confocal microscope.

Gene Expression Analysis by Quantitative Real-Time PCR (qRT-PCR)

For gene knockdown analysis, adult whiteflies (n=30) were collected in three biological replicates after four days of dsRNA ingestion, and RNA was extracted using TRIsure™ Reagent (Bioline, Australia). Quantitative real-time PCR (qRT-PCR) was performed using the SensiFAST SYBR No-ROX One-Step kit (Bioline, UK) as per manufacturer's instructions on a Rotor-Gene Q platform (QIAGEN, Netherland). The 20 μl qRT-PCR reaction was set up with 1× SensiFAST SYBR No-ROX one-step mix, 400 nM primers, reverse transcriptase, RiboSafe RNase Inhibitor, and 10 ng of total RNA. The thermocycler conditions were 45° C. for 10 min, 95° C. for 2 min, then 40 cycles at 95° C. for 5 s, 60° C. for 10 s, and 72° C. for 5 s. A melt-curve analysis was performed following end of each amplification run to confirm the specificity of the target PCR products. Reactions were run in two replicates and 50S ribosomal protein L6 (rpl6) (GeneBank accession number: XM_019058924.1) was used for transcript normalization. Relative expression levels were determined using the ΔΔCT method20. All the primers used in gene expression analysis were designed to avoid the target region used in the synthesis of dsRNA (Table 2).

TABLE 2
Sequences of primers used for qRT-PCR analysis

| | | Amplicon | SEQ |
|---|---|---|---|
| | | length | ID |
| Primer name | Sequence (5′~3′) | (bp) | NO: |
| RPL29_qRT_F | agaccaccgcaatggtatc | 142 bp | 376 |
| RPL29_qRT_R | ggacttcattctggcgtactg | | 377 |
| BtCOPB1 dsRNA_F | aaaagccccacctatcgttg | 150 bp | 378 |
| BtCOPB1 dsRNA_R | cactaaggttgccagctacag | | 379 |
| BtCHM4C dsRNA_F | gccaataccaacacagctgtat | 245 bp | 380 |
| BtCHM4C dsRNA_R | ggcctcagagatttccttagc | | 381 |
| BtAChE1 dsRNA_F | caactacatcttcggcgagc | 151 bp | 382 |
| BtAChE1 dsRNA_R | gtacacggatgcccaacttc | | 383 |
| BtTreH_qRT_F | cgcctactatcagctcaaacag | 155 bp | 384 |
| BtTreH_qRT_R | cgcacgtttcgacactgtaa | | 385 |
| BtAqp1_qRT_F | agccatctgtggagcaatcat | 196 bp | 386 |
| BtAqp1_qRT_R | ctttgatgtcggtccgctt | | 387 |
| BtV-ATPase A | gcccgcgagatcactcaact | 212 bp | 388 |
| dsRNA_F | | | |
| BtV-ATPase A | gtgagcgagtcgttgttgc | | 389 |
| dsRNA_R | | | |
| BtZFP dsRNA_F | gcatagacagcaagcacagc | 154 bp | 390 |
| BtZFP dsRNA_R | aggtccagagcctccaactc | | 391 |

### Plant-Based Insectary Trials

To evaluate the effects of target dsRNAs and dsRNA-LDH (BioClay) on whitefly eggs and nymphs, plant-based insectary trials were performed. Insectary trials consist of three cotton plants (with six leaves per treatment group) and includes water, LDH, and CMV2b-dsRNA (non-specific dsRNA targeting CMV strain 207) as controls. The workflow of insectary trial involved transferring 20 male and 20 female adult whiteflies (age irrelevant) collected in a glass vial using a vacuum aspirator into clip cages attached to the expanded leaves of 4-6-week-old cotton plants. The whitefly then deposited eggs (oviposition) for 24 hours. The adult whiteflies were then removed using an aspirator. Leaves sprayed with water, LDH only, CMV2b-dsRNA, target dsRNA and/or dsRNA-LDH (BioClay)-Ammonium sulfate (10 mM) post 1 and 10 days of eggs infestation and allowed to air dry. Plants were sprayed with approximately 1 ml of 0.5 μg/μl of dsRNA and/or 1.5 μg/μl of LDH unless specified. naked dsRNA and LDH-dsRNA (loading ratio 1:3) unless specified. The plants were then maintained in insect cages in a controlled insectary (at 25±1° C., photoperiod of 16:8 hr (Light:Dark), and RH 50%) for 17 days until whitefly egg hatching and 1st instar nymph emergence. On day 17, to enable counting under a microscope, cotton leaf petioles were detached and placed in glass vials containing water. Mortality ratios for eggs and nymphs were determined by calculating the abundance of unhatched eggs and dead nymphs relative to the total number of eggs and nymphs present.

To determine the impact of target dsRNAs and dsRNA-LDH (BioClay) on adult whiteflies, plant-based insectary trials were performed. Plant trials consisted of three to four cotton plants (with six-eight leaves per treatment group) and included water, LDH and CMV2b-dsRNA (non-specific) as control groups. Plants were sprayed with approximately 1 ml of 0.5 μg/μl of naked dsRNA and LDH-dsRNA (loading ratio 1:3) unless specified. Banjo (0.05%) penetrant was added to all treatment groups. First, cotton plants (4-6-week-old) were sprayed with controls, target dsRNA and/or dsRNA-LDH (BioClay)-Ammonium sulfate (10 mM) and allowed to dry for 24 hrs. Adult whiteflies (n=30) were collected in a glass vial using a vacuum pump and kept on ice for five minutes. to immobilize them. Whiteflies were then transferred into clip cages on expanded leaves of cotton plants. The plants were maintained in a climate controlled insectary (26±1° C., photoperiod of 16:8 (L:D) h, 50% relative humidity) for 8-11 days. Adult mortality was calculated by determining the abundance of dead adults relative to the total number of adults present.

### Statistical Analysis

The data are indicated as the mean+SE. Experimental data were analysed with Student's t-test or one-way analysis of variance in conjunction with Tukey's test to determine the significance of differences in mortality and gene knockdown using GraphPad prism 7.0 software. For all tests, P<0.05 was considered statistically significant.

### Results

### Identification of Novel Target Genes and Off-Target Analysis for RNAi-Mediated Control of Whitefly

For management of B. tabaci using RNAi approaches, novel target genes were identified by analysis of functional genomics data for insects and other closely related hemipteran species. In addition to the B. tabaci transcriptome expressions data, B. tabaci orthologues to other insect genes with promising RNAi phenotypes were searched with an expected value (e-value) threshold cut-off 1.0E-02 against the whitefly genome database (http://www.whiteflygenomics.org) and NCBI (https://blast.ncbi.nlm.nih.gov). Degradation of target transcripts in insects is dependent on the abundance and homology of dsRNA derived, Dicer-processed 20-22 nt siRNAs present in the cell's cytoplasm. As effective dsRNA and siRNA design strategies across insect orders are not well developed and likely variable given discrepancies in small RNA size and site cleavage preferences (https://www.mdpi.com/1999-4915/11/8/738, https://www.frontiersin.org/articles/10.3389/fphys.2018.01768/full), the present inventors focused on selecting dsRNA target sequences within target genes that minimised off-target homology. This ability to design dsRNA constructs that avoids unintended impacts on non-target organisms is a significant advantage for topically applied RNAi-based biopesticides relative to traditional chemical control measures. For an off-target cohort, humans (Homo sapiens) and key bioindicators honeybee (Apis mellifera, Apis florea, Apis dorsata, Apis cerana) and monarch butterfly (Danaus plexippus) were selected. As a proxy for sequence divergence, regions off approximately 400 bp within each whitefly transcript with the fewest contiguous 14 nt k-mers (sub-sequences) in common with the off-target cohort 14 nt k-mer set were identified, with in vitro dsRNA synthesis primer sites selected from within these regions. Using this approach, dsRNA constructs were designed for 96% of 15,662 transcripts.

As insects lack an RNA dependent RNA polymerase (RdRP)-based mechanism for amplification of the silencing signal (https://www.nature.com/articles/s41559-017-0403-4.pdf?proof=t), target transcripts that are too highly expressed may be difficult to sufficiently knock down due to the limited uptake inherit in a topical dsRNA application strategy. In contrast, target genes that are either not expressed or unevenly expressed during lifecycle stages overlapping with the dsRNA's window of protection are also unlikely to be efficacious targets. Accordingly, a transcript expression panel comprising multiple lifecycle stages including adult, nymph and egg was generated using publicly available RNA-seq datasets. This approach allowed for the selection of dsRNA constructs within a narrow band of expression (0.1-20 transcripts per million (TPM)) for subsequent use in high throughput screening assays.

In another approach, an off-target nucleotide homology threshold of 17 contiguous nucleotides was applied, and selected target genes were blasted against the transcriptomes of human, honeybees, monarch butterfly and other related species using the NCBI BLAST database and dsCheck (http://dscheck.rnai.jp/). Based on the BLAST outcomes, gene regions were identified as potential target sites and processed for PCR using T7 promoter attached gene-specific primers. The PCR products (145-467 bp) were then used to synthesize target dsRNAs for artificial diet (AD) and plant bioassays. Detailed information about selected ninety-four target genes and dsRNA sequences targeting these genes is given in Table 3.

### Screening of Potential Target Genes in Whitefly

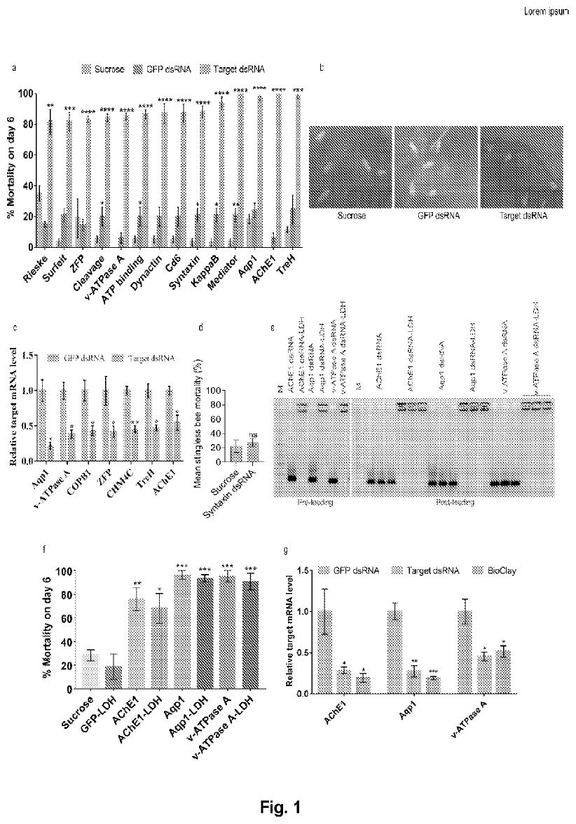

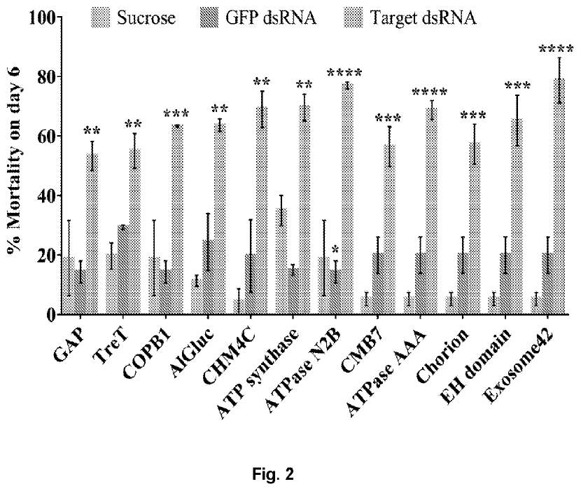

Genes involved in embryonic development, egg hatching, feeding, moulting, growth, and development of the whitefly were identified from functional genomics and RNAi-based studies. The functional annotations of the sixty-three genes and the dsRNA sequences to target these whitefly genes are found in Table 3. Artificial diet (sucrose 30%) bioassay was used to screen candidate target genes. Sucrose 30% and GFP dsRNA (non-specific dsRNA control) were included as controls to ascertain the effect of whitefly specific dsRNA. The percentage adult whitefly mortality resulting in Sucrose treatment and from each of the target dsRNA and GFP dsRNA when applied at concentration of 200 ng/μL is shown in FIG. 1a and FIG. 2. Twenty-six out of sixty-three dsRNA tested caused 53-100% mortality of whitefly as compared to negative controls (sucrose and GFP dsRNA) six days post-feeding (FIG. 1a, 1b and FIG. 2). Of the twenty-six dsRNAs, fourteen caused ≥80% mortality (FIG. 1a) and twelve resulted in ≤80% mortality (FIG. 2) when compared mortality in the controls. As a comparison only 5-35% mortality was observed in whiteflies fed on sucrose 30% and GFP dsRNA. To further test whether the selected candidate dsRNA could efficiently silence the target gene expression and link it to the observed mortality, whiteflies exposed to diet containing dsRNA were collected and the transcript abundance of some of the selected targets was determined by RT-qPCR. Quantification of mRNA levels of seven of these genes (Aqp1, v-ATPase A, COPB1, ZFP, CHM4C, TreH and AChE1) using RT-qPCR showed 45-78% knockdown in whiteflies fed on dsRNA targeting each of these genes (FIG. 1c). These data demonstrate that oral diet-based dsRNA feeding causes significant knockdown of target genes and mortality in whitefly and these promising candidate genes could serve as target sites for RNAi-mediated control of this pest.

To determine whether the B. tabaci specific syntaxin dsRNA affects the performance of the stingless bee, Tetragonula hockingsi, cotton pad soaking bioassays were performed. In this study, the mortality of bees was unaffected after 6 days of feeding on syntaxin dsRNA (FIG. 1d), suggesting the target dsRNA has no significant effects on bees.

Among the seven genes whose silencing (FIG. 1c) resulted in significant mortality of whitefly in screening bioassay, AChE1 (100%), V-ATPase A (85%) and ZFP (83%) were chosen for assessing the efficacy of these when delivered as BioClay (dsRNA-LDH). In the first instance this was tested in artificial diet bioassays. The selected target dsRNAs and GFP dsRNA as a non-specific-control were used to prepare dsRNA-LDH complex and checked for complete loading on the agarose 1% gel (FIG. 1e). The naked dsRNA or dsRNA-LDH complex were mixed with sucrose and fed to adult whiteflies for 6 days. The naked dsRNA targeting AChE1, Aqp1 and v-ATPase A showed 76%, 96%, and 91% mortality, whereas BioClay of these targets resulted in 68%, 93%, and 91% mortality, respectively, compared to sucrose and GFP dsRNA-LDH which showed 28% and 18% mortality, respectively (FIG. 1f). This demonstrated that the dsRNA loaded on LDH was bioavailable and caused similar mortality as naked dsRNA in AD assays. The gene knockdown efficiency of selected dsRNAs was determined using RT-qPCR. Oral diet feeding of naked dsRNA and BioClay targeting AChE1, Aqp1 and v-ATPase A reduced the target mRNA level by 72% and 81%, 74% and 81%, 55% and 49%, respectively, compared to GFP-LDH (FIG. 1g). These results indicate that dsRNA loaded on LDH or BioClay can effectively deliver dsRNA through artificial diet resulting in silencing of target gene expression and mortality in whitefly.

TABLE 3
List of target genes tested in artificial diet screening and insectary plant bioassays

| | | | | | | SEQ | Mortality >25% | Mortality >50% | |
|---|---|---|---|---|---|---|---|---|---|
| Target | | Functional | | Target | Good | ID | to <50% in | to <80% in | Mortality >80% |
| gene | Gene ID | annotation | Bioassay | stage | target? | NO: | bioassays | bioassays | in bioassays |
| BtaRpl29 | Bta02466 | 60S ribosomal | AD, Plant | N/A | N/A | 392 | | | |
| | | protein L29 | | | | | | | |
| CMV 2b | LC066508.1 | Cucumber | Plant | N/A | N/A | 393 | | | |
| | | mosaic virus | | | | | | | |
| | | genomic RNA | | | | | | | |
| GFP | MT612434.1 | Cloning vector | AD | N/A | N/A | 394 | | | |
| | | pMSCV-syn- | | | | | | | |
| | | Gephyrin. | | | | | | | |
| | | FingR-GFP | | | | | | | |
| BtGAP | Bta07742 | Hunchback | AD | Adult | ✓ | 139 | | ✓ | |
| BtTreT | Bta13849 | Solute carrier | AD | Adult | ✓ | 140 | | ✓ | |
| | | family 2, | | | | | | | |
| | | facilitated | | | | | | | |
| | | glucose | | | | | | | |
| | | transporter | | | | | | | |
| | | member 8 | | | | | | | |
| BtCOPB1 | Bta11961 | Coatomer | AD | Adult | ✓ | 141 | | ✓ | |
| | | subunit beta | | | | | | | |
| BtAlGluc | Bta07452 | Alpha- | AD | Adult | ✓ | 142 | | ✓ | |
| | | glucosidase | | | | | | | |
| BtCHM4C | Bta01993 | Charged | AD | Adult | ✓ | 143 | | ✓ | |
| | | multivesicular | | | | | | | |
| | | body | | | | | | | |
| | | protein | | | | | | | |
| | | 4C, putative | | | | | | | |
| BtATP | Bta09165 | ATP synthase | AD | Adult | ✓ | 144 | | ✓ | |
| synthase | | subunit f, | | | | | | | |
| | | mitochondrial | | | | | | | |
| BtN2B | Bta20011 | Putative | AD | Adult | ✓ | 145 | | ✓ | |
| | | ATPase N2B | | | | | | | |
| BtRieske | Bta11991 | Cytochrome | AD | Adult | ✓ | 146 | | | ✓ |
| | | b-c1 complex | | | | | | | |
| | | subunit | | | | | | | |
| | | Rieske, | | | | | | | |
| | | mitochondrial | | | | | | | |
| BtAChE1 | Bta05381 | Acetylcholin- | AD, Plant | Egg, | ✓ | 160 | | | ✓ |
| | | esterase | | Nymph, | | | | | |
| | | 1 | | Adult | | | | | |
| BtTreH | Bta00329 | Trehalase | AD, Plant | Adult | ✓ | 161 | | | ✓ |
| BtAqp1 | Bta01973 | Aquaporin | AD, Plant | Egg, | ✓ | 162 | | | ✓ |
| | | 1 | | Nymph, | | | | | |
| | | | | Adult | | | | | |
| Btv- | Bta13958 | V-type proton | AD, Plant | Egg, | ✓ | 163 | | | ✓ |
| ATPase A | | ATPase | | Nymph, | | | | | |
| | | subunit a | | Adult | | | | | |
| BtZFP | Bta11919 | Zinc | AD, Plant | Egg, | ✓ | 164 | | | ✓ |
| | | finger | | Nymph, | | | | | |
| | | protein | | Adult | | | | | |
| BtGD | Bta02405 | Glucose | Plant | Egg, | ✓ | 165 | ✓ | | |
| | | dehydrogenase | | Nymph, | | | | | |
| | | | | Adult | | | | | |
| BtMYO | Bta07326 | Myosin | Plant | Egg, | ✓ | 166 | ✓ | | |
| | | regulatory | | Nymph, | | | | | |
| | | light chain 2 | | Adult | | | | | |
| BtTryp_SPc | Bta03794 | Trypsin-like | Plant | Egg, | ✓ | 171 | ✓ | | |
| | | serine protease | | Nymph | | | | | |
| BtDUOX | Bta10996 | Dual | Plant | Egg, | ✓ | 167 | ✓ | | |
| | | oxidase | | Nymph, | | | | | |
| | | | | Adult | | | | | |
| BtPNLIPRP2 | Bta09442 | Pancreatic | Plant | Egg, | ✓ | 172 | ✓ | | |
| | | lipase-related | | Nymph | | | | | |
| | | protein 2 | | | | | | | |
| BtSUC | Bta14312 | Sucrase | Plant | Egg, | ✓ | 168 | ✓ | | |
| | | | | Nymph, | | | | | |
| | | | | Adult | | | | | |
| BtGTF | Bta13266 | Glycosyl | Plant | Egg, | [symbol] | | | | |
| | | transferase | | Nymph, | | | | | |
| | | | | Adult | | | | | |
| BtrBAT | Bta07377 | Neutral | Plant | Adult | [symbol] | | | | |
| | | and basic | | | | | | | |
| | | amino acid | | | | | | | |
| | | transport | | | | | | | |
| | | protein | | | | | | | |
| | | rBAT | | | | | | | |
| BtVDAC | Bta08782 | Voltage- | AD | Adult | [symbol] | | | | |
| | | dependent | | | | | | | |
| | | anion-selective | | | | | | | |
| | | channel | | | | | | | |
| BtCYP450 | Bta07221 | Cytochrome P450 | AD | Adult | [symbol] | | | | |
| BtaATPase C | Bta15459 | V-type proton | AD | Adult | [symbol] | | | | |
| | | ATPase | | | | | | | |
| | | subunit C | | | | | | | |
| BtaATPase E2 | Bta00642 | V-type proton | AD | Adult | [symbol] | | | | |
| | | ATPase | | | | | | | |
| | | subunit E 2 | | | | | | | |
| BtaGABA4 | Bta00850 | Gamma- | AD | Adult | [symbol] | | | | |
| | | aminobutyric | | | | | | | |
| | | acid receptor | | | | | | | |
| | | subunit | | | | | | | |
| | | alpha-4 | | | | | | | |
| BtaGABA | Bta14895 | Gamma- | AD | Adult | [symbol] | | | | |
| 6 | | aminobutyric | | | | | | | |
| | | acid receptor | | | | | | | |
| | | subunit | | | | | | | |
| | | alpha-6 | | | | | | | |
| BtaClCP | Bta10706 | Chloride | AD | Adult | [symbol] | | | | |
| | | channel protein | | | | | | | |
| BtaGluR1 | Bta10706 | Glutamate | AD | Adult | [symbol] | | | | |
| | | receptor-1 | | | | | | | |
| BtaCHM2b | Bta09410 | Charged | AD | Adult | [symbol] | | | | |
| | | multivesicular | | | | | | | |
| | | body protein | | | | | | | |
| | | 2b-B | | | | | | | |
| BtaCMB7 | Bta09904 | Charged | AD | Adult | ✓ | 147 | | ✓ | |
| | | multivesicular | | | | | | | |
| | | body protein 7 | | | | | | | |
| BtaSNF8 | Bta07537 | Vacuolar- | AD | Adult | [symbol] | | | | |
| | | sorting | | | | | | | |
| | | protein SNF8 | | | | | | | |
| BtaCHM6A | Bta00404 | Charged | AD | Adult | [symbol] | | | | |
| | | multivesicular | | | | | | | |
| | | body protein | | | | | | | |
| | | 6-A | | | | | | | |
| BtaCHM3 | Bta02800 | Charged | AD | Adult | [symbol] | | | | |
| | | multivesicular | | | | | | | |
| | | body protein 3 | | | | | | | |
| BtaCOPG | Bta07395 | Coatomer | AD | Adult | [symbol] | | | | |
| | | subunit gamma | | | | | | | |
| BtaIAP | Bta08342 | Baculoviral | AD | Adult | [symbol] | | | | |
| | | IAP repeat- | | | | | | | |
| | | containing | | | | | | | |
| | | protein 7 | | | | | | | |
| BtaBur | Bta08016 | Bursicon, | AD | Adult | [symbol] | | | | |
| | | putative | | | | | | | |
| BtaSALP | Bta13152 | Salivary | AD | Adult | [symbol] | | | | |
| | | protein 1310 | | | | | | | |
| BtaTPS | Bta15703 | Trehalose | AD | Adult | [symbol] | | | | |
| | | 6-phosphate | | | | | | | |
| | | synthase | | | | | | | |
| BtaFHBP | Bta02441 | Forkhead box | AD | Adult | [symbol] | | | | |
| | | protein B1 | | | | | | | |
| BtaChorion | Bta13877 | Chorion- | AD | Adult | ✓ | 148 | | ✓ | |
| | | specific | | | | | | | |
| | | transcription | | | | | | | |
| | | factor GCMa | | | | | | | |
| BtaAmy | Bta10119 | Amyloid beta A4 | AD, Plant | Adult | [symbol] | | | | |
| | | protein-binding | | | | | | | |
| | | family A | | | | | | | |
| | | member 1 | | | | | | | |
| BtaCytoc | Bta11782 | Cytochrome | AD | Adult | [symbol] | | | | |
| | | c-type | | | | | | | |
| | | heme lyase | | | | | | | |
| BtaSurfeit | Bta01046 | Surfeit locus | AD, Plant | Adult | ✓ | 149 | | | ✓ |
| | | protein 4 | | | | | | | |
| BtaNAT15 | Bta09847 | N-alpha-acetyl- | AD, Plant | Adult | [symbol] | | | | |
| | | transferase 15 | | | | | | | |
| BtaEH | Bta11007 | EH domain- | AD | Adult | ✓ | 150 | | ✓ | |
| | | containing | | | | | | | |
| | | protein 1 | | | | | | | |
| BtaZFP76 | Bta10540 | Zinc finger | AD | Adult | [symbol] | | | | |
| | | protein 76 | | | | | | | |
| BtaCleavage | Bta06507 | Cleavage | AD | Adult | ✓ | 151 | | | ✓ |
| | | stimulation | | | | | | | |
| | | factor | | | | | | | |
| | | subunit 3 | | | | | | | |
| BtaSyntax | Bta06944 | Syntaxin | AD, Plant | Adult | ✓ | 152 | | | ✓ |
| | | binding | | | | | | | |
| | | protein-1,2,3 | | | | | | | |
| BtaDyn | Bta02194 | Dynactin | AD, Plant | Egg, | ✓ | 169 | | | ✓ |
| | | subunit | | Nymph, | | | | | |
| | | | | Adult | | | | | |
| BtaProteosome | Bta07401 | 26S proteasome2 | AD | Adult | [symbol] | | | | |
| | | non-ATPase | | | | | | | |
| | | regulatory | | | | | | | |
| | | subunit 5 | | | | | | | |
| BtaCd6 | Bta11395 | Cdc6 | AD, Plant | Adult | ✓ | 153 | | | ✓ |
| BtaU5 | Bta03452 | U5 small nuclear | AD, Plant | Adult | [symbol] | | | | |
| | | ribonucleo | | | | | | | |
| | | protein | | | | | | | |
| | | 200 kDa helicase | | | | | | | |
| BtaMediator | Bta08552 | Mediator of RNA | AD, Plant | Adult | ✓ | 154 | | | ✓ |
| | | polymerase II | | | | | | | |
| | | transcription | | | | | | | |
| | | subunit 17 | | | | | | | |
| BtaExp2 | Bta06241 | Exportin-2 | AD | Adult | [symbol] | | | | |
| BtacAMP | Bta02506 | cAMP-dependent | AD | Adult | [symbol] | | | | |
| | | protein kinase | | | | | | | |
| | | type I | | | | | | | |
| | | regulatory | | | | | | | |
| | | subunit | | | | | | | |
| BtaATP | Bta10956 | ATP- | AD, Plant | Adult | ✓ | 155 | | | ✓ |
| binding | | binding | | | | | | | |
| | | cassette | | | | | | | |
| BtaTre | Bta03198 | Trehalase | AD | Adult | [symbol] | | | | |
| BtaZFP26 | Bta08770 | Zinc | AD | Adult | [symbol] | | | | |
| | | finger | | | | | | | |
| | | protein 26 | | | | | | | |
| BtaKelch8 | Bta09087 | Kelch-like | AD | Adult | [symbol] | | | | |
| | | protein 8 | | | | | | | |
| BtaTre | Bta03197 | Trehalase | AD | Adult | [symbol] | | | | |
| BtaRRP42 | Bta03733 | Exosome | AD, Plant | Adult | ✓ | 156 | | ✓ | |
| | | complex | | | | | | | |
| | | component RRP42 | | | | | | | |
| BtaKelch | Bta01226 | Kelch | AD | Adult | [symbol] | | | | |
| | | domain- | | | | | | | |
| | | containing | | | | | | | |
| | | protein 4 | | | | | | | |
| BtaStill | Bta00380 | Protein | AD, Plant | Adult | [symbol] | | | | |
| | | still life, | | | | | | | |
| | | isoform | | | | | | | |
| | | SIF type 1 | | | | | | | |
| BtaKappaB | Bta08258 | Nuclear factor | AD, Plant | Adult | ✓ | 157 | | | ✓ |
| | | kappa-B-binding- | | | | | | | |
| | | like protein | | | | | | | |
| BtaNFkappa | Bta02091 | NF-kappa- | AD | Adult | [symbol] | | | | |
| | | B-activating | | | | | | | |
| | | protein | | | | | | | |
| BtaATPaseAAA | Bta14342 | ATPase | AD, Plant | Adult | ✓ | 158 | | ✓ | |
| | | family | | | | | | | |
| | | AAA domain- | | | | | | | |
| | | containing | | | | | | | |
| | | protein 1-B | | | | | | | |
| BtaExp4 | Bta04332 | Exportin-4 | AD | Adult | [symbol] | | | | |
| BtaSyntaxin | Bta14843 | Syntaxin- | AD, Plant | Adult | [symbol] | | | | |
| | | binding | | | | | | | |
| | | protein 5 | | | | | | | |
| BtaNAT | Bta09847 | N-alpha- | AD | Adult | [symbol] | | | | |
| | | acetyl- | | | | | | | |
| | | transferase | | | | | | | |
| | | 15, NatA | | | | | | | |
| | | auxiliary | | | | | | | |
| | | subunit | | | | | | | |
| BtaSec31A | Bta06183 | Protein | AD | Adult | [symbol] | | | | |
| | | transport | | | | | | | |
| | | protein Sec31A | | | | | | | |
| BtaCysGly | Bta08216 | Cysteine and | Plant | Egg, | [symbol] | | | | |
| | | glycine-rich | | Nymph | | | | | |
| | | protein | | | | | | | |
| BtaTPS3 | Bta03375 | Transmembrane | Plant | Egg, | [symbol] | | | | |
| | | protease | | Nymph | | | | | |
| | | serine 3 | | | | | | | |
| BtaGD | Bta08909 | Glucose | Plant | Egg, | [symbol] | | | | |
| | | dehydrogenase | | Nymph | | | | | |
| BtaG6PD | Bta08749 | Glucose- | Plant | Adult | [symbol] | | | | |
| | | 6-phosphate 1- | | | | | | | |
| | | dehydrogenase | | | | | | | |
| BtaFABP | Bta15018 | Fatty acid | Plant | Egg, | [symbol] | | | | |
| | | binding | | Nymph | | | | | |
| | | protein | | | | | | | |
| BtaJHIP | Bta01142 | Juvenile | Plant | Egg, | [symbol] | | | | |
| | | hormone- | | Nymph | | | | | |
| | | inducible | | | | | | | |
| | | protein | | | | | | | |
| BtaVit | Bta14071 | Vitellogenin | Plant | Egg, | [symbol] | | | | |
| | | | | Nymph | | | | | |
| BtaDC | Bta07227 | Dopa- | Plant | Egg, | [symbol] | | | | |
| | | decarboxylase | | Nymph | | | | | |
| BtaRas | Bta12704 | Ras- | Plant | Egg, | [symbol] | | | | |
| | | related | | Nymph | | | | | |
| | | GTP | | | | | | | |
| | | binding | | | | | | | |
| | | protein | | | | | | | |
| BtaJHEH1 | Bta00739 | Juvenile | Plant | Egg, | [symbol] | | | | |
| | | hormone | | Nymph | | | | | |
| | | epoxide | | | | | | | |
| | | hydrolase 1 | | | | | | | |
| BtaJHEBP | Bta07266 | Juvenile | Plant | Egg, | [symbol] | | | | |
| | | hormone | | Nymph | | | | | |
| | | esterase | | | | | | | |
| | | binding | | | | | | | |
| | | protein | | | | | | | |
| BtaAllato | Bta02917 | Allatostatin | Plant | Egg, | [symbol] | | | | |
| | | double | | Nymph | | | | | |
| | | C, isoform C | | | | | | | |
| BtaMtor | Bta15399 | Serine/threonine- | Plant | Egg, | [symbol] | | | | |
| | | protein kinase | | Nymph | | | | | |
| | | Mtor | | | | | | | |
| BtaS6K | Bta04220 | S6 kinase- | Plant | Egg, | [symbol] | | | | |
| | | like protein | | Nymph | | | | | |
| BtaAqp12 | Bta14320 | Aquaporin-12 | Plant | Adult | [symbol] | | | | |
| BtaT6PS | Bta08458 | Trehalose | Plant | Adult | [symbol] | | | | |
| | | 6- | | | | | | | |
| | | phosphate | | | | | | | |
| | | synthase | | | | | | | |
| BtarBAT | Bta07377 | Neutral | Plant | Adult | ✓ | 159 | ✓ | | |
| | | and basic | | | | | | | |
| | | amino acid | | | | | | | |
| | | rBAT | | | | | | | |
| BtaAGluc1 | Bta05397 | Alpha | Plant | Adult | [symbol] | | | | |
| | | glucosidase | | | | | | | |
| BtaAGluc2 | Bta03991 | Alpha | Plant | Adult | [symbol] | | | | |
| | | glucosidase | | | | | | | |
| BtaAGluc3 | Bta03992 | Alpha | Plant | Adult | [symbol] | | | | |
| | | Glucosidase | | | | | | | |
| BtaAGluc4 | Bta08428 | Alpha | Plant | Adult | [symbol] | | | | |
| | | glucosidase | | | | | | | |
| BtaAGluc5 | Bta04421 | Alpha | Plant | Adult | [symbol] | | | | |
| | | Glucosidase | | | | | | | |
| BtaGTF | Bta07245 | Glycosyl | Plant | Adult | ✓ | 173 | ✓ | | |
| | | transferase | | | | | | | |
| BtaNucI | Bta11788 | Deoxyribo | Plant | Egg, | [symbol] | | | | |
| | | nuclease II | | Nymph, | | | | | |
| | | | | Adult | | | | | |
| BtaNucII | Bta06243 | Deoxyribo- | Plant | Egg, | ✓ | 170 | ✓ | | |
| | | nuclease I | | Nymph, | | | | | |
| | | | | Adult | | | | | |
| Construct- | Bta13958 | (v-ATPase-ZFP) | | Egg, | ✓ | 395 | | | ✓ |
| I | Bta11919 | Stacked | | Nymph, | | | | | |
| | | construct | | Adult | | | | | |
| Construct- | Bta05381 | (AChE1-Btv- | AD | Adult | [symbol] | | | | |
| II | Bta13958 | ATPase A) | | | | | | | |
| | | Stacked | | | | | | | |
| | | construct | | | | | | | |
| Construct- | Bta13958 | V-ATPase A- | AD | Adult | [symbol] | | | | |
| III | Bta05381 | AChE1-ZFP | | | | | | | |
| | Bta11919 | | | | | | | | |

Combination and Stacking of dsRNAs to Improve RNAi Effects

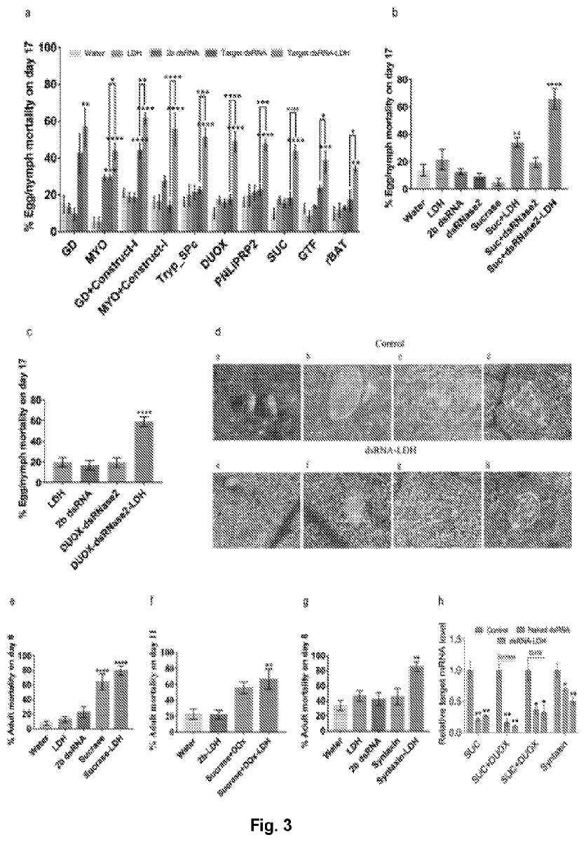

To investigate whether the RNAi-mediated mortality can be enhanced by targeting more than one gene in adult whitefly, combination of two effective dsRNA targets were tested in artificial diet bioassays. Two approaches were used to assess combinatorial RNAi effects including (i) mixing the two target dsRNAs or (ii) stacking two dsRNAs into single expression construct. For the approach of expressing the dsRNAs as single targets and mixing thereafter, three dsRNAs targeting v-ATPase A, ZFP, and AchE1 were selected based on their gene silencing and mortality in artificial diet bioassays. The selected single dsRNA (100 ng/μl) or mixture of two dsRNAs (100 ng/μl of each dsRNA) were fed to whitefly adults. All three combinations of dsRNAs AChE1 and v-ATPase A (FIG. 3a), AChE1 and ZFP (FIG. 3b), v-ATPase A and ZFP (FIG. 3c) showed 92%, 74% and 94% mortality, respectively as compared to single dsRNA in resulted showing 46% and 63%, 46% and 55%, and 55% and 46% mortality, respectively. Comparatively, sucrose and GFF dsRNA resulted in only 7-31% mortality. This significantly higher whitefly mortality in mixed dsRNAs groups compared to feeding the whiteflies with the individual dsRNAs demonstrates that the combinations of dsRNAs is more effective (FIGS. 3a, 3b, and 3c). This increase in RNAi-mediated mortality suggests that it is feasible to target two genes simultaneously in the whitefly by combination of dsRNAs. The next question that was addressed whether increased RNA-mediated mortality was also reflected in decrease in gene expression of both the target genes. Whiteflies were fed on single or mixture of dsRNAs for 4 days and surviving insects were collected for gene expression analysis by RT-qPCR. The mixed dsRNAs AChE1 and v-ATPase A (FIGS. 3d and 3e), AChE1 and ZFP (FIGS. 3f and 3g), v-ATPase A and ZFP (FIGS. 3h and 3i) reduced mRNA expression of both target genes 78% and 62%, 52% and 59%, and 63% and 72%, respectively, compared to whiteflies fed on non-specific GFP dsRNA. The single dsRNA targeting AChE1 and v-ATPase A (FIGS. 3d and 3e), AChE1 and ZFP (FIGS. 3f and 3g), v-ATPase A and ZFP (FIGS. 3h and 3i) caused 61% and 63%, 52% and 68%, and 74% and 61% reduction in target mRNA levels in whitefly. These results clearly demonstrate that mixing of dsRNAs can significantly improve RNAi-mediated control of B. tabaci via diet feeding.

Studies were also conducted to investigate whether stacking of dsRNAs targeting v-ATPase A, AchE1 and ZFP into a single expression vector will give the same result to what was observed by mixing the dsRNAs post expression as single targets. Two stacked dsRNAs namely construct-I (v-ATPase A and ZFP) and Construct-II (v-ATPase A and AchE1) expressed as single construct and mixture of the same dsRNAs were fed to adult whiteflies via artificial diet and mortality were recorded up to six days. Whiteflies fed on stacked construct-I and stacked construct-II dsRNA showed 93% and 72% mortality, respectively, whereas insects exposed to mixture of the same dsRNAs resulted in 56% and 62% mortality (FIGS. 3j and 3k), respectively. In comparison, sucrose and GFP dsRNA showed only 4-8% mortality. Interestingly, out of the two selected stacked dsRNAs, construct-I only showed significantly higher whitefly mortality than corresponding mixture of dsRNAs. Although these results indicated that the stacking of dsRNAs can improve whitefly mortality, the effect of dsRNAs in this stacked dsRNA treatment could be variable than mixture of same dsRNAs due to differences in siRNA pool generation by dicer. To test whether the stacked construct-I resulted in mortality due to the silencing of both target genes, whiteflies were fed on mixed or stacked dsRNAs (construct-I) targeting v-ATPase A and ZFP for 4 days. Surviving whiteflies were collected and RT-qPCR was performed. Whiteflies fed on stacked dsRNAs showed 57% and 45% reduction in v-ATPase A and ZFP mRNA levels, respectively, whereas mixed dsRNA resulted in only 23% and 24% decrease in target mRNA expression, respectively as compared to GFP dsRNA (FIGS. 3l and 3m). These results suggest that stacking of dsRNAs can significantly increase RNAi-mediated mortality by suppressing both target genes simultaneously.

### Foliar Spray of BioClay for Whitefly Control

The life cycle of whitefly is composed of six-stages, which limits the potential for a single pesticide application to eradicate the various developmental stages, including egg, nymph and adult. To address this issue, the present inventors investigated for the first time whether foliar spray of naked dsRNA and dsRNA-LDH (BioClay) could be used to provide RNAi-mediated control against developmental stages of whitefly.

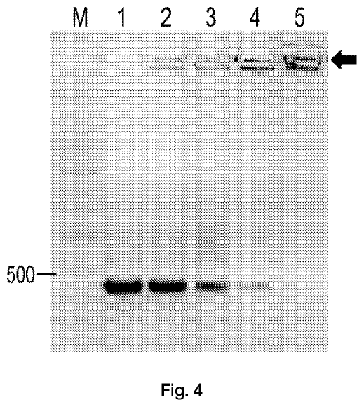

The efficacy of dsRNA-LDH, or BioClay, as an effective RNAi-based biopesticide for control of whitefly eggs and nymphs on cotton was assessed in plant-based insectary trials. The conventional untreated non-Bt cotton variety Sicot 620 at the 2-3 leaf stage were used for insectary trials. The most promising five target genes identified from artificial diet screening bioassays and twenty-two new candidate genes were selected based on their expression levels in different life-stages and critical functions in egg hatching and nymph survival (Table 3 and 4). These selected genes were tested against eggs and nymphs via plants-based insectary trials. The loading ratio of 1:3 dsRNA-LDH was used in all insectary trials instead of completely loaded BioClay ratio 1:4 (FIG. 4) to provide a small amount of free dsRNA which could be immediately available for eggs or nymphs triggering RNAi response from the moment of foliar spray application.

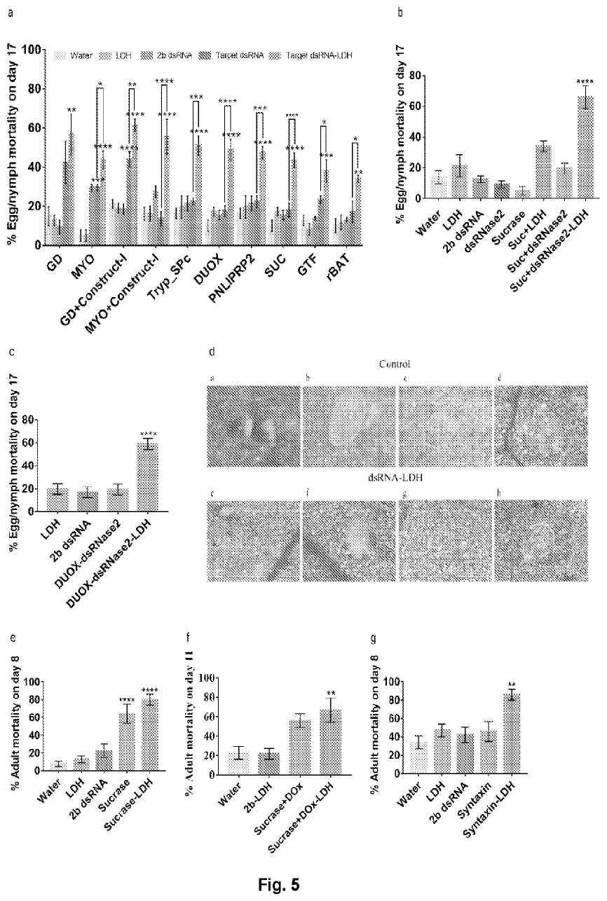

For plant-based egg and nymph trials, the adult whiteflies were released into clip cages on Cotton (Sicot 620) plants at two-three leaf stage and allowed oviposition for up to 18-24 hrs. Leaves with eggs present were sprayed on both sides with naked dsRNA and/or BioClay on day 1 and 10. The percentage mortality of eggs and nymphs were determined 17 days post first spray treatment. The sequence specificity of target dsRNA in inducing RNAi-mediated mortality of whitefly was confirmed using the CMV2b-dsRNA as a non-specific dsRNA control. The plants-based insectary trials were conducted by spraying the plants with water, LDH only, CMV2b-dsRNA, target dsRNA and target dsRNA-LDH (BioClay). Water, LDH and CMV2b-dsRNA treated plants showed an average of 5-29% eggs and nymph mortality on day 17 (FIG. 5a). In comparison, naked dsRNA targeting Glucose dehydrogenase (GD), Myosin (MYO), GD+Construct-I, MYO+Construct-I, Trypsin-like serine protease (Tryp_SPc), Dual oxidase (DUOX), Pancreatic lipase (PNLIPRP2), Sucrase (SUC), Glycosyl transferase (GTF) and rBAT showed only 17-44% eggs and nymph mortality, whereas target dsRNA-LDH resulted in 34-62% mortality compared to the water, LDH and CMV2b dsRNA treatments (FIG. 5a). Importantly, cotton plants sprayed with only dsRNA-LDH (BioClay) targeting MYO, GD+Construct-I, MYO+Construct-I, Tryp_SPc, DUOX, PNLIPRP2, SUC, GTF and rBAT resulted in significantly higher egg and nymph mortality compared to water and naked dsRNA treated plants (FIGS. 5a and 5d). However, plants sprayed with these naked dsRNA did not result in significant egg and nymph mortality than water treated plants (FIG. 5a). These results clearly demonstrate that foliar application of BioClay effectively controls whitefly eggs and nymphs than naked dsRNA.

A previous study reported that gut nucleases are major limitations to an efficient RNAi response in hemipteran insects, as they may cause degradation of orally ingested dsRNA1. To examine whether mixing of target dsRNA with whitefly nuclease-specific dsRNA (dsRNase2) can increase egg and nymph mortality, SUC (500 ng/μl) or DUOX dsRNA (500 ng/μl) mixed with dsRNase2 dsRNA (300 ng/μl) and LDH was added to form BioClay (1:3) and sprayed on cotton plants. Water, LDH and CMV2b-dsRNA controls sprayed cotton plants showed 13-21% egg and nymph mortality on day 17. dsRNase2, SUC and DUOX dsRNA only sprayed plants showed only 9%, 5%, and 19% mortality, respectively (FIGS. 5b and 5c). Importantly, plants sprayed with sucrase-dsRNase2-LDH and DUOX-dsRNase2-LDH resulted in 66% and 59% mortality, whereas sucrase-LDH showed only 34% and 19% mortality, respectively. These data demonstrate that mixing of target dsRNA with nuclease specific dsRNA can significantly improve the efficacy of RNAi in whitefly.

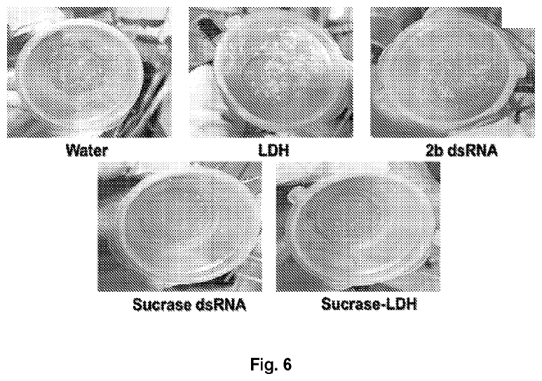

To further determine the effect of foliar spray of naked dsRNA and BioClay on survival of adult whiteflies was tested by plant-based insectary trials. Nineteen dsRNAs resulting in >50% mortality in artificial diet bioassays and newly identified seventeen dsRNAs targeting adult whiteflies were tested in plant-based insectary trials (Table 3 and 4). For insectary trials, Banjo penetrant (0.05%) was added to improve the uptake of dsRNA in plants. The cotton plants were sprayed with water, LDH, CMV2b-dsRNA, naked target dsRNA and LDH-target dsRNA (BioClay) on day 0. Approximately, 200 adult whiteflies per treatment group were released into 3-4 separate clip cages 24 hrs post-spray. Whiteflies fed on water, LDH, and CMV2b-dsRNA sprayed plants showed 8-42% mortality. Among the tested target dsRNAs, SUC (FIG. 5e), SUC+DUOX (FIG. 5f), and Syntaxin (FIG. 5g) caused 64%, 46%, and 56% mortality in naked dsRNAs and 80%, 85%, and 67% mortality in LDH-dsRNA (BioClay) treatments, respectively, after eight days. Unexpectedly, adult whiteflies fed on SUC dsRNA and SUC dsRNA-LDH showed reduction in honeydew secretion post 8 days (FIG. 6). These observations suggest a novel application of RNAi not only for controlling whiteflies but also for reducing secondary damage by the pest on cotton plants. RT-qPCR analysis demonstrated 30-89% knockdown in the expression of these target genes in adult whiteflies fed on dsRNA targeting each of these genes. These outcomes indicate that foliar application of dsRNA loaded on LDH or BioClay causes significant silencing of target genes and mortality in adult whiteflies through plants-based insectary trials.

Uptake and Internalization of dsRNA in Plants

For foliar application of dsRNA to provide RNAi-mediated control against hemipteran insects, the dsRNAs must travel through the leaf barriers such as waxy cuticle before reaching to the vascular bundle. One of the most crucial strategies to bypass the potential barriers to dsRNA uptake in plants via foliar application could be the incorporation of penetrant. Penetrants are adjuvants that can improve the absorption of active ingredients into plant tissues (Dubovik V, Dalinova A, Berestetskiy A. Effect of Adjuvants on Herbicidal Activity and Selectivity of Three Phytotoxins Produced by the Fungus, Stagonospora cirsii. Plants (Basel) 9(2020)). Penetrants such as Banjo (active ingredient—725 g/L Methyl ester (canola oil)) and Supercharge elite (active ingredient—471 g/L paraffin oil) increases the uptake of herbicide into the leaves by dissolving waxy cuticles, whereas Pulse penetrant improves the uptake through stomatal flooding.

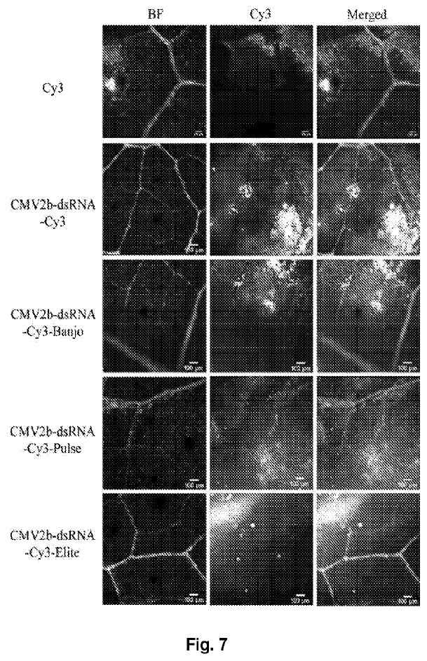

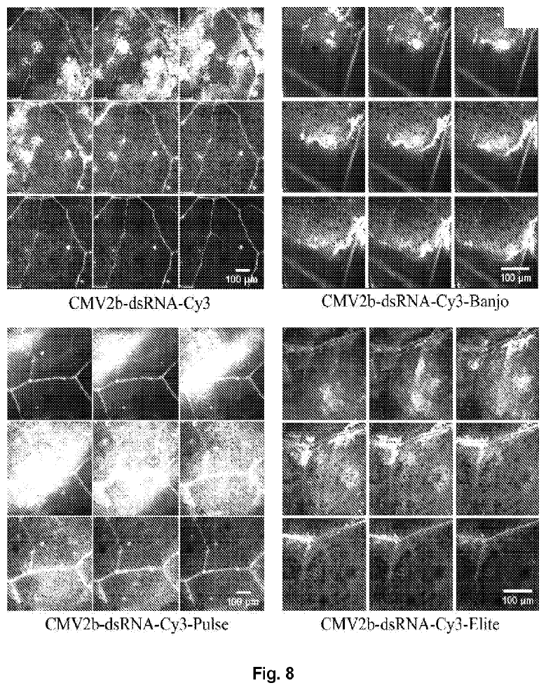

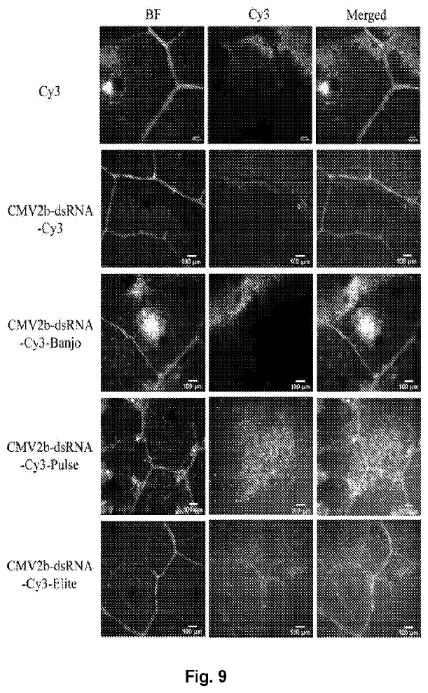

The uptake of Cy3-dsRNA into plant tissues was investigated by labelling the dsRNA with Cy3 fluorophores. Droplets of Cy3 only, fluorophore labelled CMV2b-dsRNA without penetrant or with either Banjo, Supercharge Elite or Pulse penetrants were topically applied to cotton leaves and visualized under confocal microscope 24 hours post-application for uptake of Cy3 into the leaf (FIG. 7a). Confocal imaging followed with Z-stack analysis of the adaxial (upper) surface of CMV2b-dsRNA-Cy3 treated leaves showed uptake of Cy3 in the spongy mesophyll, whereas no fluorescence was observed in leaves treated with Cy3-alone after rinsing (FIGS. 7a and 8. Importantly, all leaves treated with either of the penetrant showed abundant uptake and deeper penetration of Cy3 into the spongy mesophyll and plants vascular bundle (FIGS. 7, 8 and 9). Furthermore, translaminar movement of Cy3 fluorescence was observed in all leaves treated with CMV2b-dsRNA-Cy3-Penetrant, whereas leaves treated with CMV2b-dsRNA-Cy3 showed no movement of Cy3 into the leaf when visualized from the abaxial (lower) surface (FIG. 9). These results demonstrate that the addition of either of the penetrant to dsRNA can improve the uptake of dsRNA into the plants vascular bundle and translaminar movement of topically applied dsRNA into the plants.

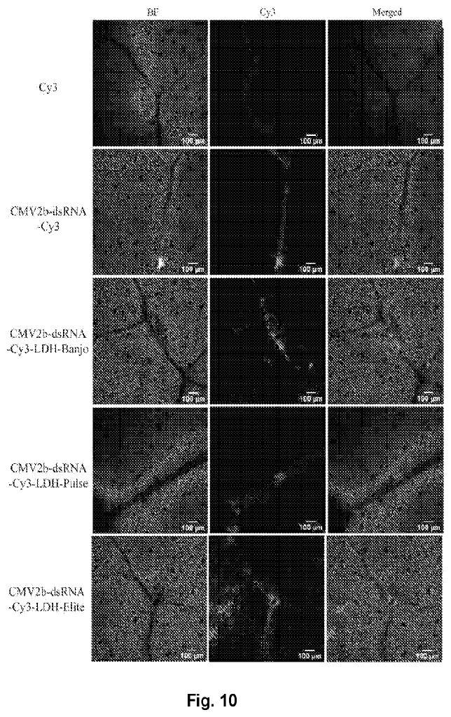

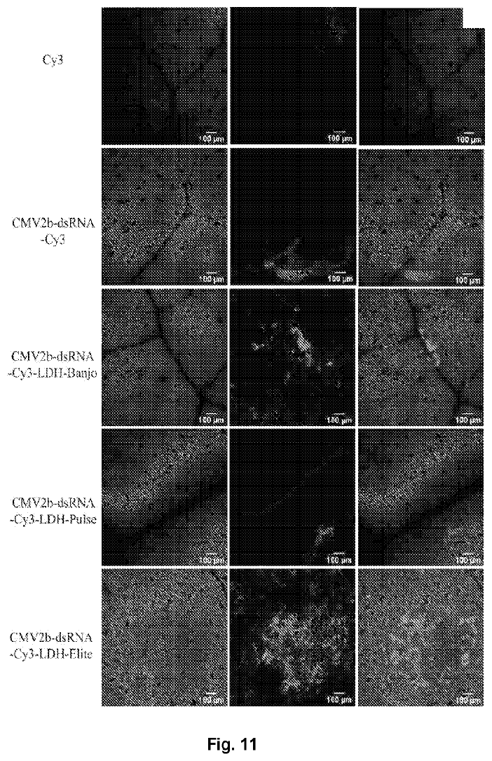

For dsRNA-LDH (BioClay) to provide RNAi-mediated control against whiteflies, the target dsRNA must not only be released on to the leaf surface, but also be taken up into the leaf. Naked CMV2b-dsRNA and completely bounded CMV2b-dsRNA-LDH (loading ratio 1:4) was used to assess the uptake of dsRNA into the leaf. Droplets of Cy3-Banjo penetrant, CMV2b-dsRNA-Cy3-Banjo, and CMV2b-dsRNA-Cy3-LDH-Banjo was topically applied to cotton leaves. After 72 hours of incubation, leaves were rinsed before visualisation under a confocal microscope. CMV2b-dsRNA-Cy3-Banjo and CMV2b-dsRNA-Cy3-LDH-Banjo treated leaves showed fluorescence in the spongy mesophyll and vascular bundle, whereas no fluorescence was observed in Cy3-Banjo treated leaves after rinsing (FIG. 10). Furthermore, Z-stack analysis was used to verify that the Cy3 fluorescence observed in the spongy mesophyll and vascular bundle was internalized (FIG. 11). These results suggest that both naked dsRNA and dsRNA released from the bound dsRNA-LDH complex were taken up by cotton plants.

TABLE 4
Number of target genes screened against developmental
stages of whitefly in artificial diet and plant assays

| Target whitefly life-stage | Number of genes | Screening assay |
|---|---|---|
| Adult | 63 | Artificial diet |
| Adult, Egg and Nymph | 5 | Artificial diet and Plant |
| Egg and Nymph | 22 | Plant |
| Adult | 17 | Plant |

Uptake of dsRNA in Whitefly

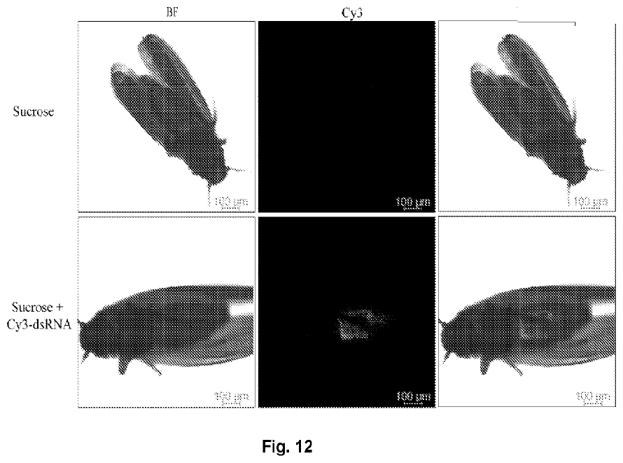

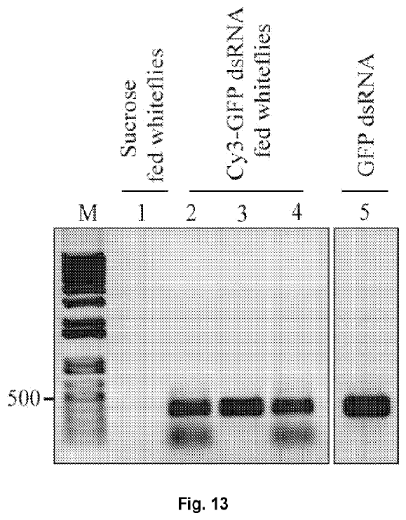

For efficient gene silencing, the whitefly must first take up exogenous dsRNA from the surrounding environment (artificial diet, detached leaf, or plant). The consumed dsRNA must then traverse the gut lumen into the insect gut cells. To assess whether dsRNA can be taken up by whiteflies through an artificial diet, sucrose only and sucrose-GFP-dsRNA-Cy3 were fed to adult insects. After 24 hours feeding, whole larvae were visualized under a confocal microscope for uptake of Cy3 into the insect. Clear Cy3-fluorescence was observed in the abdomen of the insects, whereas no fluorescence was visualized in the control sucrose fed larvae (FIG. 12). Combined with the fact that RNAi-mediated gene silencing is not limited to the site of dsRNA delivery, the spread of Cy3-fluorescence throughout the abdomen may suggest that systemic RNAi exist in whitefly. Furthermore, the uptake of GFP dsRNA in the whitefly was validated by RT-PCR followed by gel electrophoresis. Whiteflies fed on GFP-dsRNA-Cy3 showed uptake of GFP dsRNA, whereas no amplification was observed in the insects fed on only sucrose (FIG. 13). These observations clearly demonstrate that whitefly uptake dsRNA through artificial diet bioassays and these bioassays can be used for screening of potential target genes.

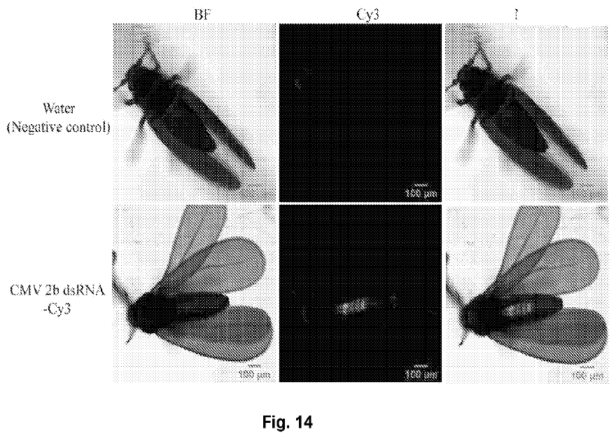

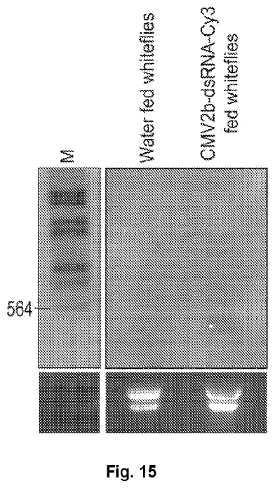

To assess the ability of whitefly to take up and internalize dsRNA via leaf feeding, detached cotton leaf petioles were dipped in a nuclease-free water (control) or CMV2b-dsRNA-Cy3 solution for 24 hours and adult whiteflies allowed to feed for a further 48 hours. Whole larvae examined under a confocal microscope showed Cy3-fluorescence signals in the gut when fed on CMV2b-dsRNA-Cy3 treated leaf tissue, whereas no fluorescence signal was observed in the whiteflies fed on water treated leaf tissue (FIG. 14). Additionally, the uptake of CMV2b-dsRNA in the whitefly was assessed by northern blot analysis. CMV2b-dsRNA was detectable in the whiteflies fed on CMV2b-dsRNA-Cy3 treated leaves, whereas no CMV2b-dsRNA was detected in whiteflies fed on water treated leaves (FIG. 15). These results clearly suggest that whiteflies can take up dsRNA through detached petiole dip assay and this method could be potentially useful in pest control platforms against hemipteran insects.

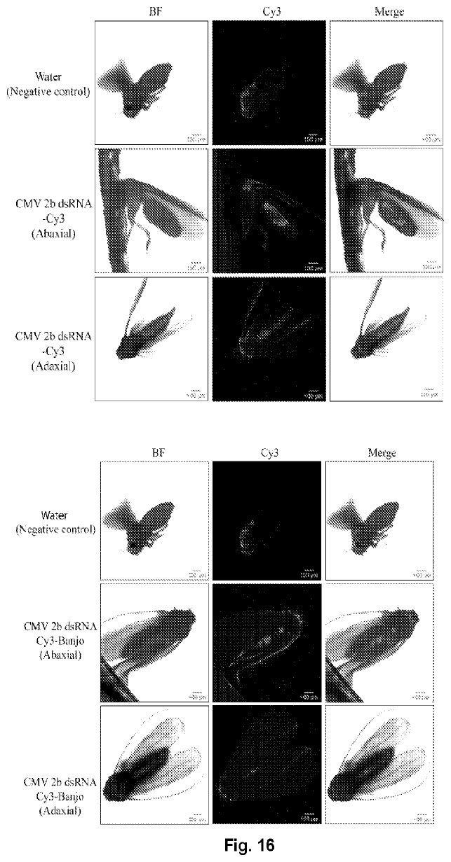

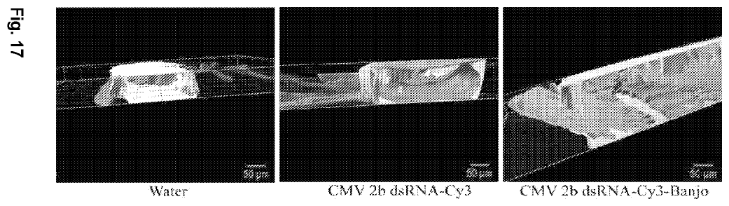

To further investigate the uptake of dsRNA into whiteflies through foliar application, droplets of nuclease free water (control), CMV2b-dsRNA-Cy3, and CMV-2b-dsRNA-Cy3-Banjo penetrant were applied onto the adaxial or abaxial surface of cotton leaves of fully intact plants. After 24 hours of incubation, adult whiteflies were released into clip-cages and allowed to feed on the abaxial side of the leaf for further 48 h before being visualized by confocal microscopy. Whiteflies fed on the leaf treated abaxially with CMV2b-dsRNA-Cy3 and CMV-2b-dsRNA-Cy3-Banjo penetrant showed a clear fluorescence signal in the gut of the insects, whereas no fluorescence signals were observed in whiteflies fed on water treated leaves (FIG. 16a). Interestingly, whiteflies fed on the leaf treated adaxially with dsRNA and dsRNA-penetrant showed a fluorescence signal in the gut, suggesting the insect can take up dsRNA from the phloem and not from the surface (FIGS. 16b and 17). These data clearly demonstrate that whitefly, a phloem feeder, can take up foliar applied dsRNA and that this delivery method could serve as a potential strategy for RNAi-mediated control of whiteflies.

Importantly this constitutes the first report of topically applied dsRNA uptake by whitefly feeding on intact plants. The current published literature is limited to artificial diet assays.

### DISCUSSION

The present inventors have identified ninety-four target genes in whitefly B. tabaci and extended their studies by three means: First, they used the diet feeding screening bioassays to identify potential RNAi target genes in the whitefly B. tabaci. dsRNAs targeting these novel RNAi target genes efficiently caused mortality in the whitefly and showed better performance than previously used genes. Secondly, the present inventors have tested whether RNAi efficiency could be increased by targeting two genes simultaneously in a single feeding bioassay. Both (i) mixing dsRNAs and (ii) stacking dsRNAs showed improved RNAi-mediated mortality in whitefly due to suppression of both the target genes. Third, the present inventors investigated the efficacy of LDH bounded dsRNA though diet feeding bioassays in whitefly. In this study, feeding of LDH bounded dsRNA showed significant gene knockdown and mortality in the whitefly B. tabaci. These results clearly demonstrate LDH nanosheets can effectively deliver dsRNA in the insect.

The present inventors have shown for the first time the foliar application of dsRNA using the LDH nanocarrier that can effectively control developmental stages of whiteflies, offering an alternative to conventional plant breeding and transgenic approaches. The use of RNAi-based foliar spray is shown to be effective against plant viruses (Mitter N, Worrall E A, Robinson K E, Li P, Jain R G, Taochy C, et al. Clay nanosheets for topical delivery of RNAi for sustained protection against plant viruses. Nature Plants 3:16207 (2017); Tenllado F, Diaz-Ruiz J R. Double-stranded RNA-mediated interference with plant virus infection. J Virol 75:12288-12297 (2001)), with little research against sap-sucking pests due to the limited transport of dsRNA into phloem cells considered a potential hurdle (Dalakouras A, Jarausch W, Buchholz G, Bassler A, Braun M, Manthey T, et al. Delivery of Hairpin RNAs and Small RNAs Into Woody and Herbaceous Plants by Trunk Injection and Petiole Absorption. Front Plant Sci 9:1253 (2018)). Previous studies have shown the successful application of nanoparticles to enhance stability and uptake of the target dsRNA in vivo (Zhang X, Zhang J, Zhu K Y. Chitosan/double-stranded RNA nanoparticle-mediated RNA interference to silence chitin synthase genes through larval feeding in the African malaria mosquito (Anopheles gambiae). Insect Mol Biol 19:683-693 (2010)). Here, the present inventors have shown that foliar application of BioClay can deliver the target dsRNA into vascular bundles and subsequently be taken up by whiteflies resulting in increased mortality of insects. Further, mixing of LDH bounded dsRNAs targeting sucrase and dsRNase showed increased mortality of whitefly eggs and nymphs via foliar spray. These findings demonstrate that in sap-sucking insects such as B. tabaci efficacy and success of RNAi against specific target genes can be improved by combination with dsRNA targeting the dsRNase genes.

Previous studies have shown the incorporation of transfection agents or the co-formulants, high pressure spraying, abrasion, and abaxial stomatal flooding can improve cuticle penetration and cellular uptake of siRNA (Dalakouras A, Wassenegger M, McMillan J N, Cardoza V, Maegele I, Dadami E, et al. Induction of Silencing in Plants by High-Pressure Spraying of In vitro-Synthesized Small RNAs. Front Plant Sci 7:1327 (2016); Huang, S., Iandolino, A., and Peel, G. (2018). Methods and Compositions for Introducing Nucleic Acids into Plants. U.S. Patent No. 20180163219A1). The present inventors have shown that addition of penetrant to the dsRNA can efficiently improve the uptake of dsRNA into the plant vascular bundles when topically applied onto the leaves (FIG. 7). The confocal microscopy followed with Z-stack image analysis showed that penetrant can assist the dsRNA to enter and transport to vascular bundles faster compared to the dsRNA without penetrant (Fig. S8). However, differentiating the dsRNA from the Cy3 fluorophore is challenging due to the overlapping wavelengths of Cy3 dye and chlorophyll that does not clearly confirm dsRNA uptake in plants. To address this issue, fluorescence lifetime imaging microscopy (FLIM) confocal platform was used that distinguish the autofluorescence from the leaf from the Cy3 fluorescence signal. The confocal microscopy coupled with Nanopore sequencing indicates that foliar applied dsRNA can be taken up by whitely. These observations suggest the possibility of transport of dsRNA through the phloem. Further, 3D surface rendering of the reflective and fluorescence mode was used to confirm the Cy3 fluorescence is inside the whitefly body and not on the outside surface (FIG. 17).

To summarize, the present inventors have clearly showed that the foliar application of BioClay can effectively control developmental stages of whiteflies. The study demonstrated the potential for BioClay platform using mixing of dsRNAs for improving RNAi effects.

1-45. (canceled)

46. A silencing element comprising at least one double-stranded RNA region, at least one strand of which comprises a polynucleotide that is complementary to:
(a) a nucleotide sequence comprising the sequence of any one of SEQ ID NOS: 139-173; and complements thereof;
(b) a nucleotide sequence comprising at least 70% sequence identity to the sequence of any one of nucleotides SEQ ID NOS: 139-173; and complements thereof; or
(c) a nucleotide sequence comprising at least 19 consecutive nucleotides of the sequence of any one of SEQ ID NOS: 139-173; and complements thereof;
wherein the silencing element has insecticidal activity against an insect plant pest.

47. The silencing element of claim 46, wherein the least one strand of which comprises a polynucleotide that is complementary to a nucleotide sequence comprising the sequence of any one of SEQ ID NOS: 139-173; or complements thereof.

48. The silencing element of claim 46, wherein the least one strand of which comprises a polynucleotide that is complementary to a nucleotide sequence comprising at least 70% sequence identity to the sequence of any one of nucleotides SEQ ID NOS: 139-173; or complements thereof.

49. The silencing element of claim 46, wherein the least one strand of which comprises a polynucleotide that is complementary to a nucleotide sequence comprising at least 19 consecutive nucleotides of the sequence of any one of SEQ ID NOS: 139-173; or complements thereof.

50. The silencing element of claim 46, wherein the least one strand of which comprises a polynucleotide that is complementary to a nucleotide sequence comprising at least 70% sequence identity to the sequence of any one of nucleotides SEQ ID NOS: 139-173; or complements thereof, with the nucleotide sequence comprising at least 19 consecutive nucleotides of the sequence of any one of SEQ ID NOS: 139-173; or complements thereof.

51. A composition comprising a silencing element comprising at least one double-stranded RNA region, at least one strand of which comprises a polynucleotide that is complementary to:
(a) a nucleotide sequence comprising the sequence of any one of SEQ ID NOS: 139-173; or complements thereof;
(b) a nucleotide sequence comprising at least 70% sequence identity to the sequence of any one of nucleotides SEQ ID NOS: 139-173; or complements thereof; or
(c) a nucleotide sequence comprising at least 19 consecutive nucleotides of the sequence of any one of SEQ ID NOS: 139-173; or complements thereof; and
wherein:
the silencing element is adsorbed onto LDH particles; and
the silencing element has insecticidal activity against an insect plant pest.

52. The composition of claim 51, wherein the least one strand of which comprises a polynucleotide that is complementary to a nucleotide sequence comprising the sequence of any one of SEQ ID NOS: 139-173; or complements thereof.

53. The composition of claim 51, wherein the least one strand of which comprises a polynucleotide that is complementary to a nucleotide sequence comprising at least 70% sequence identity to the sequence of any one of nucleotides SEQ ID NOS: 139-173; or complements thereof.

54. The composition of claim 51, wherein the least one strand of which comprises a polynucleotide that is complementary to a nucleotide sequence comprising at least 19 consecutive nucleotides of the sequence of any one of SEQ ID NOS: 139-173; or complements thereof.

55. The composition of claim 51, wherein the least one strand of which comprises a polynucleotide that is complementary to a nucleotide sequence comprising at least 70% sequence identity to the sequence of any one of nucleotides SEQ ID NOS: 139-173; and complements thereof, with the nucleotide sequence comprising at least 19 consecutive nucleotides of the sequence of any one of SEQ ID NOS: 139-173; and complements thereof.

56. The composition of claim 51, wherein the silencing element is loaded onto the LDH at a ratio of from 1:1 to 1:5 silencing element:LDH.

57. The composition of claim 51, wherein 60% to 90% of the silencing element in the composition is adsorbed onto the LDH.

58. A plant cell having stably incorporated into its genome a heterologous polynucleotide encoding a silencing element, wherein the polynucleotide comprises:
(d) a nucleotide sequence comprising the sequence of any one of SEQ ID NOS: 139-173; or complements thereof;
(e) a nucleotide sequence comprising at least 70% sequence identity to the sequence of any one of SEQ ID NOS: 139-173; or complements thereof; or
(f) a nucleotide sequence comprising at least 19 consecutive nucleotides of the sequence of any one of SEQ ID NOS: 139-173; or complements thereof;
wherein the silencing element has insecticidal activity against a plant pest.
